SEQUENCE LISTING (56 sequences)

SEQ ID NO: 1 - protein, 5 aa, Homo sapiens
KKEEE

SEQ ID NO: 2 - protein, 6 aa, Homo sapiens
KKEEEK

SEQ ID NO: 3 - protein, 10 aa, Homo sapiens
KKEEEKKEEE

SEQ ID NO: 4 - protein, 11 aa, Homo sapiens
KKEEEKKEEEK

SEQ ID NO: 5 - protein, 15 aa, Homo sapiens
KKEEEKKEEEKKEEE

SEQ ID NO: 6 - protein, 16 aa, Homo sapiens
KKEEEKKEEEKKEEEK

SEQ ID NO: 7 - protein, 5 aa, Homo sapiens
EEKKK

SEQ ID NO: 8 - protein, 6 aa, Homo sapiens
EEKKKE

SEQ ID NO: 9 - protein, 10 aa, Homo sapiens
EEKKKEEKKK

SEQ ID NO: 10 - protein, 11 aa, Homo sapiens
EEKKKEEKKKE

SEQ ID NO: 11 - protein, 13 aa, Homo sapiens
EEKKKEEKKKEEE

SEQ ID NO: 12 - protein, 14 aa, Homo sapiens
EEKKKEEKKKEEEK

SEQ ID NO: 13 - protein, 5 aa, Homo sapiens
EEEEE

SEQ ID NO: 14 - protein, 5 aa, Homo sapiens
KKKKK

SEQ ID NO: 15 - protein, 9 aa, Homo sapiens
MGSHIEPGG

SEQ ID NO: 16 - protein, 9 aa, Homo sapiens
KMGGTNHPE

SEQ ID NO: 17 - protein, 6 aa, Homo sapiens
GRGDSP

SEQ ID NO: 18 - protein, 9 aa, Homo sapiens
ELRGDRAKL

SEQ ID NO: 19 - protein, 18 aa, Homo sapiens
CKDSPKSSKSIRFIPVST

SEQ ID NO: 20 - protein, 6 aa, Homo sapiens
CKKEEE

SEQ ID NO: 21 - protein, 7 aa, Homo sapiens
CKKEEEK

SEQ ID NO: 22 - protein, 11 aa, Homo sapiens
CKKEEEKKEEE

SEQ ID NO: 23 - protein, 12 aa, Homo sapiens
CKKEEEKKEEEK

SEQ ID NO: 24 - protein, 16 aa, Homo sapiens
CKKEEEKKEEEKKEEE

SEQ ID NO: 25 - protein, 17 aa, Homo sapiens
CKKEEEKKEEEKKEEEK

SEQ ID NO: 26 - protein, 6 aa, Homo sapiens
CEEKKK

SEQ ID NO: 27 - protein, 7 aa, Homo sapiens
CEEKKKE

SEQ ID NO: 28 - protein, 11 aa, Homo sapiens
CEEKKKEEKKK

SEQ ID NO: 29 - protein, 12 aa, Homo sapiens
CEEKKKEEKKKE

SEQ ID NO: 30 - protein, 14 aa, Homo sapiens
CEEKKKEEKKKEEE

SEQ ID NO: 31 - protein, 15 aa, Homo sapiens
CEEKKKEEKKKEEEK

SEQ ID NO: 32 - protein, 6 aa, Homo sapiens
CEEEEE

SEQ ID NO: 33 - protein, 6 aa, Homo sapiens
CKKKKK

SEQ ID NO: 34 - protein, 10 aa, Homo sapiens
CMGSHIEPGG

SEQ ID NO: 35 - protein, 10 aa, Homo sapiens
CKMGGTNHPE

SEQ ID NO: 36 - protein, 7 aa, Homo sapiens
CGRGDSP

SEQ ID NO: 37 - protein, 10 aa, Homo sapiens
CELRGDRAKL

SEQ ID NO: 38 - protein, 6 aa, Homo sapiens
KKEEEC

SEQ ID NO: 39 - protein, 7 aa, Homo sapiens
KKEEEKC

SEQ ID NO: 40 - protein, 11 aa, Homo sapiens
KKEEEKKEEEC

SEQ ID NO: 41 - protein, 12 aa, Homo sapiens
KKEEEKKEEEKC

SEQ ID NO: 42 - protein, 16 aa, Homo sapiens
KKEEEKKEEEKKEEEC

SEQ ID NO: 43 - protein, 17 aa, Homo sapiens
KKEEEKKEEEKKEEEKC

SEQ ID NO: 44 - protein, 6 aa, Homo sapiens
EEKKKC

SEQ ID NO: 45 - protein, 7 aa, Homo sapiens
EEKKKEC

SEQ ID NO: 46 - protein, 11 aa, Homo sapiens
EEKKKEEKKKC

SEQ ID NO: 47 - protein, 12 aa, Homo sapiens
EEKKKEEKKKEC

SEQ ID NO: 48 - protein, 14 aa, Homo sapiens
EEKKKEEKKKEEEC

SEQ ID NO: 49 - protein, 15 aa, Homo sapiens
EEKKKEEKKKEEEKC

SEQ ID NO: 50 - protein, 6 aa, Homo sapiens
EEEEEC

SEQ ID NO: 51 - protein, 6 aa, Homo sapiens
KKKKKC

SEQ ID NO: 52 - protein, 10 aa, Homo sapiens
MGSHIEPGGC

SEQ ID NO: 53 - protein, 10 aa, Homo sapiens
KMGGTNHPEC

SEQ ID NO: 54 - protein, 7 aa, Homo sapiens
GRGDSPC

SEQ ID NO: 55 - protein, 19 aa, Homo sapiens
CKDSPKSSKSIRFIPVSTC

SEQ ID NO: 56 - protein, 10 aa, Homo sapiens
ELRGDRAKLC

(19) United States
(12) Patent Application Publication
(10) Pub. No.: US 2024/0197643 A1
(43) Pub. Date: Jun. 20, 2024

(54) ORAL DELIVERY OF NANOPARTICLES FOR KIDNEY DISEASE

(71) Applicant: UNIVERSITY OF SOUTHERN CALIFORNIA, Los Angeles, CA (US)

(72) Inventors: Eun Ji Paige CHUNG YOO, Los Angeles, CA (US); Jonathan WANG, Los Angeles, CA (US); Yi HUANG, Los Angeles, CA (US); Alysia COX, Los Angeles, CA (US)

(73) Assignee: UNIVERSITY OF SOUTHERN CALIFORNIA, Los Angeles, CA (US)

(21) Appl. No.: 17/998,618

(22) PCT Filed: May 11, 2021
(86) PCT No.: PCT/US2021/031714

§ 371 Date: Nov. 11, 2022

Related U.S. Application Data
(60) Provisional application No. 63/023,078, filed on May 11, 2020.

(51) Int. Cl. A61K 9/51; A61K 9/107; A61K 45/06; A61K 47/66; C12N 15/113

(52) U.S. Cl. CPC A61K 9/5161; A61K 9/1075; A61K 45/06; A61K 47/66; C12N 15/113; C12N 2310/141

(57) ABSTRACT

A drug delivery system for oral administration is provided. The drug delivery system includes an oral delivery carrier and a plurality of micelles attached to or dispersed in the oral delivery carrier. Each of the micelles includes a hydrophobic core and a hydrophilic corona targeted to a subject's kidney. A pharmaceutical payload is carried by the plurality of micelles.

Another drug delivery system includes a plurality of micelles and a payload within each micelle. Each micelle has a kidney targeting peptide conjugated thereto with a polyethylene glycol linking group having a molecular weight less than 1800 Daltons.

### CROSS-REFERENCE TO RELATED APPLICATIONS

This application is the U.S. national phase of PCT Appln. No. PCT/US2021/031714 filed May 11, 2021, which claims the benefit of U.S. provisional application Ser. No. 63/023,078 filed May 11, 2020, the disclosures of which are hereby incorporated in their entirety by reference herein.

### STATEMENT REGARDING FEDERALLY SPONSORED RESEARCH OR DEVELOPMENT

This invention was made with government support under DK121328-awarded by the National Institutes of Health and ROOHL124279 awarded by the National Heart, Lung, and Blood Institute. The government has certain rights in the invention.

### SEQUENCE LISTING

The text file USC0232PCT1_sequences_USC0232_ST25.TXT of size 12,288 created Sep. 27, 2023, filed herewith, is hereby incorporated by reference.

### TECHNICAL FIELD

In at least one aspect, the present invention is related to drug delivery systems composed of nanoparticles that include a plurality of micelles.

### BACKGROUND

Since the Food and Drug Administration (FDA) approved liposomal doxorubicin (Doxil) for cancer treatment in 1995, now more than 50 nano-pharmaceuticals are available for the clinic [1, 2]. Nanoparticles have been shown to improve drug therapeutic efficacy, reduce toxicity, and increase tissue selectivity compared to small molecule drugs [3]. Moreover, nanoparticles can combine multiple functionalities, including therapeutic and diagnostic capabilities, onto a single nanoparticle platform [4, 5] and have the potential to provide feedback on treatment effectiveness in real-time [6]. As such, nanomedicine has proven beneficial in the treatment of cancer [7, 8], multiple sclerosis [9], and human immunodeficiency virus (HIV) [10].

Despite these advances, similar developments in nanotechnology to improve the standard of care for chronic diseases, including chronic kidney diseases, are limited. For instance, autosomal dominant polycystic kidney disease (ADPKD) affects up to 12.5 million individuals worldwide [11], but no nanoparticle-based therapeutic efforts have been developed for clinical application [12, 13]. ADPKD is a slowly progressing, irreversible genetic condition characterized by cyst formation and enlargement arising from aberrant tubular epithelial growth and fluid secretion occurring throughout the kidney nephron, ultimately destroying kidney function [14, 15]. Several potential therapies have demonstrated benefits in slowing cyst growth in mice or clinical trials [16, 17], such as metformin, an FDA-approved drug for type 2 diabetes [18, 19], and tolvaptan, the only drug that was approved by the FDA in 2018. However, ADPKD preclinical studies administered drugs such as metformin at a higher dose (300 mg/kg/day) than currently prescribed (maximum 37.5 mg/kg/day for diabetic patients). Subsequently, adverse side effects such as gastrointestinal (GI) discomfort manifest in 25% of patients, and 5% of patients develop complete intolerance for the drug [20-22]. In addition, a more serious side effect of metformin is lactic acidosis, which has been found to be fatal in certain patients [23]. Tolvaptan, which is specifically prescribed to ADPKD patients with a rapidly progressing cyst phenotype [24], can also be a difficult drug to tolerate due to its many off-target side effects such as nausea, polyuria, muscle cramps, and idiosyncratic liver toxicity [25, 26]. The tolvaptan clinical trial dropout rate was significant at 23%, and projections show that after 18 years of continuous tolvaptan treatment, only a modest benefit of 4.9 year delay is achieved until kidney failure [27]. Therefore, new drug delivery approaches that can decrease systemic toxicity without compromising therapeutic efficacy is imperative for chronic diseases such as ADPKD.

Previously, kidney-targeted nanoparticle drug carriers using intravenous (IV) administration have been developed to enhance drug accumulation in the kidney and reduce such systemic side effects [12, 13, 28]. However, IV administration is not practical nor feasible in many cases for chronic diseases that progress over a lifetime, such as ADPKD [29]. Instead, oral drug delivery is the most convenient route of self-administration and results in the greatest treatment adherence [30, 31], preferred by 70% of patients [32]. Particularly for chronic conditions, oral delivery is attractive as it avoids needle complications such as infection, phlebitis, and pain [33, 34]. Additionally, patients with high needle fear and chronic, life-threatening health conditions (e.g., diabetes and multiple sclerosis) have been found to make important treatment decisions based on their aversity to needles over medical expertise [35-37], and hence, achieving a tolerable, self-administrable route is an important aspect to clinical success [38, 39].

Although favorable for patient compliance, orally-delivered drugs or nanoparticles must overcome unique physiological barriers that have historically limited their therapeutic efficacy [40]. These challenges include the acidic pH and enzymes present in the stomach that can degrade pharmaceutically active drugs [41], as well as the intestinal epithelial barrier that acts as a selectively permeable barrier to drugs for systemic circulation [42]. Moreover, even upon reaching the blood after absorption in the intestines, the first pass effect can metabolize up to 70-90% of orally administered drugs, rendering it therapeutically inactive through biotransformation [42, 44, 45]. Additionally, for chronic diseases, the long-term safety and tolerance of drug delivery systems that are orally taken will be vital.

One such material that can be offered to design oral drug delivery systems is chitosan. Chitosan-based materials have been proposed for oral delivery, as they offer many favorable properties such as biocompatibility, mucoadhesion, and tunability for controlled drug delivery [46,47]. Chitosan is derived from naturally occurring chitin found in the shells and exoskeletons of many crustaceans and is the second most abundant polysaccharide [48-50]. The purification process of chitin also allows tuning of the resultant chitosan, such as molecular weight, pKa (6-7.5) and degree of deacetylation properties, which provides a biomaterial that can be tailored for a wide range of biomedical applications [51,52].

Currently, chitosan is used in commercial biomedical products like the AQUANOVA Super-Absorbent Dressing, and is currently under clinical investigation as dental fillers (NCT03237624) and wound dressings (NCT03719261). Chitosan is considered Generally Recognized As Safe (GRAS) and edible by the FDA, but has not been directly approved for any nanoparticle drug delivery usage. The bottleneck may lie in the poor correlation between specific formulations or modifications of chitosan and the predicted in vivo response [53]. Hence, systematic studies assessing chitosan properties such as nanoparticle size and degree of acetylation are needed to exploit the beneficial properties of chitosan for drug delivery applications in the clinic [34,54,55].

Accordingly, there is a need for improved methods for delivering pharmaceuticals and other medical-related materials.

### SUMMARY

In some aspects, the properties of chitosan are advantageously used to develop oral nanomaterials that have optimal size, stability, and mucoadhesion to navigate through the GI tract and achieve efficient systemic delivery compared to free drugs [56]. Specifically, to form chitosan nanoparticles, several methods have been studied, including polyelectrolyte complexation [57], covalent cross-linking [58], complex coacervation [59], and ionotropic gelation [60-62]. We selected ionotropic gelation as the mild and aqueous processing conditions, non-toxic reagents, and ease of production is suitable for eventual clinical scale-up [63-66]. Moreover, chitosan nanoparticles have been previously shown to successfully deliver therapeutics in vivo, such as insulin [67,68], cyclosporin A, an immunosuppressant [69], and enoxaparin, an anticoagulant [70], further supporting its clinical suitability.

Due to these benefits, chitosan nanoparticles (CS-NP) are presented as an oral delivery platform for ADPKD and other chronic conditions. We show the synthesis parameters and their effect on the nanoparticle size, polydispersity, loading efficiency, and degradation rate in pH ranges that are present in the GI tract. We show the ability of these nanoparticles to permeate an intestinal barrier model and deliver the candidate ADPKD drug metformin in vitro [71]. Finally, we show successful oral delivery of chitosan nanoparticles loaded with metformin (CS-NP met) in a preclinical model of ADPKD, and demonstrate enhanced therapeutic efficacy compared to the free drug upon oral delivery. These results present the case for oral drug delivery applications using CS-NP, not only in the context of ADPKD and chronic diseases as provided herein, but treatments administered IV lacking the ability to overcome enteric barriers.

In another aspect, the nanoparticle that is loaded in CS-NP are a plurality of micelles attached to or dispersed in the oral delivery carrier. Each micelle includes a hydrophobic core and a hydrophilic corona targeted to a subject's kidney. A pharmaceutical payload is carried by the plurality of micelles.

In another aspect, a drug delivery system for oral administration includes an oral delivery carrier and a plurality of micelles attached to or dispersed in the oral delivery carrier. Each micelle includes a hydrophobic core and a hydrophilic corona targeted to a subject's kidney. A pharmaceutical payload is carried by the plurality of micelles.

In still another aspect, a drug delivery system includes a plurality of micelles and a payload conjugated to or encapsulated by each nanoparticle. Characteristically, each nanoparticle having a kidney targeting peptide conjugated thereto with a polyethylene glycol linking group having a molecular weight less than 1800 Daltons.

In another aspect, a drug delivery system includes a plurality of micelles and a payload carried by each micelle. Each micelle has a kidney targeting peptide, a linking group such as polyethylene glycol having a molecular weight less than 1800 Daltons.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIG. 1. Schematic of the oral delivery system carrying cargo.

FIG. 2. Schematic of a micelle that can be a payload in the oral delivery system of FIG. 1.

FIGS. 3A, 3B, 3C, 3D, and 3E. Optimization of CS-NP synthesis parameters and mucin binding efficiency. (A) Binding efficiency of chitosan to mucin increases upon chitosan deacetylation (DDA). (B) Nanoparticle diameter and (C) polydispersity as a function of the starting chitosan and poly-glutamic acid crosslinker concentration. The lowest polydispersity for CS-NP is seen at 2 mg/ml chitosan and 1 mg/ml crosslinker concentrations. (D) Diameter of CS-NP with respect to mucin binding efficiency shows the highest binding at approximately 150 nm. (*p≤0.05, **p≤0.01, N≥3) (E) TEM micrographs confirm spherical morphology and a monodisperse population of CS-NP with 90% DDA chitosan at 2 mg/ml and poly-L-glutamic acid crosslinker at 1 mg/ml synthesis conditions.

FIGS. 4A and 4B. In vitro pH response of CS-NP. (A) In vitro release of metformin from CS-NP under various pH conditions present in the GI tract (pH=1.2, fasting stomach; 2.5, fed stomach; 6.5 intestines, 7.4 blood; 1.3, SGF; 6.8 SIF) (N≥4). (B) TEM images of CS-NP confirm degradation at pH 6.5 and 7.4 after 6 h.

FIGS. 5A, 5B, 5C, and 5D. In vitro transport mechanisms and therapeutic efficacy of CS-NP (A) TER measurements of a Caco-2 cell layer upon 100 μM CS-NP R, free R, or PBS treatment 3 days before and after treatment show paracellular transport through tight junctions. (B) CS-NP R permeation across a Caco-2 cell layer treated with 100 μM of CSNP R after 4 h, pretreated with either colchicine (transcytosis inhibitor), wortmannin (macropinocytosis inhibitor) or no inhibitor. (C) Phosphorylated AMPK to total AMPK obtained via ELISA; and (D) ENaC current measurements of mpkCCDc14 cell monolayers treated for up to 48 h with CS-NP met (300 μM), free met, CS-NP, and PBS show a significant decrease for the CS-NP met and free met groups, compared to CS-NP, confirming therapeutic activity (****p≤0.0001, ***p≤0.001, N≥4).

FIGS. 6A, 6B, and 6C. Semi-quantitative biodistribution of mice treated with 10 mg/kg rhodamine (R) in 200 μL of CS-NP R and free R 24 h after oral gavage. (A) Comparison of ex vivo imaging between rhodamine fluorescence levels showed higher accumulation in the intestines for CS-NP R vs. free R 24 h post-oral gavage. (B) Serum fluorescence of CS-NP R shows a greater absolute bioavailability and an extended-release profile for the CS-NP formulation for up to 7 days (76.2% for CS-NP R and 47.9% for free R; ***p≤0.001, N≥4). (C) Representative ex vivo images confirm the highest signal in the intestines in the CS-NP R condition 24 h after oral gavage.

FIGS. 7A, 7B, and 7C. Quantification of intestinal localization of CS-NP R and free R 24 h after oral gavage. (A) Ex vivo fluorescence images and (B) quantitative comparison show the majority of CS-NP R adhered to the jejunum, while free R treatment is localized to the duodenum and ileum. (***p<0.001, N≥4). (C) Alcian blue staining of mucus shows colocalization of the CS-NP R to the intestinal mucosa, demonstrating mucoadhesion.

FIGS. 8A, 8B, and 8C. In vivo therapeutic efficacy of CS-NP loaded with metformin. (A) A lower KW/BW ratio (10.3±1.1 vs. 13.1±1.0 (**p≤0.01, N≥4) and (B) cystic index (57.6±1.2 vs. 66.5±0.8; **p<0.01, N≥4) was seen in the CS-NP met group vs. free drug. (C) H&E staining of whole kidneys shows less severe cystic phenotype in the CS-NP met group. A Cre-recombinase negative control is a non-diseased kidney morphology.

FIG. 9. TABLE 1; Serum components, electrolytes, and kidney health markers for CS-NC met, free met, and CS-NC treated mice show no significant difference between groups. Measured values include sodium (Na), potassium (K), chloride (Cl), ionized calcium (iCa), total carbon dioxide (tCO2), glucose (Glu), blood urea nitrogen (BUN)/Urea, creatinine (Crea), hematocrit (Hct), hemoglobin (Hb), and anion gap (AnGap). No alterations or toxicity are found.

FIGS. 10A, 10B, 10C, 10D, 10E, and 10F. TEM images of micelles. (A) 50% PEG2000-(KKEEE)₃K, (B) 50% PEG2000-(EEKKK)₃E, (C) 50% PEG2000-KKKKK, (D) 50% PEG2000-EEEEE, (E) 50% PEG1000-KKEEEKKEEEKKEEEK, and (F) 100% PEG5000-KKEEE KKEEEKKEEEK show spherical morphology. Scale bar: 100 nm.

FIGS. 11A, 11B, and 11C. Kidney accumulation of micelles upon intravenous administration after 24 hours. Comparison of micelles containing (A) varying number of peptide repeats of KKEEE or EEKKK, (B) charge differences, and (C) (KKEEE)₃K with various PEG molecular weight. n=4. *p<0.05, **p<0.01, ****p<0.0001.

FIGS. 12A, 12B, 12C, 12D, 12E, 12F, 12G, 12H, 12I, 12J, 12K, 12L, 12M, 12N, and 12O. Ex vivo images of kidney fluorescence after (A) 45% PEG2000-(KKEEE)₃K, (B) 45% PEG2000-(KKEEE)₂K, (C) 45% PEG2000-(KKEEE)K, (D) 45% PEG2000-(EEKKK)₃E, (E) 45% PEG2000-(EEKKK)₂E, (F) 45% PEG2000-EEKKKE, (G) 45% PEG2000-KKKKK, (H) 45% PEG2000-EEEEE, (I) 90% PEG2000-(KKEEE)₃K, (J) 90% PEG2000-(EEKKK)₃E, (K) 45% PEG1000-(KKEEE)₃K, (L) 90% PEG5000-(KKEEE)₃K, (M) 90% PEG2000-NT, or (N) PBS administration at 24 hours, (O) Legend.

FIGS. 13A, 13B, 13C, and 13D. Megalin staining and colocalization with micelles in kidney section of mice administered (A) 90% PEG2000-NT, (B) 45% PEG2000-(KKEEE)₃K, or (C) 45% PEG2000-(EEKKK)₃E. (D) Quantification of colocalization between micelles and megalin. Scale bar: 100 μm. *p<0.05, **p<0.01.

FIG. 14. H&E staining of kidney sections 24 hr after (a) 45% PEG2000-(KKEEE)₃K, (b) 45% PEG2000-(EEKKK)₃E, (c) 45% PEG2000-KKKKK, (d) 45% PEG2000-EEEEE, (e) 90% PEG2000-NT and (f) PBS administration. Scale bar: 100 μm.

FIGS. 15A, 15B, and 15C. Characterization of KM micelles loaded within CS NP. (A) DLS measurements show that the unloaded CS-NP diameter of 150 nm is preserved when up to 1000 uM of micelles are loaded within. (B, C) TEM images show spherical CS-NP KM of approximately 150 nm, with micelles dispersed throughout. Image analysis indicates 21.9+/−9.7 micelles per CS-NP. Scale bar: 100 nm.

FIGS. 16A, 16B, and 16C. Favorable pH responsive release of KM from CS-NP. (A) In vitro release of FITC labeled KM from CS-NP conditions present in the GI tract (pH=1.3 simulated gastric fluid (SGF); 6.8 simulated intestinal fluid (SIF)) (N≥4). Release is minimal (<25%) at low pH, but increases at pH conditions in the intestines, which suggests CS-NP payload will be protected in the stomach and released in the intestines. (B/C) DLS of supernatant at the final timepoint confirms intact micelle present in solution.

FIGS. 17A and 17B. CS-NP metformin is able to bypass an in vitro Transwell intestinal cell model and deliver therapeutic metformin to cells in the basolateral chamber. (A) Transwell schematic of mpkCCDc14 cell monolayers, with a Caco2 cell monolayer seeded on the transwell insert. (B) Phosphorylated AMPK to total AMPK obtained via ELISA of mpkCCDc14 treated for up to 48 h with CS-NP met (300 μM), free met, KM met, CS NP Blank, and PBS show a significant increase for the CS-NP met, KM met, and free met groups, compared to CS-NP, confirming therapeutic activity (****p≤0.0001, ***p≤0.001,N≥4).

FIGS. 18A-1, 18A-2, 18A-3, 18A-4, 18A-5, 18B-1, 18B-2, 18B-3, 18B-4, 18B-5, 18C-1, 18C-2, 18C-3, 18C-4, and 18C-5. CS-NP disrupts tight junctions in Caco-2 cell monolayers, an intestinal barrier model. Caco-2 cell monolayers were incubated with CS-NP met, free met, KM met, CS NP Blank, and PBS for 3 hours. Barrier tight junction integrity was stained with ZO-1, and cell nuclei with DAPI. Barrier disruption is only observed in groups containing chitosan.

FIG. 19. Bar graph demonstrating a decrease in proliferation when an ADPKD drug (metformin) and an epigenetic modifier are combined and delivered by micelles in ADPKD cells.

FIG. 20. Bar graph demonstrating a decrease in proliferation when an ADPKD drug (tolvaptan) and an epigenetic modifier are combined and delivered by micelles in ADPKD cells.

FIG. 21. Bar graph demonstrating a decrease in viability when a combined ADPKD drugs (metformin and tolvaptan) and an epigenetic modifier are combined and delivered by micelles in ADPKD cells.

FIG. 22. Bar graph showing cyst area in PKD1 heterozygous cells is reduced when an ADPKD drug (metformin) and an epigenetic modifier are combined. Cells are seeded in 2% Matrigel were incubated with either free 50 μM 5 Aza, 50 μM RG 108, or 5 μM TSA (dissolved DMSO, and added to complete media at 1% final vol/vol), as well as 300 μM metformin, or both 300 μM metformin in micelle formulations for up to 10 days hours, on a 96-well plate.

FIG. 23. Bar graph showing cyst area in PKD1 heterozygous cells is reduced when an ADPKD drug (tolvaptan) and an epigenetic modifier is combined. Cells seeded in 2% Matrigel were incubated with either free 50 μM 5 Aza, 50 μM RG 108, or 5 μM TSA (dissolved DMSO, and added to complete media at 1% final vol/vol), as well 10 μM Tolvaptan, or 10 μM Tolvaptan in micelle formulations for up to 10 days hours, on a 96-well plate.

FIG. 24. Bar graph showing cyst area in PKD1 heterozygous cells is reduced when combined ADPKD drugs (metformin and tolvaptan) and an epigenetic modifier is combined.

FIG. 25. Hydrodynamic diameter of micelles containing mTOR inhibitors, everolimus or rapamycinm at different concentrations, n=3. The green line indicates the size of empty micelles. The size of drug-containing micelles was compared to that of empty micelles using Student's t test (two-tailed), **p<0.01, ***p>0.001.

FIG. 26. Transmission electron microscopy (TEM) of representative micelles encapsulated with 10 uM everolimus or 10 uM rapamycin. All micelle concentrations are at 100 uM.

FIGS. 27A and 27B. MALDI-TOF spectra indicating the molecular weight of cortical collecting duct (CCD)-targeting peptides. Spectra indicated that peptides were successfully synthesized.

FIGS. 28A and 28B. MALDI-TOF spectra indicating the successful conjugation of CCD-targeting peptides to DSPE-PEG(2000)-maleimide.

FIGS. 29A, 29B, 29C, and 29D. Characterization of CCD-targeting micelles. A. Hydrodynamic diameter of micelles as measured by DLS. B. Zeta potential of micelles. C, D. TEM images of micelles.

FIGS. 30A and 30B. A) MALDI characterization of GRGDSPC (expected m/z: 731 g/mol). B) MALDI characterization of DSPE-PEG(2000)-GRGDSP (expected m/z: 3531 g/mol).

FIG. 31. MALDI characterization of DSPE-PEG(2000)-pravastatin (expected m/z: 3200 g/mol).

FIG. 32. TEM image of DSPE-PEG(2000)-pravastatin micelles.

FIG. 33. TEM image of octreotide-loaded micelles.

FIG. 34. TEM image of bardoxolone methyl-loaded micelles.

FIG. 35. TEM image of salsalate-loaded micelles.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B”. In the case of “only A”, the term also covers the possibility that B is absent, i e. “only A, but not B”

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

For all compounds expressed as an empirical chemical formula with a plurality of letters and numeric subscripts (e.g., CH₂O), values of the subscripts can be plus or minus 50 percent of the values indicated rounded to or truncated to two significant figures. For example, if CH₂O is indicated, a compound of formula C(0.8-1.2)H(1.6-2.4)((0.8-1.2). In a refinement, values of the subscripts can be plus or minus 30 percent of the values indicated rounded to or truncated to two significant figures. In still another refinement, values of the subscripts can be plus or minus 20 percent of the values indicated rounded to or truncated to two significant figures.

Throughout this application, where publications are referenced, the disclosures of these publications in their entireties are hereby incorporated by reference into this application to more fully describe the state of the art to which this invention pertains.

### Abbreviations

“ADPKD” means autosomal dominant polycystic kidney disease.
“Aza” means 5-aza-2′deoxycytidine (i.e decitabine).
“CCD” means cortical collecting duct.
“CS-NP” means chitosan nanoparticle.
“CMC” means critical micelle concentration.
“DLS” means dynamic light scattering.
“DSPE” means 1,2-distearoyl-sn-glycero-3-phosphoethanolamine.
“GFB” means glomerular filtration barrier
“(KKEEE)₃K means KKEEEKKEEEKKEEEK.
“(KKEEE)₂K” means KKEEEKKEEEK.
“KM” means kidney targeting peptide amphiphile micelle.
“MALDI” means matrix-assisted laser desorption/ionization.
“met” means metformin.
“mTOR” means mechanistic target of rapamycin.
“NHS” means N-hydroxysuccinimide.
“NT” means non-targeting micelle.
“PCT” means proximal tubule cell.
“PBS” means phosphate-buffered saline.
“PEG” means polyethylene glycol.
“PGA” means polyglycolic acid.
“PLA” means polylactic acid.
“PLGA” means poly(lactic-co-glycolic) acid.
“PM” means porcine mucin.
“PKD1” means polycystin1.
“PKD2” means polycystin2.
“PKHD1” means fibrocystin.
“RPTC” means renal proximal tubule cells.
“SIF” means simulated intestinal fluid.
“SGF” means simulated gastric fluid.
“TEM” means transmission electron microscopy.
“TSA” means trichostatin A.

### Definitions

The term “pharmaceutically acceptable carrier” means any material which, when combined with the compositions set forth herein allows the composition to retain biological activity. In the context of the present invention, a pharmaceutically acceptable carrier can include water or saline. Examples of other standard pharmaceutical carriers include a phosphate buffered saline solution, water, emulsions, such as an oil/water or water/oil emulsion, and various types of wetting agents. The compositions also can include stabilizers and preservatives.

The term “therapeutically effective amount” is an amount sufficient to effect beneficial or desired results. An effective amount can be administered in one or more administrations, applications or dosages.

The term “subject” refers to a human or animal, including all mammals such as primates (particularly higher primates), sheep, dog, rodents (e.g., mouse or rat), guinea pig, goat, pig, cat, rabbit, and cow.

The term “conjugate” means a compound formed as a composite between two or more molecules. In a refinement, the therapeutic agent is covalently bonded to an amphiphile typically via a linking group (e.g., PEG). Similarly, a fluorescent probe, when present is covalently bonded to an amphiphile via a linking group. Finally, the non-targeted amphiphile can include an end cap group conjugated to a base amphiphile. In other refinements, the composite between two or more molecules is formed by electrostatic interactions. In still other refinement, composite between two or more molecules is formed by hydrophobically incorporate ligands, drugs, and other molecules.

The term “amphiphilic” means a chemical compound possessing both hydrophilic and lipophilic properties.

The term “base amphiphile” refers to an amphiphile that can be reacted with a linking group that may attach another molecule or moiety (e.g., a fluorescent probe, a therapeutic agent, an end group, and the like).

The term “targeting peptide-conjugated amphiphile” refers to an amphiphile that is conjugated to a targeting peptide. Sometimes “targeting peptide-conjugated amphiphile” is referred to as “targeting peptide amphiphile.”

The term “non-targeted amphiphile” refers to an amphiphile that is not conjugated to a targeting peptide.

The term “payload” refers a component, material, or compound that is transported and delivered to a targeted organ (e.g., the kidney).

The term “pharmaceutical payload” refers a component, material, or compound that is transported and delivered to a targeted organ (e.g., the kidney) for the purpose of treating a disease (e.g., kidney disease).

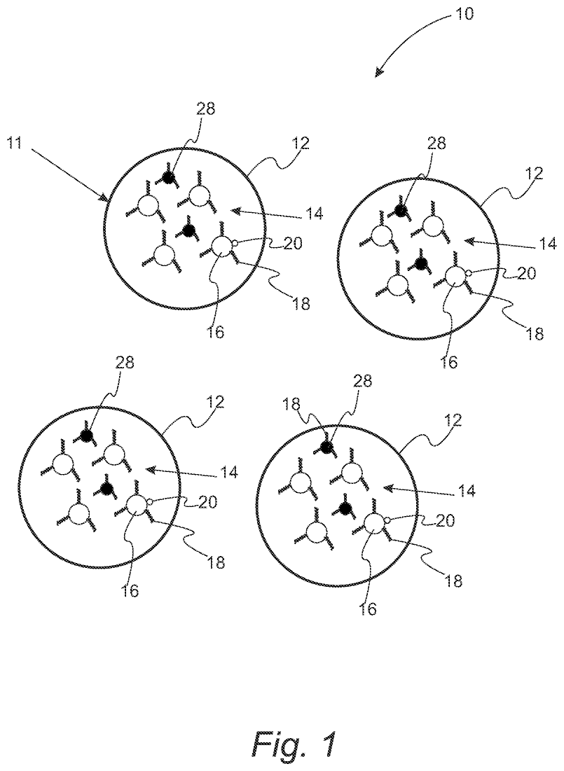

Referring to FIG. 1, a delivery system for oral administration is provided. The oral drug delivery system 10 includes a payload or one or more nanoparticles 11. In a refinement, the drug delivery system 10 includes a plurality of nanoparticles 11 (i.e., a first payload. Each nanoparticle 11 includes an oral delivery carrier 12 and a plurality of micelles 14 attached to or dispersed in the oral delivery carrier. Each micelle includes a hydrophobic core 16 and a hydrophilic corona 18. The hydrophilic corona 18 is typically targeted to a subject's kidney. A pharmaceutical payload 20 is carried by the plurality of micelles. In this regard, the pharmaceutical payload 20 can be located in a micelle surface and/or a micelle middle and/or a micelle core. Typically, the pharmaceutical payload is an agent that is useful for treating kidney disease.

In a refinement, the nanoparticles 11 have an average diameter less than about 300 nm. In a further refinement, the nanoparticles 11 have an average diameter from 50 nm to 200 nm.

As set forth below in more detail, the hydrophilic corona can include one or more kidney targeting peptides. Typically, the kidney targeting peptide is conjugated to the hydrophobic core with a linking compound. In one refinement, the kidney targeting peptide includes a sequence selected from the group consisting of KKEEE (SEQ ID NO: 1), KKEEEK (SEQ ID NO: 2), KKEEEKKEEE (SEQ ID NO: 3), KKEEEKKEEEK (SEQ ID NO: 4), KKEEEKKEEEKKEEE (SEQ ID NO: 5), and KKEEEKKEEEKKEEEK (SEQ ID NO: 6). In another refinement, the kidney targeting peptide includes a sequence selected from the group consisting of EEKKK (SEQ ID NO: 7), EEKKKE (SEQ ID NO: 8), EEKKKEEKKK (SEQ ID NO: 9), EEKKKEEKKKE (SEQ ID NO: 10), EEKKKEEKKKEEE (SEQ ID NO: 11), and EEKKKEEKKKEEEK (SEQ ID NO: 12). In still another refinement, the kidney targeting peptide includes a sequence selected from the group consisting of EEEEE (SEQ ID NO: 13), KKKKK (SEQ ID NO: 14), MGSHIEPGG (SEQ ID NO: 15), KMGGTNHPE (SEQ ID NO: 16), GRGDSP (SEQ ID NO: 17), ELRGDRAKL (SEQ ID NO: 18), and CKDSPKSSKSIRFIPVST (SEQ ID NO: 19).

In a variation, the kidney targeting peptide is selected from SEQ ID Nos: 1-19 with a cysteine added to the N-terminus or C-terminus thereof if a cysteine is not already present therein. The peptide sequences with a cysteine at the N-terminus are CKKEEE (SEQ ID NO: 20), CKKEEEK (SEQ ID NO: 21), CKKEEEKKEEE (SEQ ID NO: 22), CKKEEEKKEEEK (SEQ ID NO: 23), CKKEEEKKEEEKKEEE (SEQ ID NO: 24), CKKEEEKKEEEKKEEEK (SEQ ID NO: 25), CEEKKK (SEQ ID NO: 26), CEEKKKE (SEQ ID NO: 27), CEEKKKEEKKK (SEQ ID NO: 28), CEEKKKEEKKKE (SEQ ID NO: 29), CEEKKKEEKKKEEE (SEQ ID NO: 30), CEEKKKEEKKKEEEK (SEQ ID NO: 31), CEEEEE (SEQ ID NO: 32), CKKKKK (SEQ ID NO: 33), CMGSHIEPGG (SEQ ID NO: 34), CKMGGTNHPE (SEQ ID NO: 35), CGRGDSP (SEQ ID NO: 36), and CELRGDRAKL (SEQ ID NO: 37). The peptide sequences with a cysteine at the C-terminus are KKEEEC (SEQ ID NO: 38), KKEEEKC (SEQ ID NO: 39), KKEEEKKEEEC (SEQ ID NO: 40), KKEEEKKEEEKC (SEQ ID NO: 41), KKEEEKKEEEKKEEEC (SEQ ID NO: 42), KKEEEKKEEEKKEEEKC (SEQ ID NO: 43), EEKKKC (SEQ ID NO: 44), EEKKKEC (SEQ ID NO: 45), EEKKKEEKKKC (SEQ ID NO: 46), EEKKKEEKKKEC (SEQ ID NO: 47), EEKKKEEKKKEEEC (SEQ ID NO: 48), EEKKKEEKKKEEEKC (SEQ ID NO: 49), EEEEEC (SEQ ID NO: 50), KKKKKC (SEQ ID NO: 51), MGSHIEPGGC (SEQ ID NO: 52), KMGGTNHPEC (SEQ ID NO: 53), GRGDSPC (SEQ ID NO: 54), CKDSPKSSKSIRFIPVSTC (SEQ ID NO: 55), and ELRGDRAKLC (SEQ ID NO: 56).

In a variation, the polypeptides having sequences SEQ ID NOs: 1-56 include 1, 2, or 3 conservative substitutions. The conservative substitutions are similar to the amino acid be changed with respect to polarity, charge, solubility, hydrophobicity, hydrophilicity, and/or the amphipathic nature of the residues, while preserving the functionality of being constitutively active. Conservative substitutions that may be made are, for example, substitutions between aliphatic amino acids (alanine, valine, leucine, isoleucine), polar amino acids (glutamine, asparagine, serine, threonine), acidic amino acids (glutamic acid and aspartic acid), basic amino acids (arginine, lysine and histidine), aromatic amino acids (phenylalanine, tryptophan and tyrosine), large amino acids (phenylalanine and tryptophan), small amino acids (glycine, alanine) and hydroxyl amino acids (serine, threonine).

Although the present embodiment is not limited by the manner by which the kidney targeting peptide is conjugated to the micelle, the kidney targeting peptide can be connected to the micelles by reaction with a functional group attached to an end of a polyethylene glycol linking group. Examples of functional groups that can be used for linking include, but are not limited to amines, carboxylic acids, NHS esters, acid anhydrides, or unsaturated imides (e.g., maleimide).

In one variation, the linking compound that conjugates the kidney targeting peptide to the micelle is a polyethylene glycol having a weight average molecular weight from about 500 Daltons to 10000 Daltons. In a refinement, the linking compound is polyethylene glycol having a weight average molecular weight less than or equal to 1800 Daltons. Polyethylene glycols having a weight average molecular weight less than or equal to 1800 Daltons result in smaller micelles thereby facilitating the entry of the micelles into kidney tissues.

Typically, the pharmaceutical payload includes one or more pharmaceutical compound. In one refinement, the pharmaceutical payload is an mTOR inhibitor. In another refinement, the pharmaceutical payload is selected from the group consisting of pioglitazone, niacinamide, rapamycin, everolimus, tesevatinib, tolvaptan, metformin, somatostatin, octreotide, pasireotide, lixivaptan, venglustat, bardoxolone methyl, salsalate, curcumin, and combinations thereof. In another refinement, the pharmaceutical payload is selected from the group consisting of epigenetic modifying drugs including DNA methyltransferase inhibitors (e.g., Acytidine, Decitabine, RG108), and histone deacetylase inhibitors (e.g., Trichostatin A). In still another refinement, the pharmaceutical payload is a pravastatin, another statin, or combinations thereof. In yet another refinement, the pharmaceutical payload includes nucleic acids (e.g., microRNA-17 inhibitor), mRNA (e.g., encoding PKD1, PKD2, and/or PKHD1), aptamers, antibodies, and/or lectins.

An aspect of the drug delivery system is that it is designed for oral delivery of the pharmaceutical payload to a subject identified as having kidney disease. In this regard, the oral delivery carrier can include an enteric coating and/or materials such as gelatin. Examples of enteric coatings include, but are not limited to, cellulose acetate, hydroxypropyl methyl cellulose, methyl acrylate, or combinations thereof. In a variation, the oral delivery carrier includes chitosan, and in particular, a nano-sized chitosan nanoparticle capsule (i.e., a nano-sized chitosan particle) that encapsulates the pharmaceutical payload. In a refinement, the chitosan capsule includes crosslinked chitosan. Typically, the crosslinked chitosan includes chitosan that is crosslinked with polyglutamic acid or tripolyphosphate. The crosslinked chitosan can be unacetylated (e.g., a degree of acetylation less than about 10 mole percent acetylation) or acetylated (e.g., a degree of acetylation from about 70 to 98 mole percent). In a variation, the payload and/or the chitosan capsule includes a targeting peptide conjugated thereto. In a refinement, the targeting peptide sequence is selected from SEQ ID Nos: 1-19 or 1-56.

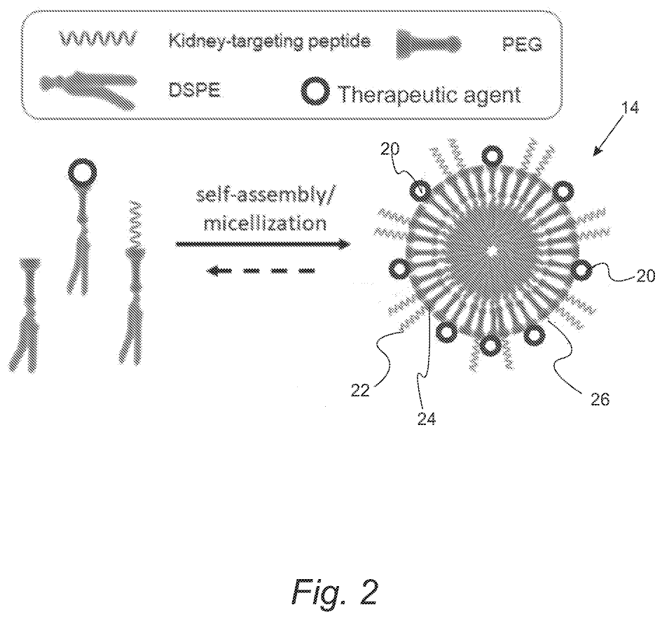

With reference to FIG. 2, a schematic of a micelle carrying a pharmaceutical payload is provided. Micelles 14 includes payload 20 (e.g., pharmaceutical compound) conjugated to or encapsulated by each micelle. Characteristically, each micelle has targeting ligands such as peptide(s) 22 conjugated thereto. Each micelle includes a plurality of targeting ligand conjugated amphiphiles 24 and a plurality of non-targeted amphiphiles 26. The plurality of targeting ligand-conjugated amphiphiles include amphiphiles having a polypeptide sequence selected from SEQ ID Nos: 1-19 or 1-56 conjugated to a base amphiphile. The non-targeted amphiphiles are characterized in not having a targeting peptide conjugated thereto. Typically, the molar ratio of the plurality of targeting peptide-conjugated amphiphiles to the plurality of non-targeted amphiphiles is from about 5:1 to 1:5 with a one-to-one ratio (1:1) being optimal. Typically, micelles 14 have an average diameter less than 50 nm. In a refinement, micelles 14 have an average diameter from about 5 to 30 nm.

In a refinement, the plurality of targeting peptide-conjugated amphiphiles includes a base amphiphiles that is conjugated to the targeting peptide with a linking group. Typically, the base amphiphiles include a phospholipid. Similarly, the non-targeted amphiphile can include a phospholipid that can be conjugated to an end group (e.g., C₁₋₁₀ alkoxyl) via a linking group. In a refinement, the linking group is a polyethylene glycol typically having a weight average molecular weight from about 500 Daltons to 10000 Daltons. The targeting peptide can be conjugated to the base amphiphile via reaction with a number of linking reactions known to those skilled in the art. Examples of functional groups that can be used for linking include amines, carboxylic acids, NHS esters, acid anhydrides, unsaturated imides (e.g., maleimide), and the like. In a refinement, the targeting peptide can be conjugated to the base amphiphile via reaction with a maleimide end group on the linking group as depicted in the following formula:

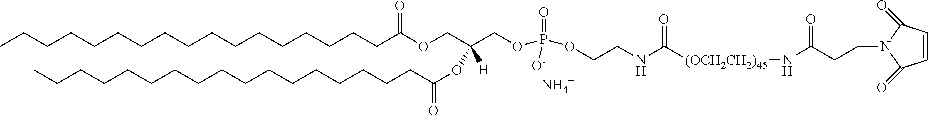

Therefore, the targeting peptide can be added by attaching a cysteine to the N-terminus of a targeting peptide selected from SEQ ID Nos: 1-19 or 1-56. This cysteine can add across the double bond in the maleimide group.

In a variation, phospholipids that can be used as the base amphiphile for the targeting peptide-conjugated amphiphiles and/or the non-targeted amphiphiles are selected from the group consisting of phosphatidic acids, phosphatidyl inositols, phosphatidyl cholines, phosphatidyl ethanolamines, phosphatidyl serines, phosphatidyl glycerols, and any combinations thereof. Specific examples of phospholipids that can be used include, but are not limited to, phosphatidylglycerol, lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine, palmitoyloleoylphosphatidylcholine, di stearoylphosphatidylcholine, dioleoylphosphatidylcholine, dipalmitoylphosphatidylcholine, dioleoylphosphatidylglycerol, dipalmitoylphosphatidylglycerol, -phosphatidylethanolamine, dioleoyl-phosphatidylethanolamine 4-(N-maleimidomethyl)-cyclohexane-1-carboxylate (DOPE-mal), 1-stearoyl-2-oleoyl phosphatidylcholine, 1,2-distearoyl-sn-glycerol-3-phosphoethanolamine, and combinations thereof. In a refinement, the phospholipid is selected from the group consisting of phosphatidic acids, phosphatidyl inositols, phosphatidyl cholines, phosphatidyl ethanolamines, phosphatidyl serines, phosphatidyl glycerols, and any combinations thereof.

Specific examples of targeting peptide-conjugated amphiphiles include amphiphiles selected from the group consisting of DSPE-PEG(2000)-CKKEEEKKEEEKKEEEK, DSPE-PEG(2000)-CKEEEKKEEEKKEEEKK, and combinations thereof. A specific example of a base amphiphile is described by the following formula:

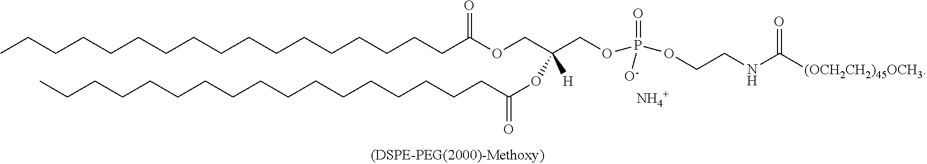

In another variation, the plurality of micelles includes micelles can include probe amphiphiles that include a fluorescent label. A specific example of a probe amphiphile having a Cy7 fluorophore is provided by the following formula:

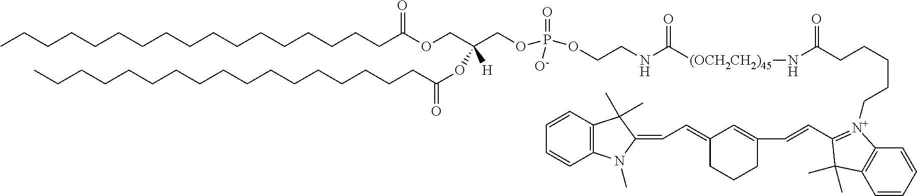

When the micelles of FIG. 1 are incorporated in the delivery system for oral administration of FIG. 1, the pharmaceutical payload conjugated to or encapsulated by the micelles is the same as set forth above.

In a variation, the micelles set forth above along with the payloads described in FIG. 2 can be administered without being attached to an oral delivery carrier. In this case, a drug delivery system includes a plurality of micelles carrying a payload by each micelle. Characteristically, each micelle having a kidney targeting peptide conjugated thereto with a polyethylene linking group having a weight average molecular weight less than 1800 Daltons. Payloads can be the pharmaceutical payloads set forth above as well as MRI agents (e.g., gadolinium, iron oxide), PET imaging radionuclides, and combinations thereof.

In another embodiment, a drug delivery system for oral administration is provided. Referring to FIG. 1, the drug delivery system 10 includes an oral delivery carrier 12 and a first payload 28 encapsulated and/or dispersed in the oral delivery carrier. Details of the oral delivery carrier are set forth above. In this embodiment, the first payload can be included in addition to or in place of the micelles described in FIG. 1. In a refinement, the first payload includes a plurality nanoparticles. Typically, the nanoparticles have average diameter from about 5 to 50 nm. In a variation, the first payload 28 can also include a pharmaceutical payload as set forth above. Moreover, the first payload and/or the oral delivery carrier can have the targeting peptides set forth above conjugated thereto. In a refinement, the first payload includes particles selected from the group consisting of micelles (as set forth above), liposomes metallic nanoparticles (i.e. gold nanoparticles), silica nanoparticles, polymeric nanoparticles (PLLA, PGA, PLGA), nucleic acid-type nanoparticles, MRI agents (e.g., gadolinium, iron oxide), PET imaging radionuclides, and combinations thereof.

In another embodiment, a method for treating a subject having kidney disease with the delivery system of FIG. 1 is provided. The method including a step of administering a therapeutically effective amount of the drug delivery system set forth above. In a refinement, the kidney disease is a chronic or acute kidney disease. Specific examples of kidney diseases that can be treated with the chitosan capsules include, but are not limited to, diabetic kidney disease, a tubulointerstitial disease, glomerulonephritis, Alport Syndrome, cystic kidney disease and/or polycystic kidney disease.

In another embodiment, a method for treating a subject having kidney disease with the delivery system including a plurality of micelles combined with an oral delivery carrier. The method including a step of administering a therapeutically effective amount of the drug delivery systems set forth above to a subject having kidney disease. In a refinement, the kidney disease is a chronic or acute kidney disease. Specific examples of kidney diseases that can be treated with the chitosan capsules include, but are not limited to diabetic kidney disease, a tubulointerstitial disease, glomerulonephritis, Alport Syndrome, cystic kidney disease and/or polycystic kidney disease. In this regard, the micelles can be administered intravenously, transdermally, interperitoneally, nasally, subcutaneously, buccally, sublingually, and inhaled.

Additional details regarding the micelles can be found in Wang J, Chin D, Poon C, Mancino V, Pham J, Li H, Ho P Y, Hallows K R, Chung E J. Oral delivery of metformin by chitosan nanoparticles for polycystic kidney disease. J Control Release. 2021 Jan. 10; 329:1198-1209. doi: 10.1016/j.jconrel.2020.10.047. Epub 2020 Oct 28. PMID: 33127449; PMCID: PMC7904655; and Huang Y, Kairui Jiang K, Zhang X, Chung E J, The effect of size, charge, and peptide ligand length on kidney targeting by small, organic nanoparticles. Bioeng Transl Med. 2020; 5:e10173. doi.org/10.1002/btm2.10173; the entire disclosures of which are hereby incorporated by reference in their entirety.

The following examples illustrate the various embodiments of the present invention. Those skilled in the art will recognize many variations that are within the spirit of the present invention and scope of the claims.

### 1. Oral Delivery of Metformin by Chitosan Nanoparticles for Polycystic Kidney Disease

### 1.1. Materials and Methods

### 1.1.1. Materials

Chitosan with 95% degree of deacetylation and average molecular weight 150 kDa was purchased from Heppe Medical (Germany). Mucin type II from porcine stomach, Rhodamine B, pharmaceutical grade met, and poly-L-glutamic acid were purchased from Sigma-Aldrich (USA). All other reagents were of analytical grade.

### 1.1.2. Synthesis of CS-NP

Chitosan (0.5, 1.0, 1.5, 2.0, 2.5, and 3.0 mg/ml) was dissolved in MilliQ water containing 0.5% glacial acetic acid, sonicated and vortexed to obtain homogenous mixtures. Similar concentrations (0.5, 1.0, 1.5, 2.0, 2.5, and 3.0 mg/ml) of poly-L-glutamic acid solutions were prepared in MilliQ water. Chitosan solution was added dropwise to polyglutamic acid under constant stirring in a round bottom flask at 600 rpm; an opalescent solution was seen upon successful formation of nanoscale particles. The final solution was centrifuged at 14,000 rpm for 30 minutes at 14° C. The resulting pellet was serially washed with 20%, 75%, and 100% ethanol. To encapsulate a payload, met or rhodamine B was dissolved at desired concentrations in poly-glutamic acid solution and used during nanoparticle synthesis. Loading efficiency of the payload was calculated by quantifying the amount of unincorporated met or rhodamine remaining in the supernatant. Rhodamine fluorescence was measured at λexcitation=553 nm and λₑₘᵢₛₛᵢₒₙ=630 nm, while met absorbance was measured at 233 nm using a Varioskan LUX plate reader [65, 66] (Thermo Fisher Scientific, Waltham, MA, USA).

### 1.1.3. Dynamic Light Scattering (DLS)

Nanocapsules derived from chitosan (0.5-3.0 mg/ml) and poly-L-glutamic acid (0.5-3.0 mg/ml) concentrations were dispersed in 63 μL of MilliQ water and measured by DLS to confirm size and polydispersity index (PDI). DLS measurements were determined at 163.5° and 532 nm using a Wyatt Technology Möbiuζ system (Santa Barbara, CA, USA, N≥3). All measurements were carried out at 25° C. after equilibrating for 5 minutes.

### 1.1.4 Zeta Potential

The zeta potential of chitosan nanocapsules was measured using the same Möbiuζ system described above. Samples were placed in a Quartz cuvette with a polyether ether ketone (PEEK) and platinum dip probe (N≥3) and measurements were carried out at 25° C.

### 1.1.5 Transmission Electron Microscopy (TEM)

TEM samples were prepared by placing 7.0 μL of chitosan nanocapsules in MilliQ water on 400 mesh lacey carbon grids (Ted Pella, Redding, CA, USA) for 5 minutes. Excess liquid was wicked away with filter paper and the grid was washed with MilliQ water before placing 2 wt. % uranyl acetate solution for 2 minutes. After washing once more with MilliQ water, samples were dried and immediately imaged on a JEOL JEM-2100F (JEOL, Ltd., Tokyo, Japan).

### 1.1.6 Re-Acetylation of Chitosan

Chitosan with ˜95% deacetylation was re-acetylated to achieve varying degrees of N-deacetylated chitosan. Chitosan (2 mg/ml) was mixed with 200 mM acetic anhydride in a 50:50 methanol/water mixture at 95° C. and stirred for 1-12 hours. Confirmation of deacetylation degree was measured from the first derivative of the UV-vis absorption spectra obtained from a Varioskan LUX plate reader as specified by de Silva et al. [67]. Reaction times between 1-6 hours produced chitosan with 95-85% degree of deacetylation, while 6-12 hours and >12 hours resulted in 70-85% and 55-70% deacetylation, respectively.

### 1.1.7 Mucin-Binding Assay

Porcine mucin (PM) in phosphate buffer (pH 7.4) was incubated with CS-NPs of varying degrees of deacetylation verified by UV-vis, (90, 80, 70, 50% deacetylation, 50-300 nm diameter) at room temperature (23° C.) for 2 h (1,1, v/v), before centrifugation for 60 min at 14,000 rpm and 14° C. Absorbance of the remaining free PM in the supernatant was measured by UV spectrophotometry at 251 nm. The mucoadhesiveness was expressed as PM binding efficiency calculated by the following equation:

$$binding efficiency = \frac{{c}_{0} - {c}_{s}}{{c}_{0}} \times 100 \qquad ( 1 )$$

where Cₒ is the initial concentration of PM used for incubation (400 μg/mL) and Cs is the measured concentration of free PM in the supernatant after removal of chitosan-bound PM. The standard curve was determined using 50, 100, 150, 200, 250, 300, 350 g/mL PM solutions.

### 1.1.8 Drug Release and Morphological Response to pH

Drug release studies were performed on nanoparticles (2 mg/ml initial chitosan and 1 mg/ml poly-L-glutamic acid) suspended in PBS adjusted to pH 1.2, 2.5, 6.5, or 7.4 with the addition of HCl or NaOH, simulated gastric fluid (SGF) composed of 2.0 g/L sodium chloride and 2.9 g/L HCl (pH 1.3), or simulated intestinal fluid (SIF) composed of 0.62 g/L sodium hydroxide and 6.8 g/L potassium phosphate monobasic (pH 6.8) [75]. Free met released from nanoparticles was quantified at 233 nm using a NanoDrop One microvolume UV-Vis spectrophotometer for up to 6 h at room temperature (Thermofisher Scientific, Waltham, MA, USA).

To assess the morphology of nanoparticles in response to pH conditions, particles were immersed in each pH condition for 6 h, and imaged via TEM. All experiments were carried out in triplicate.

### 1.1.9 Cell Culture

Human colon epithelial cells (Caco-2, ATCC HTB-37, ATCC, Manassas, VA, USA) were cultured following the manufacturer's recommendations. Cells were expanded in Dulbecco's Modified Eagle Medium (FBS) and 1% penicillin-streptomycin. Cells were seeded at a density of 4.5×10³ cells/cm2 and subcultured upon 50% confluence [76].

Mouse kidney cortical collecting duct (mpkCCDc14) cells were expanded in culture media comprised of DMEM/F12 (11054-054, Waltham, MA, USA) supplemented with insulin, dexamethasone, selenium, transferrin, triiodothyronine, glutamine, D-glucose, epidermal growth factor (EGF), HEPES, sodium pyruvate as outlined by Bens et al. [77]. Complete media was filtered before use, and media was changed every two days and subcultures were passaged every 7-8 days. Both cell lines were grown at 37° C. in a humidified incubator under 5% CO2.

### 1.1.10 In Vitro Cell Compatibility

Biocompatibility was assessed with an MTS cell proliferation colorimetric assay following the manufacturer's instructions (BioVision Incorporated, San Francisco, CA, USA). MpkCCDc14 (5000 cells/well) or Caco2 (5000 cells/well) were incubated with either 10, 50, 100, or 500 μM of CS-NP for 24 h on a 96-well plate before the addition of MTS reagent. Assay fluorescence was measured via a Varioskan LUX plate reader (Thermo Fisher Scientific, Waltham, MA, USA).

### 1.1.11 Transepithelial Resistance (TER) Surveillance

For transport and monolayer resistance experiments, Caco-2 cell monolayers were seeded onto Transwell inserts (Corning, NY, USA; diameter 6.5 mm, growth area 0.33 cm², pore size 0.4 μm), at an initial density of 3×10⁵ cell/cm2 and maintained for 21 days in complete medium to form a confluent monolayer. The change of TER, representing the tightness of the cell monolayers, was measured by an EVOM2 Epithelial Voltohmmeter (World Precision Instruments, USA). Monolayers reaching steady-state values in the range of 300-400 Ω-cm² were used for studies [76].

### 1.1.12. Cellular Uptake and Transport of CS-NP

To assess the cellular uptake pathways of chitosan nanoparticles, Caco-2 cells were pretreated with medium containing colchicine (transcytosis inhibitor, 10 μM, for 60 min) or wortmannin (macropinocytosis inhibitor, 0.06 mM, for 3 h) [78,79] before 100 μL of 100 μM rhodamine-loaded chitosan nanoparticles (CS-NP R) was administered in the apical chamber. The amount of rhodamine fluorescence was measured in the basolateral chamber over the course of 4 h.

Additionally, the effect of paracellular transport through tight junctions was monitored via TER of Caco-2 cells seeded on Transwell inserts. TER measurements were performed daily for three days prior to treatment with CS-NP R, free Rhodamine (free R), or PBS control to establish baseline measurements. TER was monitored every 6 h, then again on day 2 and 3 post-administration.

### 1.1.13 In Vitro Therapeutic Efficacy of CS-NP Met Through ELISA and Epithelial Sodium Channel (ENaC) Measurements

To assess therapeutic efficacy in vitro, the cellular levels of phosphor-AMPK (Kit #7959) and total AMPK (Kit #7961) were measured via enzyme-linked immunosorbent assays (ELISA, Cell Signaling Technologies, Danvers, MA, USA) according to the manufacturer's instructions. mpkCCDc14 cells were treated for 12 h with 300 μM of met in CS-NP met or free met, and were compared to CS-NP or PBS treated controls. All standards and samples were measured on a Varioskan LUX microplate reader at a wavelength of 450 nm.

To validate the therapeutic efficacy of met on the reduction of ENAC current, TER and potential difference (PD) measurements were made on mpkCCDc14 cells seeded on Transwell filters as described above [20]. ENaC-dependent equivalent short-circuit currents (leq) was estimated by Ohm's law, dividing the measured PD by the TER value and the area of one Transwell membrane (0.33 cm2). mpkCCDc14 cell cultures were treated with 100 μL CS-NP met (300 μM met), CS-NP, or PBS at pH 7.4.

### 1.1.14 Ex Vivo Imaging of Orally Administered CS-NP

To assess the biodistribution of CS-NP semi-quantitatively, 6-7 week old male and female C57BL/6 J mice (Jackson Laboratories, Bar Harbor, ME, USA) were orally gavaged with 10 mg/kg of rhodamine loaded in 200 μL of 500 μM CS-NP R, 10 mg/kg of free R, or PBS control. Mice were euthanized after 3, 24, or 48 h post-injection and organs (e.g., brain, heart, lungs, liver, kidneys, spleen, intestines, and bladder) were excised and imaged ex vivo on an AMI HTX in vivo imaging system (Spectral Instruments Imaging, Tucson, AZ, USA). The fluorescence signal was quantified via Aura software (Spectral Instruments Imaging, Tucson, AZ, USA, N ≥4), and the background was subtracted from the PBS treated group. The mean radiance (photons/s/cm2/sr) for each organ was quantified as a region of interest, and % of total organ fluorescence was obtained by dividing each organ by the sum of all the organ regions. Urine and blood samples were collected following organ harvest and stored at −20° C. until further analysis. All animal procedures followed NIH guidelines for the care and use of laboratory animals and were approved by the University of Southern California's Institutional Animal Care and Use Committee.

### 1.1.15 In Vivo Half-Life of CS-NP

To compare serum half-life, 200 μL of CS-NP R or free R was administrated at a dose of 10 mg/kg loaded rhodamine via oral gavage on 6-7-week-old male and female C57BL/6 J mice (N≥4). Blood draws were performed either retro-orbitally or via tail vein at 30 min, 3, 6, 12, 24, 36, and 48 h post-administration. Fluorescence was measured in serum and quantified using a rhodamine calibration curve developed in mouse serum. Absolute bioavailability was calculated for CS-NP R and free R relative to mice IV injected via tail vein with 10 mg/kg rhodamine dissolved in 100 μL of PBS.

### 1.1.16 Histology and Immunohistochemistry

Following ex vivo imaging, the brain, heart, lungs, liver, kidneys, spleen, intestines, and bladder were immediately frozen and embedded in OCT (Tissue Tek, Sakura Finetek, Torrance, CA, USA). 10 μm sections were obtained (CM3050 S Cryostat, Leica, Nussloch, Germany) and placed on Superfrost Plus slides (Fisherbrand, Waltham, MA, USA). Tissue sections were stained with hematoxylin & eosin (H&E) and imaged (Leica DMi8, Leica, Wetzlar, Germany, N=3). For staining of intestinal mucin, tissue sample were processed with an alcian blue 1%, pH 2.5 stain kit (Newcomer Supply, Middleton, WI, USA). Briefly, tissue sections on slides were washed in acetic acid for 3 min, incubated in alcian blue for 30 min at 4° C. in a humidified chamber, and counterstained with Nuclear Fast Red (Vector Laboratories, Burlingame, CA, USA). Samples were mounted using VectaMount™ (Vector Laboratories, Burlingame, CA, USA). 1.17 Kidney health in PKD mice Serum components, electrolytes, and kidney health markers including sodium (Na), potassium (K), chloride (Cl), ionized calcium (iCa), total carbon dioxide (tCO2), glucose (Glu), blood urea nitrogen (BUN)/Urea, creatinine (Crea), hematocrit (Hct), hemoglobin (Hb), and anion gap (AnGap) were assessed in diseased Pkd1fl/fl; Pax8 rtTA; Tet-O-Cre mice. 90 μL of blood taken from the submandibular vein on the day of harvest was analyzed using Chem-8+ cartridges for the i-Stat Handheld Blood Analyzer (Abbott, Chicago, IL, USA).

### 1.1.17. Therapeutic Efficacy in ADPKD Mice

To assess the ability CS-NP to enhance the therapeutic efficacy of orally administered drugs in ADPKD, 300 mg/kg met-loaded CS-NP or free met was administered to Pkd1fl/fl; Pax8 rtTA; Tet-O-Cre mice. Pups were IP injected with doxycycline on postnatal day 10-11 (P10-11) to induce a severe PKD phenotype as previously described [80]. Mice were orally gavaged every two days starting on P12 and euthanized on P22 (N≥4). Kidneys were excised to assess kidney to body weight (KW/BW) ratio and stained with H&E to compare cystic index. Cystic index was defined as the percentage of cystic area divided by total kidney area and determined by ImageJ.

### 1.1.18. Kidney Health in PKD Mice

Serum components, electrolytes, and kidney health markers including sodium (Na), potassium (K), chloride (Cl), ionized calcium (iCa), total carbon dioxide (tCO2), glucose (Glu), blood urea nitrogen (BUN)/Urea, creatinine (Crea), hematocrit (Hct), hemoglobin (Hb), and anion gap (AnGap) were assessed in diseased Pkd1fl/fl; Pax8 rtTA; Tet-OCre mice. 90 μL of blood taken from the submandibular vein on the day of harvest was analyzed using Chem-8+ cartridges for the i-Stat Handheld Blood Analyzer (Abbott, Chicago, IL, USA).

### 1.1.19. Statistical Analysis

A Student's t-test was used to compare the means of pairs. Analysis of variance (ANOVA) with Tukey's multiple comparison test post-hoc analysis was used to determine significant differences among three or more means. A p-value of ≤0.05 was considered to be significant.

### 1.2. Results and Discussion

### 1.2.1. Fabrication and Characterization of Chitosan Nanocapsules

Chitosan is an easily procured biomaterial, and several synthesis methods have been investigated to synthesize chitosan nanoparticles [82-85]. Ionic gelation was used in this study as it has been reported to achieve high drug encapsulation and low polydispersity [64,65], and ionic gelation is based on electrostatic interaction between the amine group of chitosan and a negatively-charged group of a polyanion such as poly-L-glutamic acid [61]. Due to this charge-based interaction, negatively-charged payload drugs can also be easily incorporated during the ionic gelation process [86].

In addition, one of the main advantages of using chitosan for oral delivery is its mucoadhesive properties, as the mucous lining in the GI tract provides a significant barrier to many drugs [87]. Mucous is composed of water (˜90 to 98%), salts (˜0.5 to 1.0% w/w), proteins (˜0.5% w/v), and mucins (0.2-5% w/w) which are the glycoproteins responsible for excluding large micrometer-sized particulates by steric hindrance [89]. Without mucoadhesive properties, oral delivery formulations lack the ability to withstand peristalsis movements as well as the extensive washing effect of body fluids, such as GI acids [91], which results in the loss of drug payload available systemically.

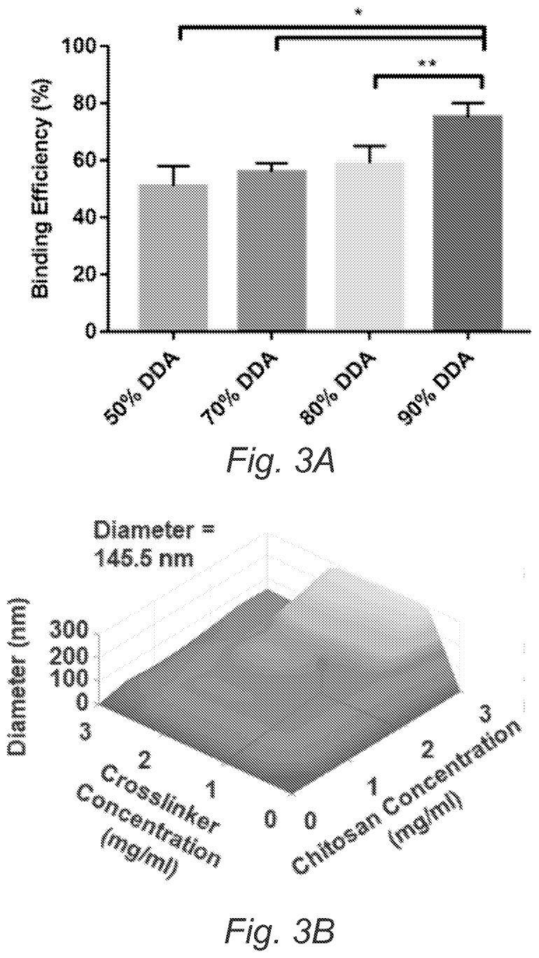

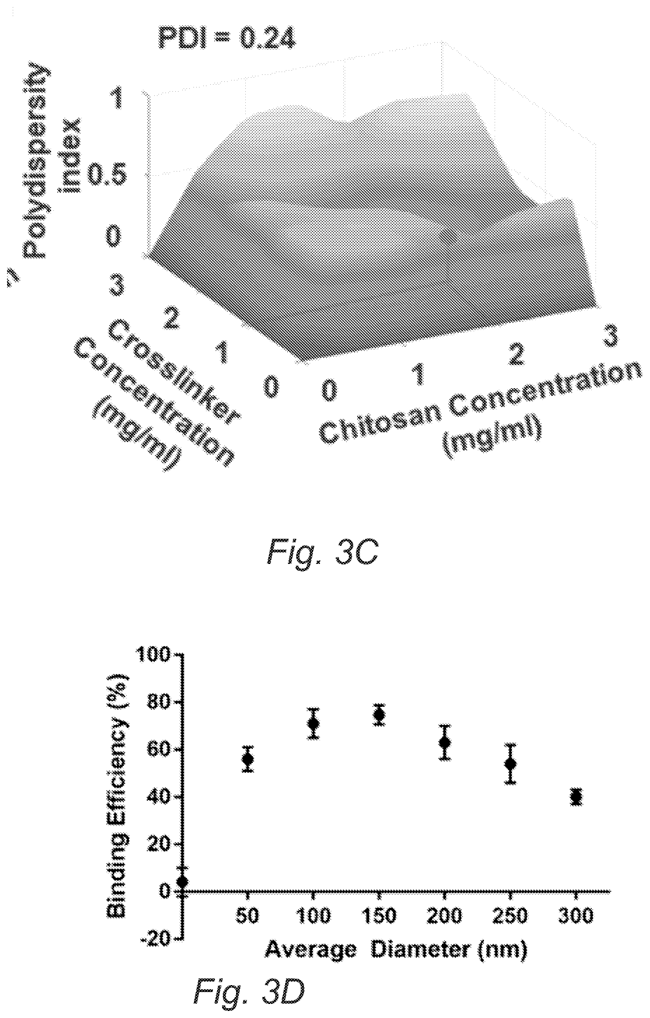

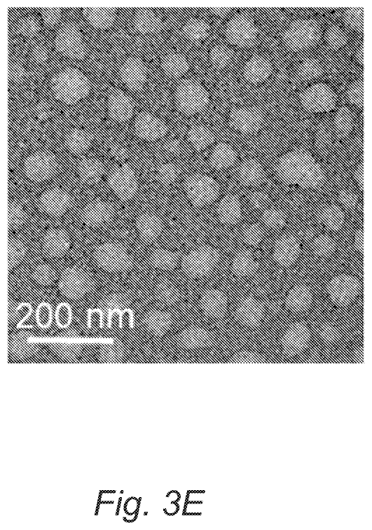

In previous studies, chitosan polymers have been shown to bind to mucins more effectively as the degree of deacetylation (DDA) increased [92], as the additional positively charged amino groups allow for increased interaction with negatively-charged sialic acid residues of mucin [93]. Similarly, in this study, upon deacetylation of the starting chitosan (before nanoparticle synthesis), the zeta potential of the chitosan solutions confirmed increasing % deacetylation increased the positive charge from 15.8±4.2 mV at 50% DDA to 27.3±2.8 mV at 90% DDA. When chitosan polymers of varying DDA (90%, 80%, 70%, and 50% DDA) were tested in a mucin binding assay, the highest DDA (90%) showed the greatest binding efficiency to mucin (75.1±5.0%) (FIG. 3A).

In order to identify the ideal nanoparticle diameter to adhere and diffuse through the mucosal layer, 90% DDA chitosan was used to synthesize chitosan nanoparticles (CS-NP) of various diameters (50, 100, 150, 200, 250, 300 nm), achieved by altering the starting concentrations of chitosan and poly-L-glutamic acid crosslinker during ionic gelation. Nanoparticle diameter has been found to affect the ability to bind and diffuse through mucus, as the mesh pore size (10-200 nm) of mucus sterically limits nanoparticles larger than 200 nm [94]. Chitosan concentrations were limited between 0.5 mg/ml and 3.0 mg/ml, as higher concentration ranges resulted in immediate aggregation of particulates upon dropwise addition to polyglutamic acid, and lower concentrations resulted in no nanoparticle formation. As shown in FIG. 3, CS-NP of approximately 150 nm in diameter demonstrated the highest mucin binding efficiency (74.8±4.0%) (FIG. 3D). We observe a decrease in binding for particles beyond 200 nm, which is consistent with the study by S. Bandi et al. demonstrating nanoparticles ≥200 nm in diameter had limited diffusivity through mucus due to mucin mesh steric hindrance [95]. Hence, we selected 90% DDA chitosan at 2 mg/ml and poly-Lglutamic acid crosslinker at 1 mg/ml synthesis conditions, which resulted in nanoparticles with the desired diameter of 150 nm and a low PDI of 0.24, to proceed with further studies (FIG. 2B, C, E). Increasing mucodiffusion has also been a viable engineering strategy to access underlying enterocytes and increase systemic bioavailability [96]. While not investigated in this study, the mucodiffusive properties of chitosan may play a role in navigating the GI tract, and will be evaluated in future studies.

### 1.2.2. Drug Release and Degradation Properties of CS-NC

To successfully deliver substances through the GI tract, enteric delivery systems must protect the payload from degradation and premature release in the low pH environment of the stomach [97]. The amino groups in chitosan (pKa=6.5) are protonated to form NH3+ in low pH (pH 1.2-2.5), providing strong electrostatic attractions to the oppositely-charged poly-glutamic acid crosslinker, which allows the particles to remain compacted and remain at their original size and retain the payload [98]. At higher pH values (pH=6.5-7.4), the amine groups of chitosan exist mostly in the NH2 form [99]. As a result, the electrostatic interactions between CS and crosslinker are weakened, which favors dissolution [100].

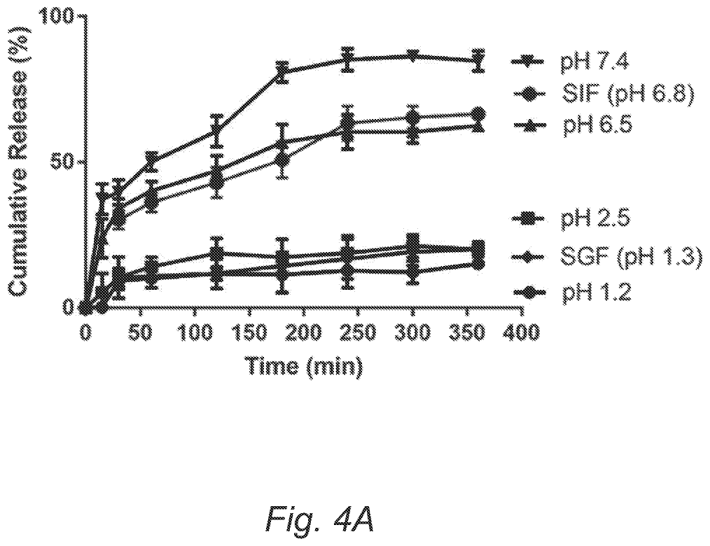

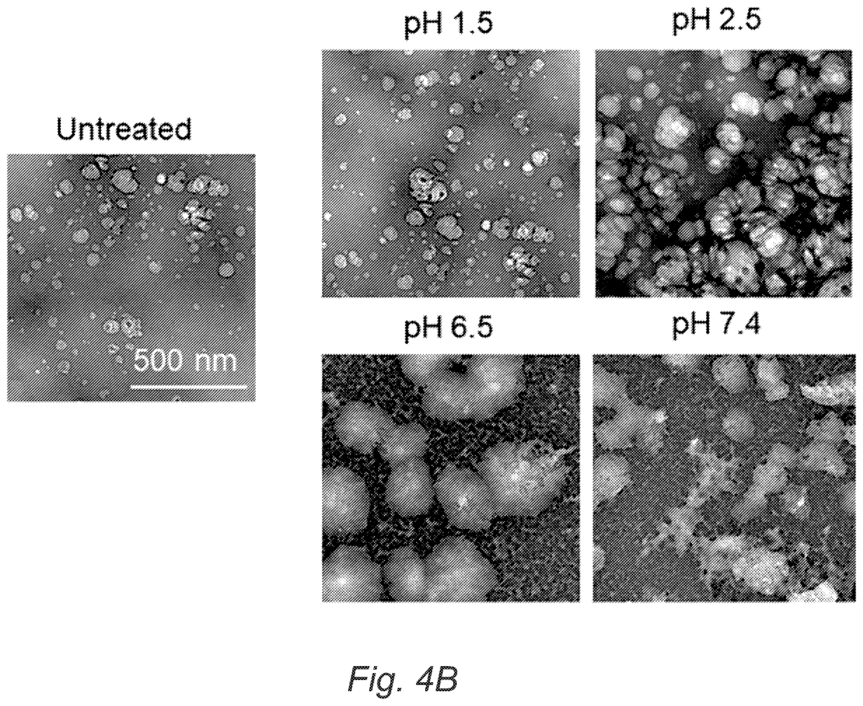

To verify CS-NP have the ability to protect drugs past the gastric environment, we tested met-loaded CS-NP (CS-NP met) in pH environments representative of a fasting stomach (pH 1.5), fed stomach (pH 2.5), duodenum of the small intestine (pH 6.5), circulating blood (pH 7.4), SGF (pH 1.3), and SIF (pH 6.8) [101,102]. The loading efficiency and loading capacity of met into CS-NP was determined to be 32.2±2.8% and 37.3±3.6%, respectively, likely due to the electrostatic interaction of positively charged met with the negatively charged crosslinker during nanoparticle synthesis [103]. To assess met release profiles, we quantified free met release from CS-NP met in the various pH environments for up to 6 h. We observed that payload release did not exceed 25% at pH conditions 1.2, SGF (pH 1.3), and 2.5, while greater than 50% release was found in pH 6.5, SIF (pH 6.8), and 7.4 at 3 h (FIG. 4A). Consistent with release studies, TEM images showed minimal morphological change at pH 1.2 and 2.5, (FIG. 4B), while an increase in diameter (˜250 nm) was seen in pH 6.5. At pH 7.4, CS-NP met lost their spherical morphology and fused with adjacent particles. In agreement with observations in other chitosan nanoparticle studies, water is able to intercalate through pores in the polymer matrix, expanding the particle and causing swelling and degradation [104]. This further suggests that CS-NP can remain stable and protect drugs under the low pH conditions found in the stomach, but swell and release drugs upon reaching neutral pH found in the small intestine and systemic circulation.

### 1.2.3 In Vitro Penetration Across Intestinal Epithelium

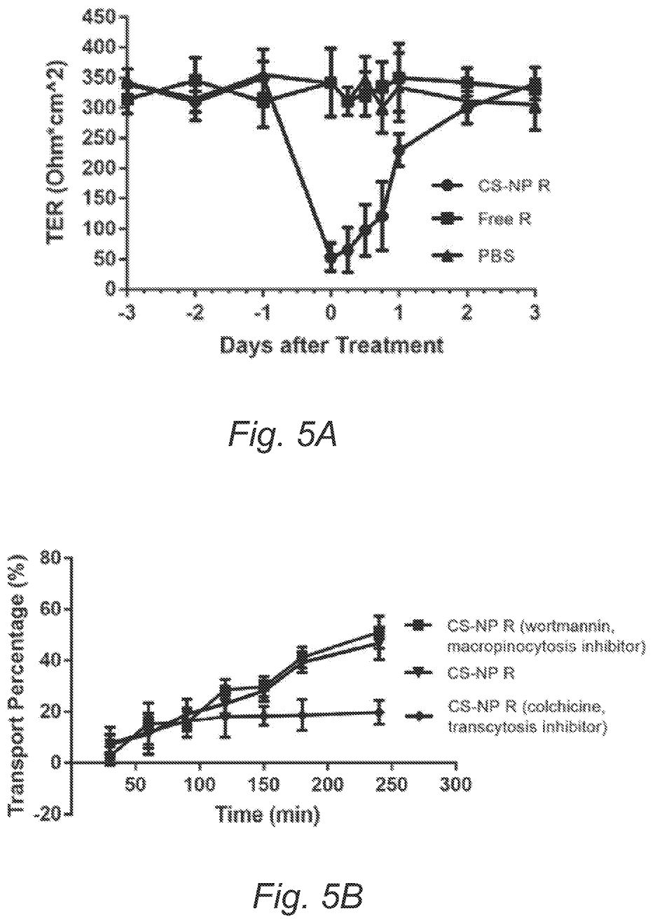

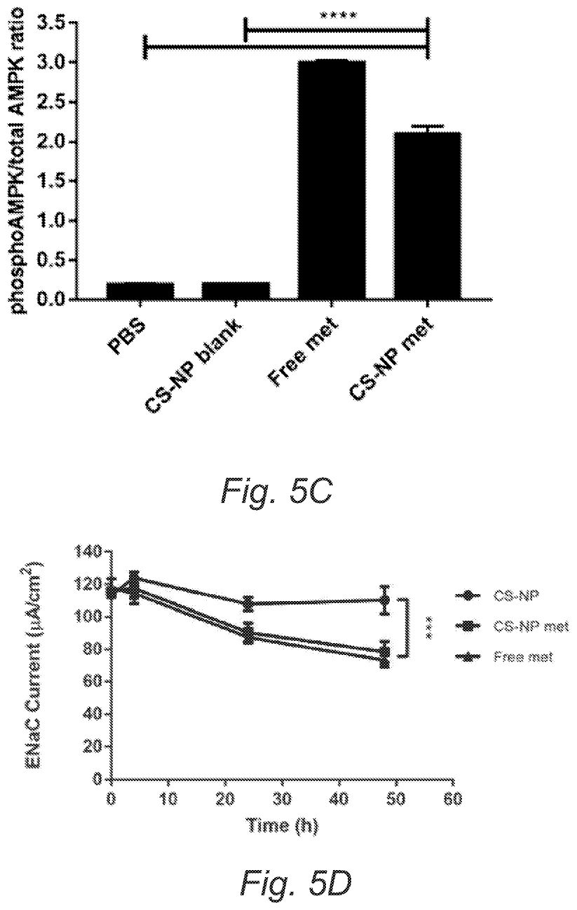

In addition to mucoadhesive properties and protection of payloads in the acidic environment of the stomach, chitosan has been suggested to enhance the penetration of the intestinal epithelial cell barrier by opening tight junctions and increase transport of luminal peptides, nutrients, and nanoparticles [105]. To verify this, CS-NP rhodamine (CSNP R), free rhodamine, or PBS was tested in an intestinal epithelial barrier model consisting of human colorectal Caco-2 cells, on Transwell membranes [106], and tight junction integrity was determined via transepithelial resistance (TER) measurements [79]. TER measurements were made three days before treatment, and again after administration of CS-NP R, free rhodamine, or PBS for up to 3 days. No changes in TER occurred in the PBS or free rhodamine-treated groups, while an 84.8% reduction in resistance to 53.1±32.3 Ohm*cm² was observed for CS-NP R 6 h after administration (FIG. 5A). A recovery to pretreated baseline resistance levels (355 Ohm*cm²) was seen after 3 days, suggesting the effects on tight junctions are reversible, yet persist enough on the time scale that digestion occurs in the human gut [107].

In addition to paracellular transport, transcellular transport has been reported for nanoparticles passage through the intestinal lining [108]. Previous studies have suggested chitosan nanoparticles can undergo endocytosis (clathrin-mediated) and macropinocytosis in intestinal cells [109]. Specifically, to determine if transcytosis or macropinocytosis is mainly responsible for the transport of CS-NP, Caco-2 cell layers were treated with colchicine (10 μM, for 60 min), wortmannin (0.06 mM, for 3 h), or no inhibitor before 100 μM of CS-NP R incubation, and 1205 rhodamine fluorescence was measured in the basolateral chamber over the course of 4 h. A 27% reduction of transport was seen when the transcytosis inhibitor colchicine was administered, while no reduction was seen when the macropinocytosis inhibitor wortmannin was administered (FIG. 5B). These initial findings suggest that transcytosis is a major pathway by which CS-NP are transported across intestinal epithelial layers [79,110,111], in addition to increasing paracellular permeation. Importantly, over 90% of cells were viable upon treatment with CS-NP as indicated by an MTS assay, suggesting transport differences are not due to cytotoxic effects of chitosan. The CS-NP R transport behavior is also distinct from free rhodamine administered in the same conditions. No statistically significant reduction is observed in transport of free R between the colchicine and wortmannin conditions, while colchicine reduces CS-NP R transport.

### 1.2.4 In Vitro Therapeutic Efficacy of Metformin-Loaded CS-NC

Upon characterizing the biomaterial properties of CS-NP and verifying its potential to deliver drugs through intestinal epithelia, met was loaded into CS-NP for ADPKD applications. We selected met, a first-line therapy already approved for diabetes, due to its secondary benefits in inhibiting ADPKD preclinically, resulting in several ongoing clinical trials repurposing met for ADPKD including METROPOLIS (NCT03764605) and TAME (NCT02656017) [112,113]. Specifically, met activates the 5′ AMP-activated protein kinase (AMPK) pathway by phosphorylating AMPK. This leads to inhibition of the mammalian target of rapamycin (mTOR) pathway [114], responsible for the expansion of cysts due to the over proliferation of renal tubular cells. Additionally, met inhibits intracellular generation of cAMP via inhibition of adenylyl cyclase [115], a key signaling pathway that drives cystogenesis in ADPKD. In addition, AMPK activation has been found to inhibit fluid secretion into cysts by inhibiting the cystic fibrosis transmembrane receptor (CFTR) channel [116], the key apical membrane chloride secretory route in ADPKD [117]. In ADPKD preclinical murine studies, met was administered at a dose of 300 mg/kg/day which greatly exceeds the dose currently prescribed for patients with diabetes (maximum 37.5 mg/kg/day). Even at low doses, 25% of patients already suffer from GI discomfort and approximately 5% are unable to tolerate met entirely due to these side effects [20-22]. Since the bioavailability of orally taken met is only 40%, it is expected that high doses are needed for PKD efficacy, which may further exacerbate the incidence of side effects [71]. To enhance the bioavailability of met for oral delivery in ADPKD, met was loaded into chitosan nanoparticles (CS-NP met), and the size and charge of CS-NP met was found to be unaltered compared to unloaded CS-NP (Table 1.2).

TABLE 1.2
Size and charge of unloaded CS-NP compared to CS-NP met.

| | Diameter (nm) | Zeta Potential (mv) |
|---|---|---|
| CS-NP | 145.5 ± 8.2 | +27.3 ± 2.8 |
| CS-NP met | 144.3 ± 6.6 | +27.8 ± 2.1 |

To first test therapeutic potential in vitro, mpkCCDc14 cells, derived from the cortical collecting duct, were treated with 300 μM met in CS-NP met, free met, unloaded CS-NP, or PBS for 12 h and phosphorylated (active) AMPK to total AMPK ratio was measured using an ELISA assay. As shown in FIG. 5C, an increase in phosphorylated AMPK to total AMPK ratio in both met-containing groups was seen: 3.0±0.1 for free met and 2.1±0.1 for CS-NP met (p<0.005), while no change was found upon PBS and CS-NP blank treatment. While the free drug showed higher therapeutic efficacy at the same dose, we believe this is due to the slow release profile of met from CS-NP at 7.4 pH (FIG. 4A), compared to the bolus effect of free met. Additionally, the effect of CS-NP met on the reduction of ENAC current, a measure of the CFTR activity, was analyzed. After 15 min, 3 h, 24 h, and 48 h of CS-NP met, free met, CSNP blank, or PBS treatment, the CS-NP met and free met groups showed a marked decrease in ENaC current in mpkCCDc14 cells. The largest change was seen after 48 h, with CS-NP met reaching 81.1 μA/cm2 and free met at 79.2 μA/cm2, while unloaded CS-NP ENaC remained at the 117.2 μA/cm2 baseline (FIG. 5D, p<0.005). Taken together, these studies confirmed the therapeutic efficacy of CS-NP met was not hindered, as it produced similar AMPK activity and ENaC inhibition compared to free met. As over 90% of cells were viable upon treatment with CS-NP when assayed by MTS, the observed ENaC and AMPK changes are not due to cytotoxic effects of chitosan.

### 1.2.5. Ex Vivo Imaging of CS-NP In Vivo and Intestinal Localization

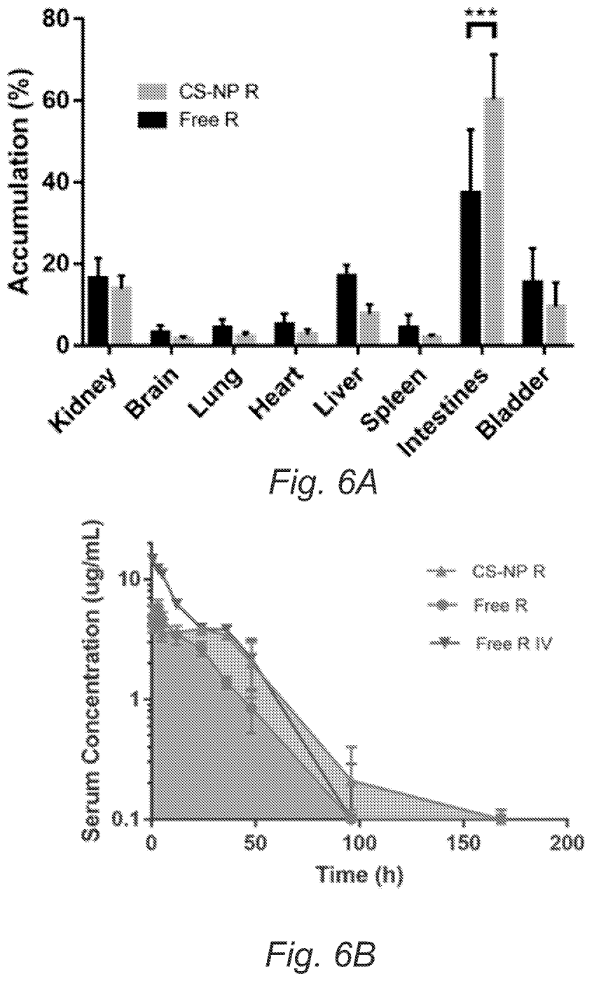

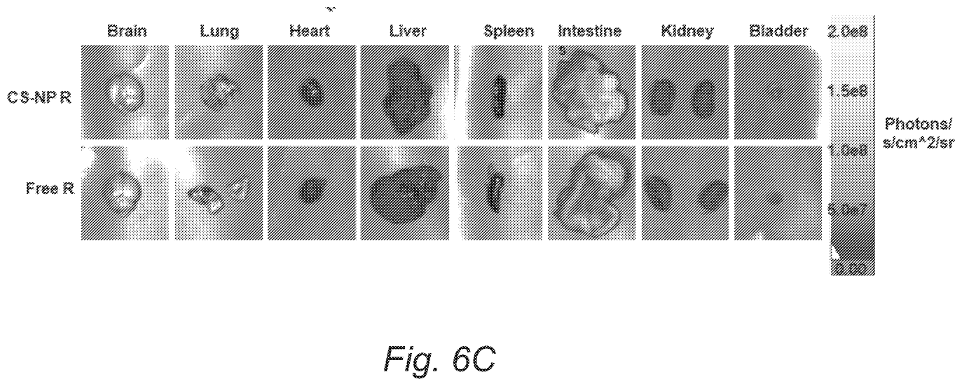

Next, to assess the ability of CS-NP to enhance drug bioavailability via oral delivery in vivo, 10 mg/kg rhodamine was first encapsulated into CS-NP (CS-NP R) and C57BL/6 J mice were orally gavaged with 200 μL CS-NP R or free R, and after 24 h, ex vivo imaging was conducted. As shown in FIG. 6, ex vivo optical imaging demonstrated the majority of CSNP R and free R accumulated in the intestines, liver, kidneys, and bladder, and upon quantitative analysis, CS-NP R showed 60.3±11.0% of total organ fluorescence accumulation in the intestines vs. 37.4±15.5% for free R (p<0.005, FIG. 6A). A time course for ex vivo imaging using CS-NP R at 3 h and 48 h showed similar trends: at 3 h, CS-NP R had 42 0.1±12.0% accumulation while free R had 45.8±14.0% (p<0.005). At 48 h, 52.3±10.1% accumulation was found for CS-NP R in the intestines whereas 38.5±15.1% accumulation for free R. Notably, serum fluorescence showed a higher area under the curve (AUC) ratio of 1.3:1 for CS-NP R compared to free R over the course of 7 days (FIG. 6B), demonstrating enhanced depot to systemic circulation.

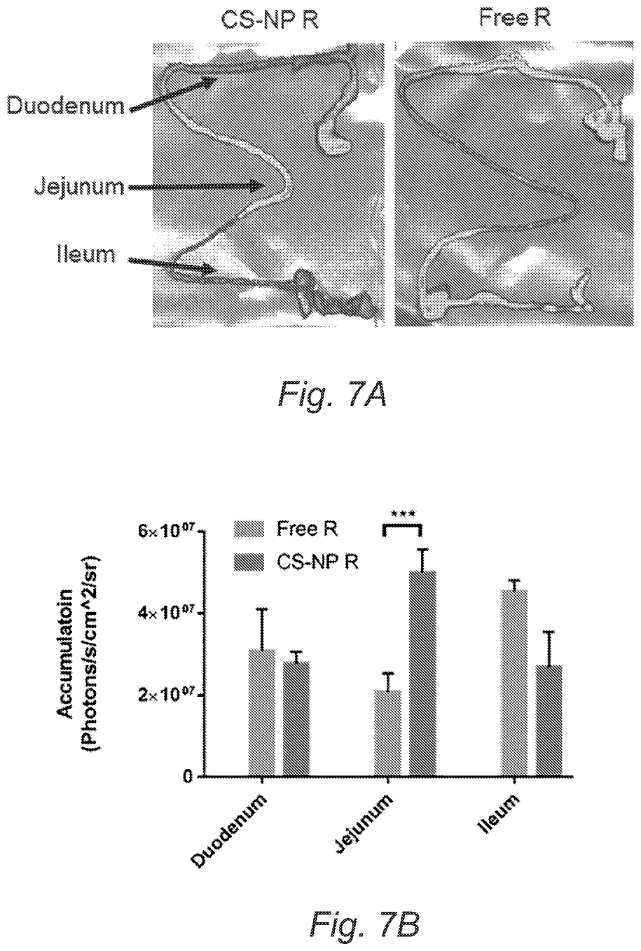

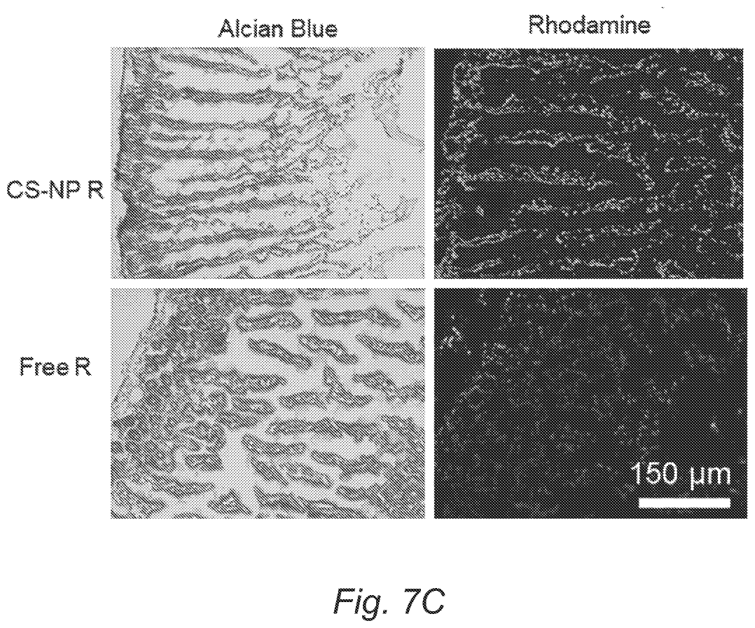

Upon further assessment of CS-NP R localization within the intestines via ex vivo imaging, CS-NP R was found adhered to the jejunum of the intestines (FIG. 7A, B). Fluorescence microscopy of intestinal sections also confirmed higher rhodamine signal in the jejunum as well as higher colocalization of CS-NP R with mucin vs. free R (FIG. 7C). This is beneficial for oral delivery as the Peyer's patches located within the jejunum, as well as the larger surface area compared to the duodenum and ileum, are responsible for the majority of nutrient uptake, as well as facilitating nanoparticle transport into systemic circulation [118,119]. Overall, these finding suggests that through mucoadhesion, CS—NP is retained in the jejunum which allows for sustained drug release and bioavailability Moreover, no morphological differences were found in other organs between treatment and PBS control histologically, confirming safety of CS-NP.

### 1.2.6 Therapeutic Efficacy of CS-NC Met in PKD Mice

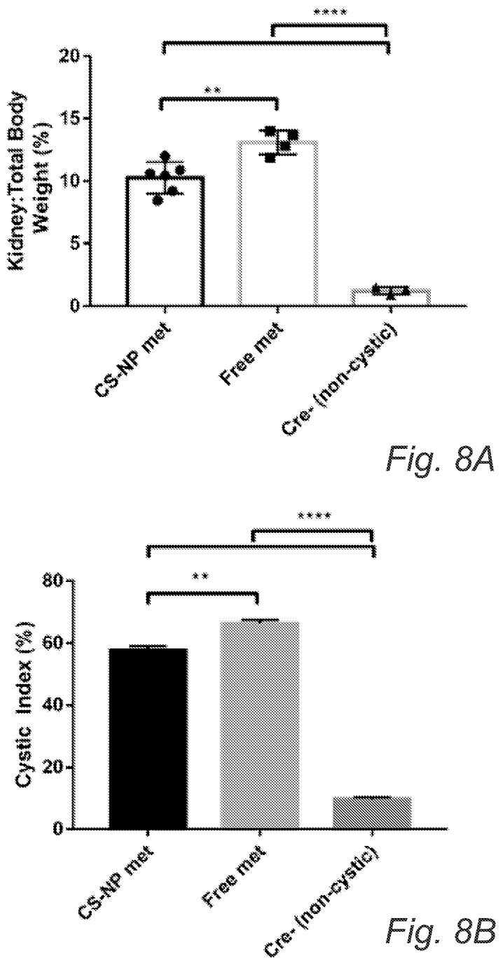

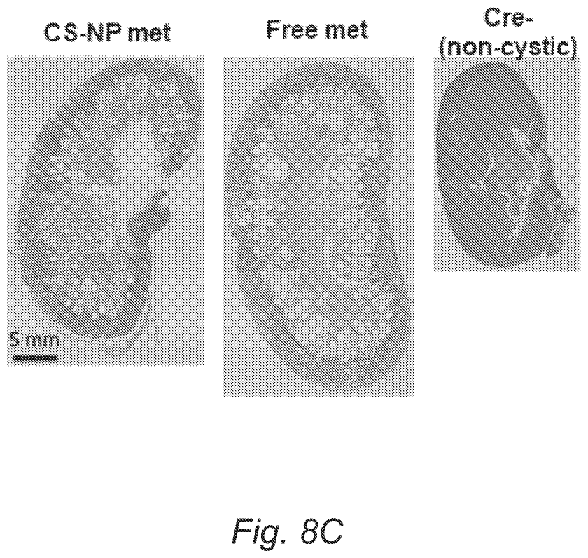

To confirm the viability of CS-NP to act as an oral delivery vehicle in chronic diseases, CS-NP met was administered in the ADPKD murine model, Pkd1fl/fl; Pax8-rtTA; Tet-O cre [120]. In this model, a rapidly progressing PKD phenotype can be developed by knockout of the PKD1 gene, induced by doxycycline injection on P10 and P11. Then starting on P12, mice were orally gavaged with 300 mg/kg of met loaded in CS-NP met, control CS-NP, or free met every two days and euthanized on P22 when a severe cystic phenotype is expected. Kidneys were excised to assess kidney to body weight (KW/BW) ratio and stained with H&E to compare cystic index. In CS-NP met-treated mice, a greater decrease in the KW/BW ratio was found compared to free met (10.3±1.1 vs. 13.1±1.0, p≤0.01), confirming enhanced therapeutic efficacy in slowing of cystogenesis of met when delivered via CS-NP (FIG. 8A). Moreover, cystic index was statistically lower in CS-NP met-treated mice compared to mice treated with the free drug (57.6±1.2% vs. 66.5±0.8%, p≤0.01, FIG. 8B, C). A Cre-mouse serves as healthy control in which the PKD1 gene knockout is not activated and Cre-kidneys represent normal kidney morphology.

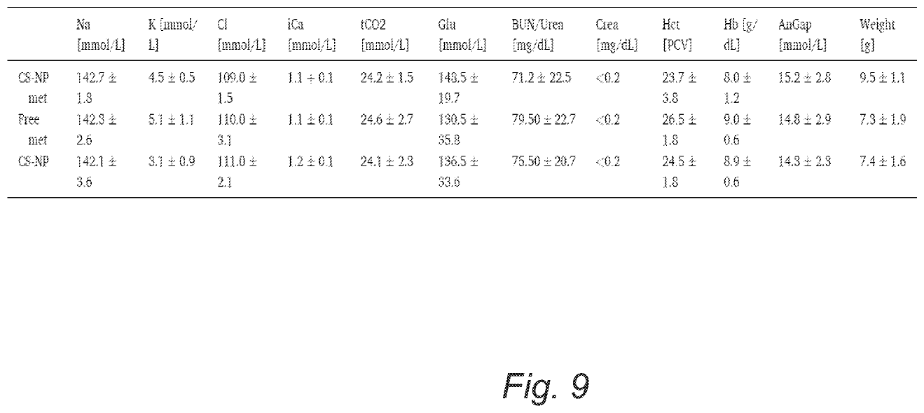

Although a met dose of 300 mg/kg daily has been found to activate AMPK in previous murine models [20], it is higher than what is currently prescribed for patients with diabetes (maximum 37.5 mg/kg/day). Our study administered 300 mg/kg met every two days instead, and confirmed efficacy via oral delivery that was comparable to previous IP delivery studies [20]. Future dose de-escalation studies will be conducted to examine the full benefits of CS-NP in increasing therapeutic efficacy without compromising safety. Regarding renal biocompatibility, kidney health markers including blood urea nitrogen, creatinine, and serum electrolytes were found to remain similar between treatment groups, demonstrating CS-NP formulations do not cause kidney damage (Table 1.1, FIG. 9). The BUN levels correspond to mildly impaired renal function expected in polycystic kidney mice, on the order of 40-80 mg/dL [121]. In sum, CS-NP demonstrated a higher therapeutic efficacy when compared to free drug at the same dose, and is a safe platform that can overcome the physiological barriers of oral delivery. Uniquely, this is the first nanoparticle delivery platform for ADPKD, and our study highlights CS-NPs as a viable oral delivery platform for chronic conditions.

### 1.3. Conclusion

Chitosan nanoparticles (CS-NP) were investigated as a promising drug delivery platform for oral delivery in chronic kidney disease. CSNPs were synthesized through ionic gelation and their physiochemical properties were characterized. In vitro, CS-NPs demonstrated effective mucoadhesion, while protecting premature release of the payload in the low pH environment of the stomach. When met-loaded CS-NP were cultured with cells in vitro, therapeutic efficacy was found via AMPK activation and ENaC current reduction. Moreover, upon oral gavage in a murine model of PKD, disease burden was significantly reduced upon met delivery using CS-NP compared to the free drug. While free met at the dosages used in the study did not cause significant toxicities, future studies assessing high met dosages or increasing dose exposure in the slowly developing mouse model of PKD that more closely mimics the chronic nature of the human disease will more fully elucidate the benefits of delivering drugs in the CS-NP system. Furthermore, the observed advantage of CS-NP in increasing systemic delivery may also be more evident upon loading candidate ADPKD drugs with poor oral bioavailability, such as somatostatin or bardoxolone methyl. Our study provides the framework to advance chitosan nanotechnology for PKD; future studies will include additional animal models including slowly progressing PKD models to further mimic the chronic nature of PKD, as well as large porcine animal models as we look towards clinical translation.

### 2. The Effect of Size, Charge, and Peptide Ligand Length on Kidney Targeting by Small, Organic Nanoparticles

### 2.1 Introduction

To study how size, charge, peptide ligand length, and surface ligand density affects nanoparticle kidney-targeting ability and to optimize the kidney-targeting potential of PAMs, we developed a library of micelles by incorporating variations of the original zwitterionic (KKEEE)₃K peptide including (KKEEE)₂K, KKEEEK, (EEKKK)₃E, (EEKKK)₂E, EEKKKE, KKKKK, and EEEEE. The size and surface charge of the micelle library was characterized by TEM, DLS, and zeta potential, and to assess nanoparticle kidney-targeting ability, micelles were intravenously administered into C57B/6J mice. Kidney targeting and micelle biodistribution was evaluated via ex vivo imaging after 24 hours. Moreover, micelle targeting to megalin was evaluated through immunohistochemistry, and kidney biocompatibility was assessed through histology, blood urea nitrogen, and urine creatinine.

### 2.2 Results and Discussion

### 2.2.1 Synthesis and Characterization of PAMs

The kidney-targeting peptide (KKEEE)₃K was found to accumulate in the kidneys in part by binding to the megalin receptor expressed on proximal tubule cells and is composed of three repeats of the peptide sequence, KKEEE.¹³ Previously, when (KKEEE)₃K was incorporated into micelles and assessed in vivo, their biodistribution profile showed enhanced kidney accumulation but also liver accumulation of 35% likely due to the MPS system as (KKEEE)₃K PAMs were 15 nm in diameter and larger than the cut off reported for passage through the GFB (8-10 nm).¹³ To optimize the physicochemical properties of PAMs, herein, we synthesized micelles with fewer repeats of KKEEE to test how peptide repeat number affected kidney accumulation. Moreover, since micelles are self-assembled from monomers containing DSPE hydrophobic tails linked to polyethylene glycol (PEG), PEG molecular weight was also varied to further study the size effects on the renal targeting ability of nanoparticles. In addition to peptide repeat number and size, peptide sequences of opposite charge (e.g., EEKKK) as well as non-zwitterionic peptide sequences (e.g., KKKKK and EEEEE) were incorporated to test the charge selectivity of the GFB on micelles. Furthermore, to assess how peptide ligand surface density affects renal targeting ability, we synthesized micelles with 50% or 100% peptide surface presentation.

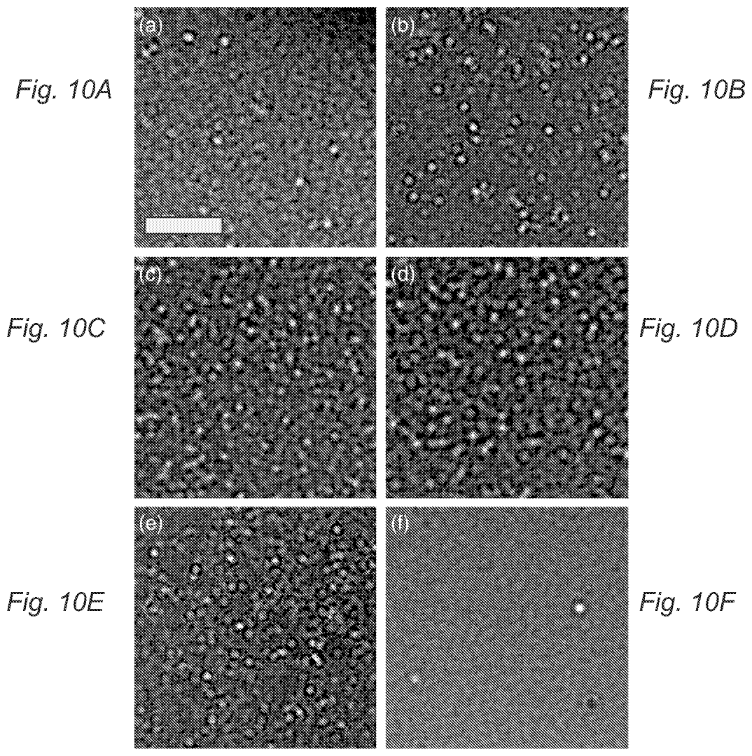

To characterize the micelles, PAMs were self-assembled in water or PBS at 100 μM (which is above the critical micelle concentration of 1 μM33) and assessed via TEM and DLS. TEM images demonstrated spherical morphology of all PAMs (FIG. 10), and DLS confirmed that decreasing the number of KKEEE repeats on nanoparticles correlated with a slight decrease in hydrodynamic diameter, although the diameter of all micelles were close in range to one another: PEG2000-(KKEEE)₃K (16.6±2.5 nm), PEG2000-(KKEEE)₂K (12.2±0.6 nm), PEG2000-(KKEEE)K (10.6±1.7 nm); PEG2000-(EEKKK)₃E (12.4±1.2 nm), PEG2000-(EEKKK)₂E (14.0±1.8 nm), PEG2000-(EEKKK)E (11.8±1.5 nm). PEG2000-KKKKK and PEG2000-EEEEE had an average diameter of 11.2±1.2 nm and 10.2±1.3 nm, which were similar to PEG2000-(KKEEE)K and PEG2000-(EEKKK)E due to the shorter peptide sequence (Table 2.1). As expected,¹²³ increasing the PEG molecular weight to 5,000 resulted in a slight increase in diameter of nanoparticles to 17.2±1.8 nm, and nanoparticles consisting of DSPE-PEG1000 had a diameter of 10.4±1.8 nm (Table 2.1).

Interestingly, despite (KKEEE)₃K and (EEKKK)₃E having an opposite net charge of −2 and +2 derived from additional negatively charged glutamic acid (E) or positively-charged lysine (K), the zeta potential of both micelles were found to be near neutral at −0.6±1.4 mV and 0.03±0.1 mV, respectively (Table 2.1). Similarly, the zeta potentials of PEG2000-(KKEEE)₂K (net −1), PEG2000-(EEKKK)₂E (net+1), PEG2000-(KKEEE)K (net 0), and PEG2000-(EEKKK)E (net 0) were also found to be near neutral at −0.2±0.3 mV, −3.1±6.4 mV, −1.1±0.6 mV, and −0.1±0.7 mV. Since all these micelles consisted of 50:50 molar ratio of peptide-DSPE-PEG2000 to DSPE-PEG2000-methoxy, it is possible that the near-neutral charge of DSPEPEG2000-methoxy amphiphiles masked any charge differences of the micelles resulting in neutral zeta potentials. As the zeta potential of DSPE-PEG2000-NT (consisting of 100% DSPE-PEG2000-methoxy) was found to be near neutral (−3.2±5.4 mV), the addition of 50% molar ratio of DSPE-PEG2000-methoxy may have affected their surface charge, leading to the resultant near-neutral zeta potential measurements of PEG2000-(KKEEE)₃K and PEG2000-(EEKKK)₃E. This was also the case with nanoparticles consisting of 50% KKKKK, which had a zeta potential of 0.07±0.2 mV (Table 2.1). On the other end, when the peptide sequence was altered to 50% EEEEE, micelles consisted of a negative zeta potential of −40.9±4.6 mV.

TABLE 2.1
Characterization of micelles by DLS and zeta potential

| | Peptide | | Zeta | Zeta |
|---|---|---|---|---|
| | surface | Diameter | potential | potential (mV) |
| NP | density (%) | (nm) | (mV) | Net charge |
| PEG2000- | 50 | 16.6 ± 2.5 | −0.6 ± 1.4 | −2 |
| (KKEEE)₃K | | | | |
| PEG2000- | 50 | 12.2 ± 0.6 | −0.2 ± 0.3 | −1 |
| (KKEEE)₂K | | | | |
| PEG2000- | 50 | 10.6 ± 1.7 | −1.1 ± 0.6 | 0 |
| (KKEEE₂K | | | | |
| PEG2000- | 50 | 12.4 ± 1.2 | 0.03 ± 0.1 | +2 |
| (KKEEE)₃K | | | | |
| PEG2000- | 50 | 14.0 ± 1.8 | −3.1 ± 6.4 | +1 |
| (EEKKK)2E | | | | |
| PEG2000- | 50 | 11.8 ± 1.5 | −0.1 ± 0.7 | 0 |
| (EEKKK)E | | | | |
| PEG2000- | 50 | 11.2 ± 1.2 | 0.1 ± 0.2 | +5 |
| KKKKK | | | | |
| PEG2000- | 50 | 10.2 ± 1.3 | −40.9 ± 4.6 | −5 |
| EEEEE | | | | |
| PEG2000- | 100 | 10.6 ± 0.4 | −41.4 ± 2.9 | −2 |
| (KKEEE)₃K | | | | |
| PEG2000- | 100 | 15.0 ± 0.4 | 14.3 ± 1.6 | +2 |
| (EEKKK)₃E | | | | |
| PEG1000- | 50 | 10.4 ± 1.8 | −2.9 ± 2.9 | −2 |
| (KKEEE)₃K | | | | |
| PEG5000- | 100 | 17.2 ± 1.8 | −9.6 ± 1.0 | −2 |
| (KKEEE)3K | | | | |
| PEG2000-NT | 100 | 7.5 ± 0.1 | −3.2 ± 0.8 | 0 |

Given these results, we synthesized micelles consisting of entirely PEG2000-(KKEEE)₃K and PEG2000-(EEKKK)₃E. Zeta potential measurements of 100% PEG2000-(KKEEE)₃K and 100% PEG2000-(EEKKK)₃E micelles were found to be −41.4±2.9 mV and 14.3±1.6 mV, which is more consistent with the net charge of the peptide sequence, −2 and +2, respectively (Table 2.1).

### 2.2.2 In Vivo Renal Targeting and Biodistribution

To test the effects of nanoparticle characteristics on renal targeting, Cy7-labeled micelles were intravenously administered into 6-7 week old male and female C57B/6 J mice and after 24 hours, micelle accumulation was assessed via ex vivo imaging of the kidneys, brain, lung, heart, liver, spleen, intestine, and bladder. 10 mol % of Cy7 was included into micelles to maximize the fluorescence signal without quenching and micelles with 45:45:10 molar ratio of DSPE-PEG-methoxy: DSPE-PEG-peptide:DSPE-PEG-Cy7 or 90:10 molar ratio of DSPEPEG-peptide:DSPE-PEG-Cy7 were synthesized for in vivo studies.¹²⁴ Despite all micelles being at or above the reported renal filtration cut-off size, all micelles accumulated in the kidneys to a greater extent than all other organs with the exception of 90% PEG5000-(KKEEE)₃K.125 90% PEG5000-(KKEEE)₃K, which had the highest PEG molecular weight and largest diameter of 17.2±1.8 nm (Table 2.1), mostly accumulated in the liver (1.6×10⁹±1.3×10⁸ p/s/cm²/sr) likely via recognition of the MPS system. However, this was not statistically significant with its accumulation in the kidneys, which indicates an ability to simultaneously pass through the GFB and accumulate in the kidneys. Although the size of micelles was slightly larger than the cut-off diameter of glomerular filtration (10 nm), kidney accumulation and passage through the GFB has been found for 60-100 nm polycation-siRNA nanoparticles and other soft macromolecules with the diameter beyond the cut-off size of 8-10 nm.^{123,126}

2.2.2.1 Biodistribution of Micelles with Varying Number of Peptide Repeats

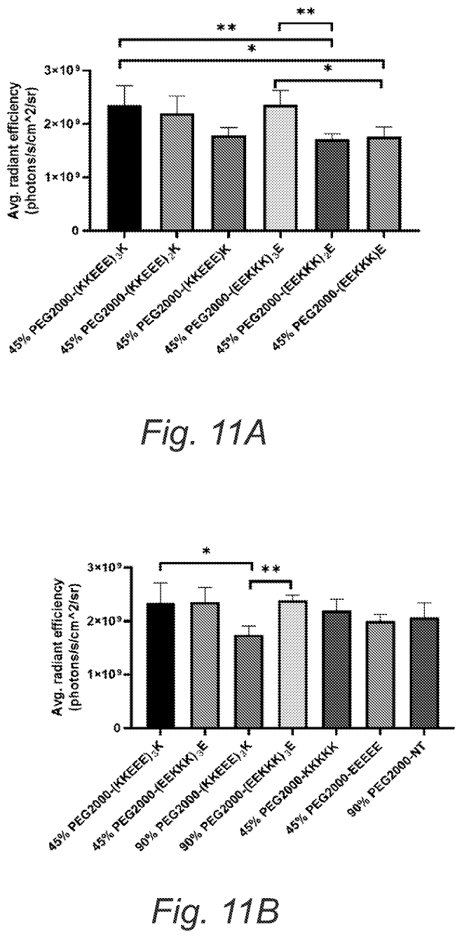

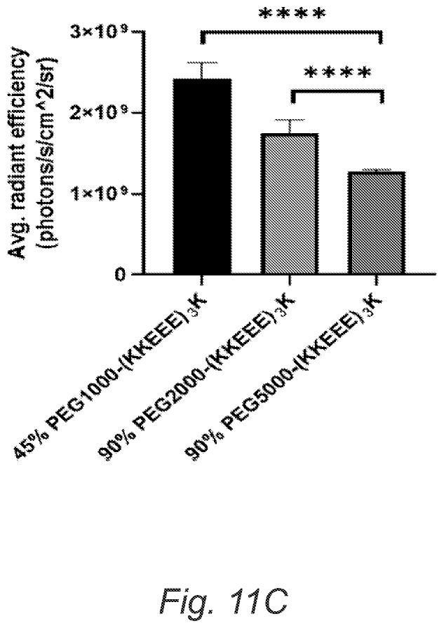

In FIG. 11A, the effect of KKEEE/EEKKK repeats on nanoparticle kidney targeting in vivo was evaluated. Although we hypothesized that peptides with fewer repeats of KKEEE/EEKKK would decrease the overall molecular weight and size of micelles and thereby improve the ability of nanoparticles to pass through the GFB and accumulate in the kidneys, as shown in FIG. 11A, additional sequence repeats trended towards having higher renal accumulation. Specifically, for micelles with 45% peptide surface presentation, PEG2000-(KKEEE)₃K (2.3×10⁹±3.7×10⁸ p/s/cm²/sr) had the highest fluorescence intensity, followed by PEG2000-(KKEEE)₂K (2.2×10⁹+3.3×10⁸ p/s/cm²/sr) and PEG2000-(KKEEE)K (1.8×10⁹±1.5×10⁸ p/s/cm²/sr, FIG. 11A, although not statistically significant). Similarly, for PAMs with EEKKK repeats, PEG2000-(EEKKK)₃E (2.4×10⁹±2.7×10⁸ p/s/cm²/sr) showed higher renal fluorescence intensity than PEG2000-(EEKKK)₂E (1.7×10⁹+1.0×10⁸ p/s/cm²/sr, p<0.01). PEG2000-(EEKKK)E had a fluorescence intensity of 1.8×10⁹±1.9×10⁸ p/s/cm²/sr, which was also statistically lower compared to PEG2000-(EEKKK)₃E (p <0.05, FIG. 11A). Given that KKEEE and EEKKK are zwitterionic peptides, one possible explanation for the higher kidney accumulation of micelles consisting of PEG2000-(KKEEE)₃K and PEG2000-(EEKKK)₃E micelles is that the additional peptide repeats provided enhanced zwitterionic characteristics and hence, resistance to nonspecific protein and opsonin absorption as well as liver uptake via the MPS system.¹²⁷⁻¹³² Additionally, given that the hydrodynamic diameter of the micelles with varying peptide repeats fell within a narrow range (Table 2.1), peptide sequence and ligand length may play a more important role in the renal accumulation of micelles rather than size, which will be further probed in future studies.

2.2.2.2 Biodistribution of Micelles with Opposite Charge

In FIG. 11B, we compared the charge effects on micelle renal targeting. Despite having an opposite net charge of −2 and +2, PEG2000-(KKEEE)₃K and PEG2000-(EEKKK)₃E with 45% surface peptide density accumulated in the kidneys to a similar extent (2.3×10⁹±3.7×10⁸ p/s/cm²/sr and 2.4×10⁹±2.7×10⁸ p/s/cm²/sr, respectively, FIG. 11B). As found in Table 2.1, the zeta potentials of both micelles were near neutral due to the incorporation of PEG2000-methoxy, which likely masked any charge differences that contributed to kidney targeting. Moreover, as mentioned, both peptides are zwitterionic and nanoparticles coated with zwitterionic materials have been reported to resist serum protein adsorption in vivo due to the highly hydrophilic surface and anti-fouling properties.^{133,134} On the other hand, micelles consisting of 90% PEG2000-(KKEEE)₃K, which had zeta potential of −41.4±2.9 mV, had lower kidney accumulation (1.8×10⁹±1.7×10⁸ p/s/cm²/sr) than the positively-charged (14.3±1.6 mV) 90% PEG2000-(EEKKK)₃E (2.4×10⁹±1.0×10⁸ p/s/cm²/sr, p<0.05). These results correlated with findings by Liang et al. and Balogh et al. that reported the GFB is a charge-selective barrier in which positively charged nanoparticles pass through the GFB more easily than negatively charged nanoparticles and macromolecules due to electrostatic repulsion of the GBM.¹³⁵⁻¹³⁸ In addition, 90% PEG2000-(KKEEE)₃K had significant higher liver uptake (1.6×10⁹±5.0×10⁷ p/s/cm²/sr) than 90% PEG2000-(EEKKK)₃E (1.0×10⁹±9.9×10⁷ p/s/cm²/sr, p<0.0001). The higher liver uptake of negatively-charged 90% PEG2000-(KKEEE); K was similar to the results by Xiao et al that reported negatively-charged PEG-oligocholic acid based micellar nanoparticles (−26.9±1.7 mV) have higher liver uptake than positively-charged counterparts (3.6±0.8 mV) that were similar in size (18˜21 nm).¹³⁹

### 2.2.2.3 the Effects of PEG Molecular Weight and Size on the Biodistribution of Micelles

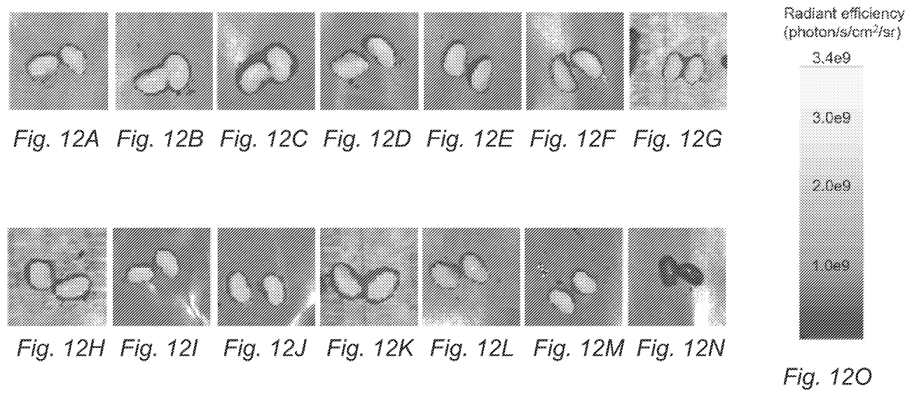

In FIG. 11C, we evaluated how the size of nanoparticles alters kidney targeting and compared micelles with varying PEG molecular weight.¹⁴⁰ (KKEEE)₃K micelles consisting of PEG1000 had renal accumulation of 2.43×10⁹+2.0×10⁸ p/s/cm²/sr that was significantly greater than (KKEEE)₃K micelles consisting of PEG5000 (1.28×10⁹±2.3×10⁸ p/s/cm²/sr, p<0.0001, as 45% PEG1000-(KKEEE)₃K micelles were found to be 10.4±1.8 nm in diameter and near the reported renal filtration cutoff size (Table 2.1, FIGS. 11C and 12). Similarly, PEG5000-(KKEEE)₃K showed lower renal accumulation compared to PEG20000-(KKEEE)₃K with 90% peptide density (1.28×10⁹+2.3×10⁸ p/s/cm²/sr and 1.8×10⁹+1.7×10⁸ p/s/cm²/sr, respectively, p<0.0001). Hence, our results demonstrate a negative correlation between the size and renal targeting ability of PAMs. The potential influence of the protein corona on micelles on GFB penetration will be further studied in the future to better understand the factors that affect nanoparticle kidney targeting.

2.2.3 Micelle Colocalization with Megalin

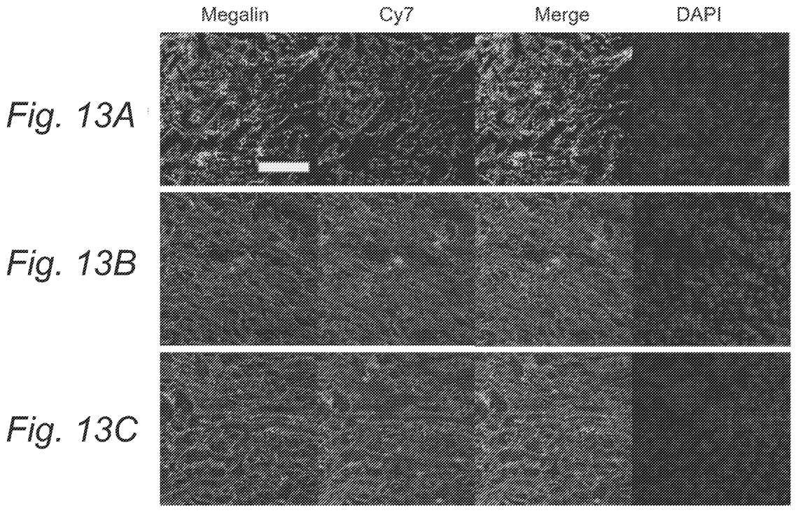

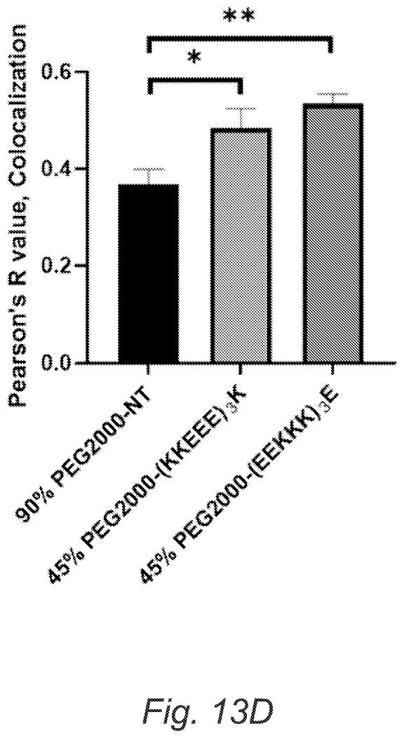

As mentioned, previously, the (KKEEE)₃K peptide was found to target the kidney in part through megalin, a multiligand receptor that is present on the plasma membrane of proximal tubule cells, and the (KKEEE)₃K peptide showed significantly reduced uptake in megalin deficient mice.^{19,20,141-143} As demonstrated by Vegt et al, megalin has been reported to associate with a library of peptides (octreotide, octreotate, minigastrin, exendin, and neurotensin) of varying charges.¹⁴⁴ To verify micelle binding to megalin, immunohistochemistry staining of kidneys of mice treated with 45% PEG2000-(KKEEE)₃K as well as 45% PEG2000-(EEKKK)₃E were assessed, and colocalization was calculated. A similar Pearson's R value, corresponding to colocalization, of 45% PEG2000-(EEKKK)₃E (0.53±0.02) to megalin was shown with 45% PEG2000-(KKEEE)₃K (0.48±0.04, not statistically significant) vs. the NT micelle (0.37±0.03, p<01, FIG. 13), confirming that in addition to (KKEEE)₃K PAMs, PAMs consisting of zwitterionic peptides of opposite charge also accumulate in the kidneys in part via megalin. This corresponds with the ex vivo results that showed 45% PEG2000-(EEKKK)₃E had similar kidney accumulation to 45% PEG2000-(KKEEE)₃K (FIG. 11A).

### 2.2.4 Tissue Morphology and Kidney Health

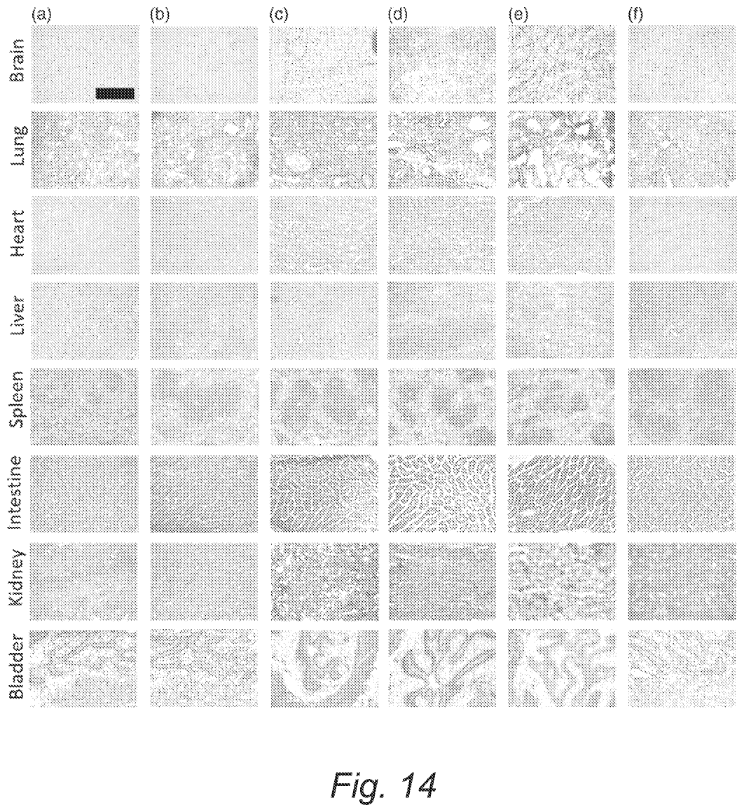

To assess the safety and biocompatibility of micelles and their eventual application as drug delivery vehicles, after ex vivo imaging, the brain, lung, heart, liver, spleen, intestine, kidney, and bladder were stained with H&E. In agreement with other PAM reports, the tissue morphology of all organs including the liver, intestine, and kidneys, where PAMs mostly accumulated, showed no tissue damage, and no significant difference was found to the PBS treatment group (FIG. 14).^{4,5,28,145,146} Kidney health and function of all the mice treated with micelles were further evaluated by assessing blood urea nitrogen (BUN) and urine creatinine levels. BUN levels for C57BL/6 mice with healthy kidney function is 25.0-75.0 mg/dl,¹⁴⁷ and all the groups in this study were found to have BUN levels within this healthy range (Table 2.2). Similarly, the creatinine concentration in the urine indicates kidney health and urine creatinine levels for all groups were measured and found to fall within the healthy range for C57BL/6 mice (4.7±3.1 mg/dl, Table 2.2).¹⁴⁸

### 2.2.3 Conclusion

In the experiments of this section, the physicochemical properties of micelles were altered by developing a library of micelles containing various peptide sequences and molecular weight and assessed its effect on renal targeting. Micelles were first characterized by TEM and DLS before intravenous administration into wild-type mice. After 24 hours post-administration, ex vivo imaging was conducted to compare kidney targeting ability. All micelles tested in this study had high kidney accumulation, and our studies showed that nanoparticles with more zwitterionic amino acid repeats that are positively charged and close to the renal filtration cut-off size tend to have higher renal accumulation. Furthermore, histological analyses confirmed no tissue damage, and kidney function levels of mice were within normal ranges. Overall, our studies indicate the potential of micelles as targeting nanocarriers for kidney applications.¹⁴⁹ Moreover, the therapeutic efficacy of drug-loaded micelles and the contribution of nanoparticle delivery through the peritubular capillaries will be assessed in CKD mouse models.

### 2.2.4. Materials and Methods

### 2.2.4.1 Micelle Synthesis

Peptides were synthesized using standard Fmoc-mediated solid-phase peptide synthesis on an automatic PS3 peptide synthesizer (Protein Technologies, Tucson, AZ) with rink Amide resin (Protein Technologies, Tucson, AZ). A cysteine was added to the N-terminus of all the peptide sequences in order to make a thioether linkage reaction. The peptides were then cleaved from the resin with 94:2.5:2.5:1 volume ratios of trifluoroacetic acid: 1,2-ethanedithiol: H2O:triisopropylsilane. Cleaved peptides were precipitated and washed several times with ice cold diethyl ether, dissolved in Milli-Q water, lyophilized, and stored at −20° C. The crude peptides were purified by reverse-phase high performance liquid chromatography (HPLC) (Shimadzu, Kyoto, Japan) on a C18 column (Phenomenex, Torrance, CA) at 55° C. with 0.1% formic acid in acetonitrile/water mixture. The purified peptides were characterized using matrix-assisted laser desorption ionization time-of-flight mass spectral analysis (MALDI-TOF) (Autoflex speed, Bruker, Billerica, MA) and conjugated to 1,2 distearoyl-sn-glycero-3-phosphoethanolamineN-[maleimide(polyethylene glycol)-1000/2000/5000], or DSPE-PEG (1000)-maleimide/DSPE-PEG(2000)-maleimide/DSPE-PEG(5000)-maleimide (Avanti Polar Lipids, Alabaster, AL) via a thioether linkage by mixing an equimolar amount of the lipid and pure peptide in Milli-Q water (pH 7.2) at room temperature for over 24 hours with gentle agitation. The mixture was further purified by a C4 column (Phenomenex, Torrance, CA) and characterized by MALDI-TOF as described above. The fluorophore-conjugated monomer was synthesized by mixing an equimolar amount of Cyanine7 NHS ester (Lumiprobe, Hunt Vally, MD) with 1,2-distearoyl-sn-glycero-3-phosphoethanolamineN-[amino (polyethylene glycol)-1000/2000/5000] (ammonium salt) or DSPE-PEG (1000)-amine/DSPE-PEG(2000)-amine/ DSPE-PEG(5000)-amine (Avanti Polar Lipids, Alabaster, AL) in 0.1 M sodium bicarbonate solution (pH 8.3) at room temperature overnight. The mixture was purified on a C4 column and characterized byMALDI-TOF as described above.

13 types of micelles were synthesized via self-assembly. Micelles consisting of varying molar ratios of amphiphiles were synthesized: 100% DSPE-PEG-peptide, 50:50 molar ratio of DSPE-PEG-methoxy: DSPE-PEG-peptide, 90:10 molar ratio of DSPE-PEG-peptide:DSPEPEG-Cy7, and 45:45:10 molar ratio of DSPE-PEG-methoxy:DSPE-PEGpeptide: DSPE-PEG-Cy7. All monomers were dissolved in methanol or chloroform and evaporated with nitrogen to form thin films. Thin films were dried overnight under vacuum and hydrated at 80° C. for 30 min. Micelles prepared for DLS, zeta potential measurements, and TEM were hydrated in Milli-Q water, whereas micelles prepared for in vivo administration were hydrated in phosphate buffered saline (PBS).

### 2.2.4.2 Dynamic Light Scattering (DLS) and Zeta Potential Measurements

Micelles containing 100% DSPE-PEG-peptide or 50:50 by molar ratio DSPE-PEG-methoxy: DSPE-PEG-peptide were synthesized and assessed via DLS and zeta potential measurements. Micelle diameter and zeta potential were measured using Zetasizer Ultra (Malvern Instruments, Malvern, UK) at a micelle concentration of 100 μM in Milli-Q water (n=4) immediately after micelles were hydrated from thin films.

### 2.2.4.3 Transmission Electron Microscopy

Seven microliters of 100 μM micelles solution in Milli-Q water was placed onto a 200-mesh carbon TEM grid (Ted Pella, Redding, CA) for 5 min. Then, the grid was washed with Milli-Q water and negatively stained with 2 wt. % uranyl acetate solution (Polysciences, Warrington, PA). The staining solution was wicked away after 2 min, and the grid was washed with Milli-Q water again. The grid was dried overnight under room temperature in the dark and imaged on a JEOL 2100F (JEOL, Tokyo, Japan).

### 2.2.4.4 In Vivo Renal Targeting and Biodistribution

To test the renal targeting ability in vivo, 100 μl of 1,000 μM micelles containing 90:10 molar ratio of DSPE-PEG-peptide:DSPE PEG-Cy7 or 45:45:10 molar ratio of DSPE-PEG-methoxy: DSPEPEG-peptide:DSPE-PEG-Cy7 or PBS were tail vein injected with in 6-7 week old male and female C57BL/6 mice (n=4, Jackson Laboratories, Bar Harbor, ME). After 24 hours in circulation, mice were euthanized and their organs (i.e., brain, heart, lungs, liver, spleen, intestines, kidneys, and bladder) were harvested and imaged ex vivo via Ami HTX (Spectral Instruments Imaging, Tucson, AZ). Qualification of the fluorescence signal was conducted to determine the biodistribution of particles by Aura imaging software (Spectral Instruments Imaging, Tucson, AZ). All animal experiments were approved by University of Southern California (USC) Institutional Animal Care and Use Committee (IACUC).

### 2.2.4.5 Histology

Immediately after ex vivo imaging, harvested organs were flash frozen in 2-methylbutane and liquid nitrogen, embedded in optimum cutting temperature (OCT) compound, and sectioned into 8 μm samples via a CM3050 S Cryostat (Leica CM3050S, Leica, Wetzlar Germany). Tissue sections were then stained with hematoxylin and eosin (H&E) and imaged with a microscope (Leica DMi8, Leica, Wetzlar, Germany).

### 2.2.4.6 Immunohistochemistry

To assess micelle colocalization with megalin, kidney tissue sections were first washed with Tris buffered saline (TBS) plus 0.025% Triton X-100 with gentle agitation for 10 minutes. A block buffer with 1% bovine serum albumin (BSA), 10% normal goat serum, and 0.3 m glycine in 0.1% PBS Tween was applied to the slides for 1 hour at room temperature. Then, the slides were applied with an anti-Lrp2/Megalin antibody (Abcam, Cambridge, UK, 1:100) overnight at 4° C. The following day, slides were rinsed twice with TBS plus 0.025% Triton for 5 minutes and applied with fluorophore-conjugated secondary antibody-goat, antimouse IgG H&L Alexa Fluor® 488 (Abcam, Cambridge, UK, 1:1000) for 1 hour at room temperature in the dark. Slides were then counterstained with DAPI and mounted with VectaMount™ mounting medium (Vector Laboratories, Burlingame, CA). Fixed slides were imaged with a LSM 700 confocal microscope (Zeiss, Oberkochen, Germany), and the colocalization between Cy7 and Alexa Fluor® 488 channels was performed on ImageJ with coloc2.

TABLE 2.2
BUN and urine creatinine levels upon micelle

| NP | BUN (mg/dl) | Creatinine (mg/dl) |
|---|---|---|
| 45% PEG2000-(KKEEE)₃K | 40.8 ± 12.6 | 5.5 ± 1.8 |
| 45% PEG2000-(KKEEE)₂K | 64.6 ± 33.3 | 5.7 ± 3.1 |
| 45% PEG2000-(KKEEE)K | 47.9 ± 13.9 | 4.2 ± 0.2 |
| 45% PEG2000-(EEKKK)₃E | 35.2 ± 8.6 | 5.5 ± 1.9 |
| 45% PEG2000-(EEKKK)₂E | 60.7 ± 30.4 | 9.0 ± 3.5 |
| 45% PEG2000-(EEKKK)E | 48.4 ± 12.8 | 4.5 ± 2.6 |
| 45% PEG2000-KKKKK | 41.4 ± 1.6 | 4.9 ± 0.4 |
| 45% PEG2000-EEEEE | 48.7 ± 4.3 | 5.6 ± 3.4 |
| 90% PEG2000-(KKEEE)₃K | 29.5 ± 6.8 | 4.1 ± 1.7 |
| 90% PEG2000-(EEKKK)₃E | 26.3 ± 16.0 | 3.9 ± 1.2 |
| 45% PEG1000-(KKEEE)₃K | 39.5 ± 4.1 | 5.0 ± 3.1 |
| 90% PEG5000-(KKEEE)₃K | 54.0 ± 10.9 | 5.1 ± 1.0 |
| 90% PEG2000-NT | 36.2 ± 5.5 | 4.4 ± 3.9 |
| PBS | 47.1 ± 9.4 | 7.6 ± 5.4 |

### 2.2.4.7 Kidney Health

Renal health was assessed by analyzing blood urea nitrogen and urine creatinine levels in serum and urine. BUN was analyzed by a BUN enzymatic kit (Bioo scientific, Austin, TX), and urine creatinine was assessed by a mouse creatinine enzymatic kit (Crystal Chem, Elk Grove Village, IL)

### 2.2.4.8 Statistical Analysis

All statistical analyses were performed using GraphPad Prism 8 (San Diego, CA). Analysis of variance (ANOVA) with a Tukey's test for post-hoc analysis was used to determine statistical significance and p≤0.05 was considered to be significant.

### 3. Micelle Nanoparticles Loaded in Oral, Chitosan Nanoparticles

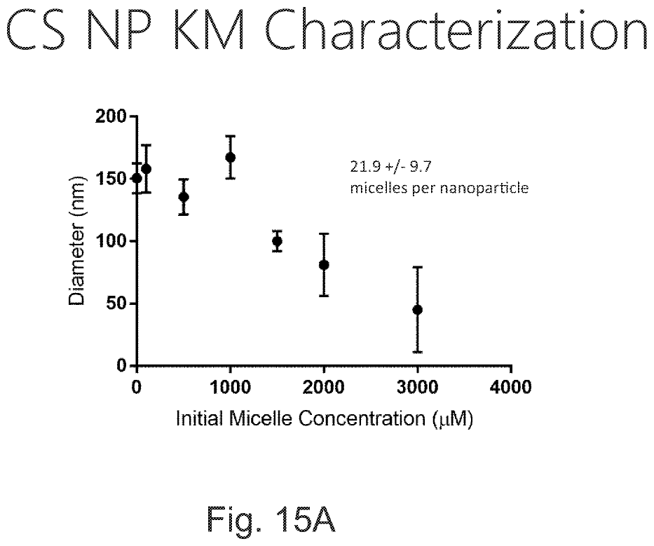

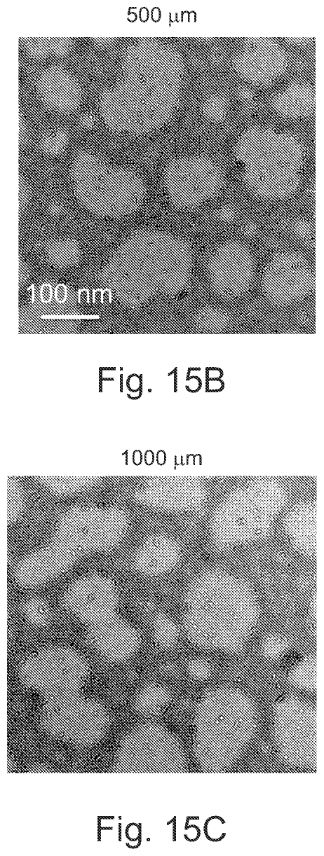

FIGS. 15A, 15B, and 15C provide characterization of KM micelles loaded within CS NP. In FIG. 15A, DLS measurements show that the unloaded CS-NP diameter of 150 nm is preserved when up to 1000 μM of micelles are loaded within. FIGS. 15B and 15C provide TEM images showing spherical CS-NP KM of approximately 150 nm, with micelles dispersed throughout. Image analysis indicates 21.9+/−9.7 micelles per CS-NP.

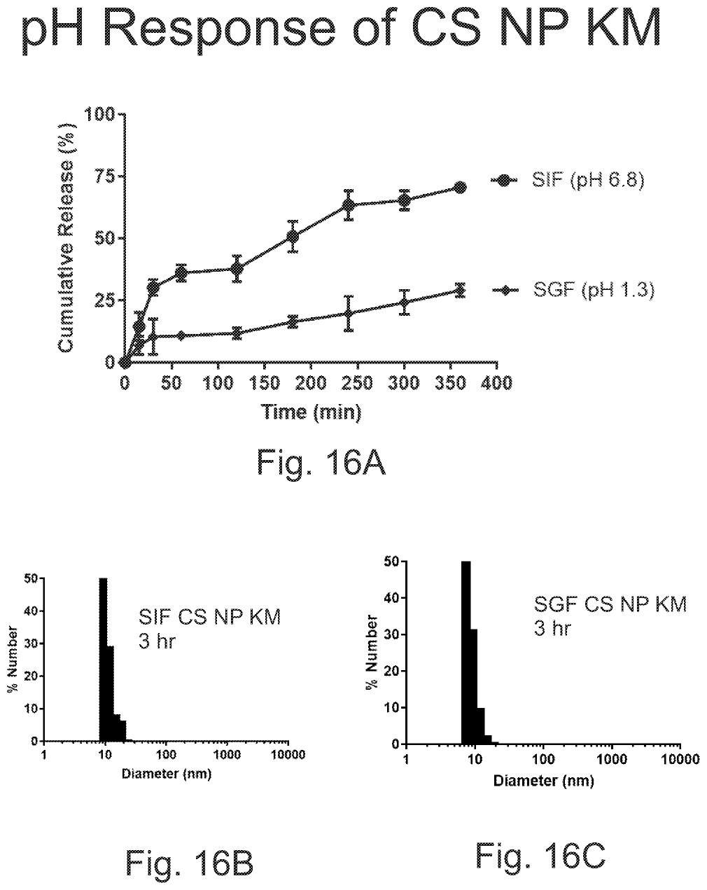

FIGS. 16A, 16B, and 16C provide experimental results showing favorable pH responsive release of KM from CS-NP. With respect to FIG. 16A, in vitro release of FITC labeled KM from CS-NP conditions present in the GI tract (pH=1.3 simulated gastric fluid (SGF); 6.8 simulated intestinal fluid (SIF)) (N≥4). Release is minimal (<25%) at low pH, but increases at pH conditions in the intestines, which suggests CS-NP payload will be protected in the stomach and released in the intestines. In FIG. 16B/C, DLS of supernatant at the final timepoint confirms intact micelle present in solution.

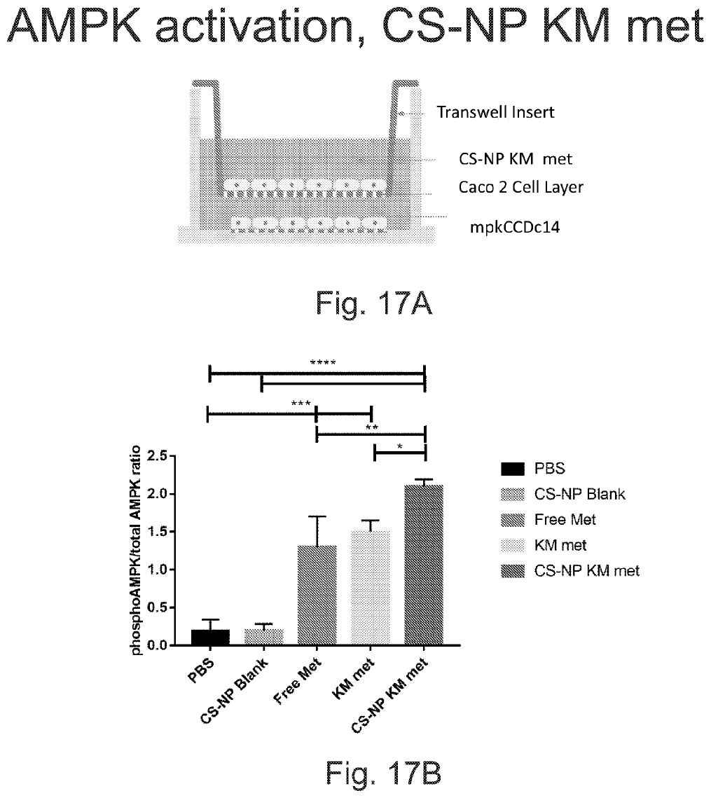

FIGS. 17A and 17B demonstrate that CS-NP met is able to bypass an in vitro Transwell intestinal cell model and deliver a therapeutic, metformin, to cells in the basolateral chamber. FIG. 17A provides a transwell schematic of mpkCCDc14 cell monolayers, with a Caco2 cell monolayer seeded on the transwell insert. Referring to FIG. 17B, phosphorylated AMPK to total AMPK obtained via ELISA of mpkCCDc14 treated for up to 48 h with CS-NP met (300 μM), free met, KM met, CS NP Blank, and PBS show a significant increase for the CS-NP met, KM met, and free met groups, compared to CS-NP, confirming therapeutic activity (****p≤0.0001, ***p≤0.001, N≥4).FIG. 13A. Chitosan Disrupts Intestinal Tight Junctions.

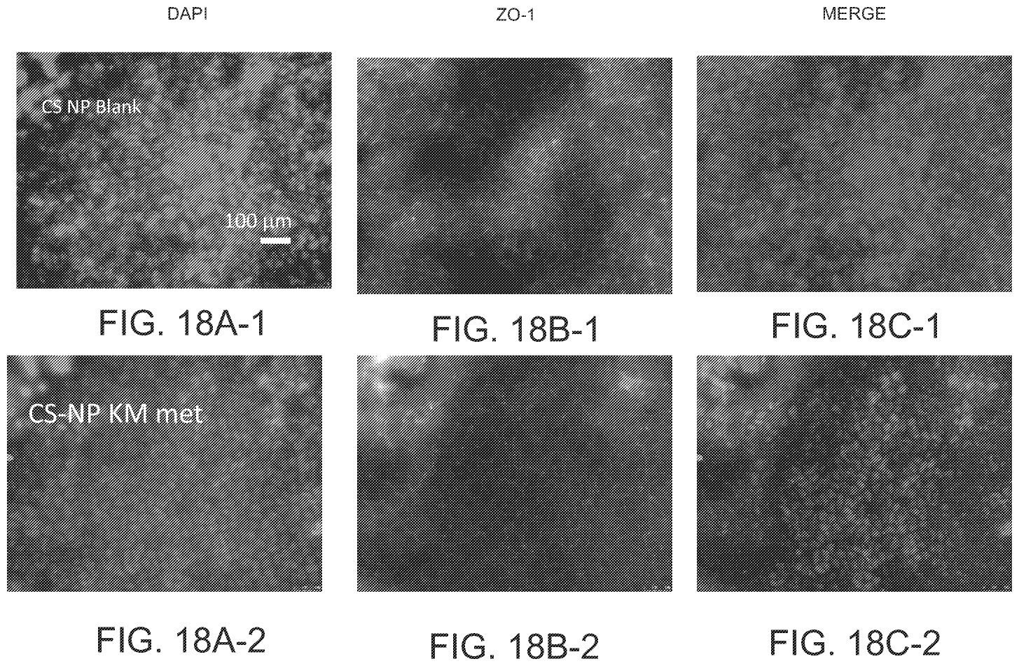

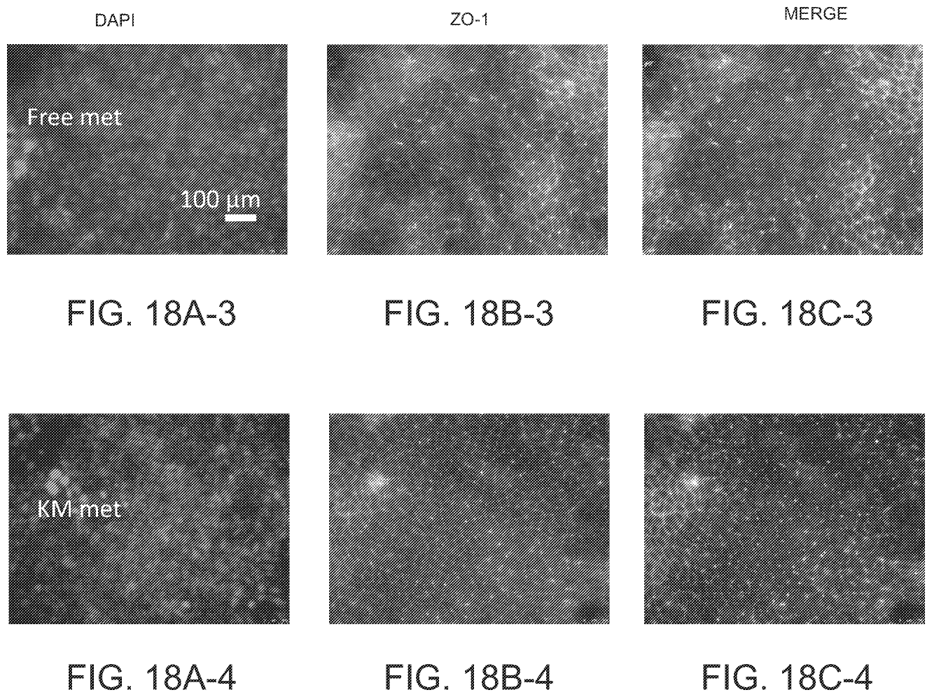

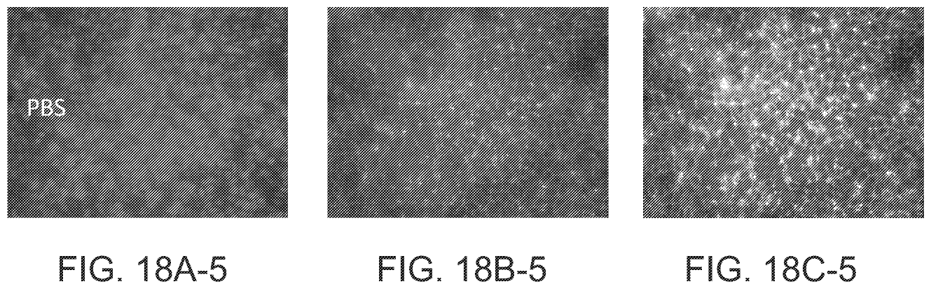

FIG. 18 shows that CS-NP disrupts tight junctions in Caco-2 cell monolayers. Caco-2 cell monolayers were incubated with CS-NP met, free met, KM met, CS NP Blank, and PBS for 3 hours. Barrier tight junction integrity was stained with ZO-1, and cell nuclei with DAPI. Barrier disruption is only observed in groups containing chitosan.

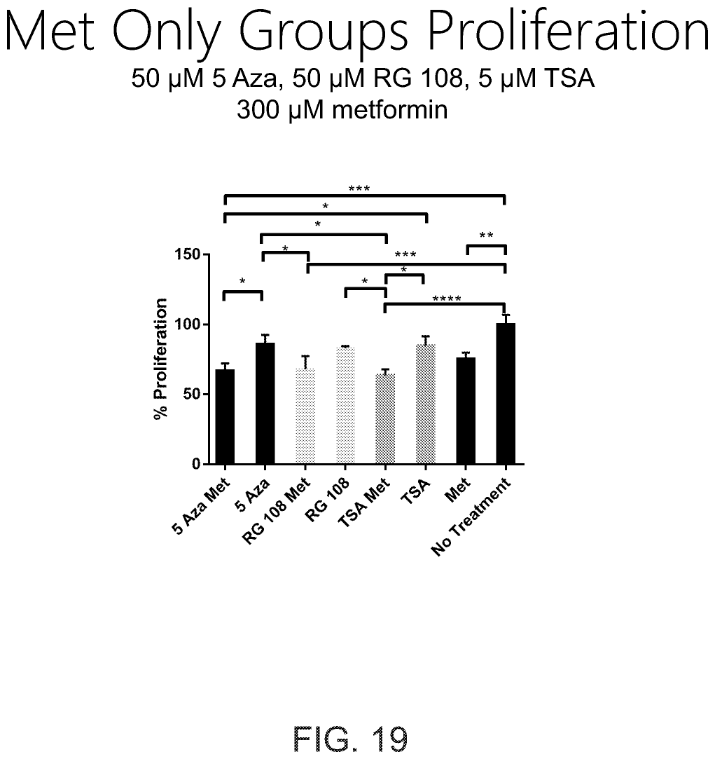

Referring to FIG. 19, biocompatibility was assessed on PKD1 heterozygous cells with an MTS assay, and demonstrates decrease in proliferation when an ADPKD drug and an epigenetic modifier is combined. Cells were incubated with either free 50 μM 5 Aza, 50 μM RG 108, or 5 μM TSA (dissolved DMSO, and added to complete media at 1% final vol/vol), as well as 300 μM metformin, or 300 μM metformin in micelle formulations for 24 hours, on a 96-well plate before the addition of MTS reagent.

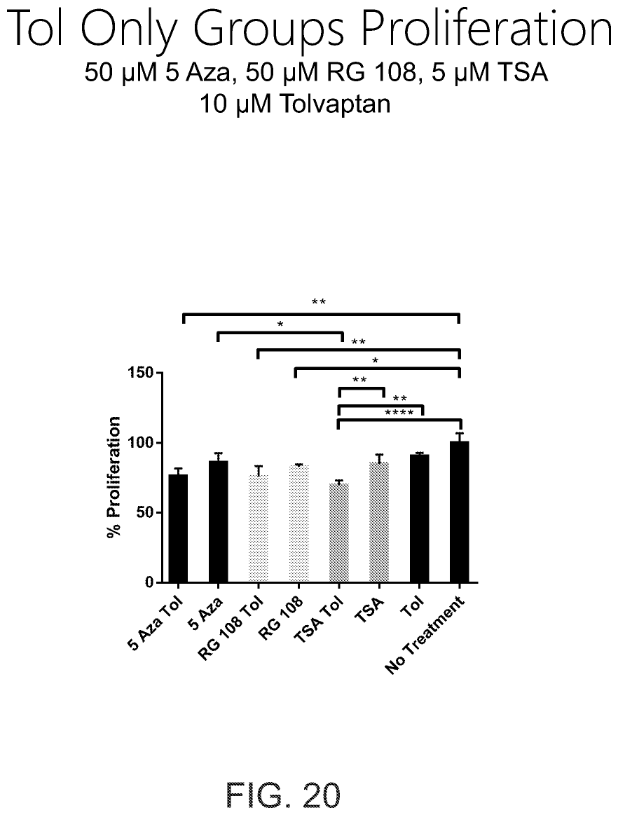

Referring to FIG. 20, biocompatibility was assessed on PKD1 heterozygous cells with an MTS assay. A decrease in proliferation was demonstrated when an ADPKD drug and an epigenetic modifier is combined. Cells were incubated with either free 50 μM 5 Aza, 50 μM RG 108, or 5 μM TSA (dissolved DMSO, and added to complete media at 1% final vol/vol), as well as 10 μM Tolvaptan, or 10 μM Tolvaptan in micelle formulations for 24 hours, on a 96-well plate before the addition of MTS reagent.

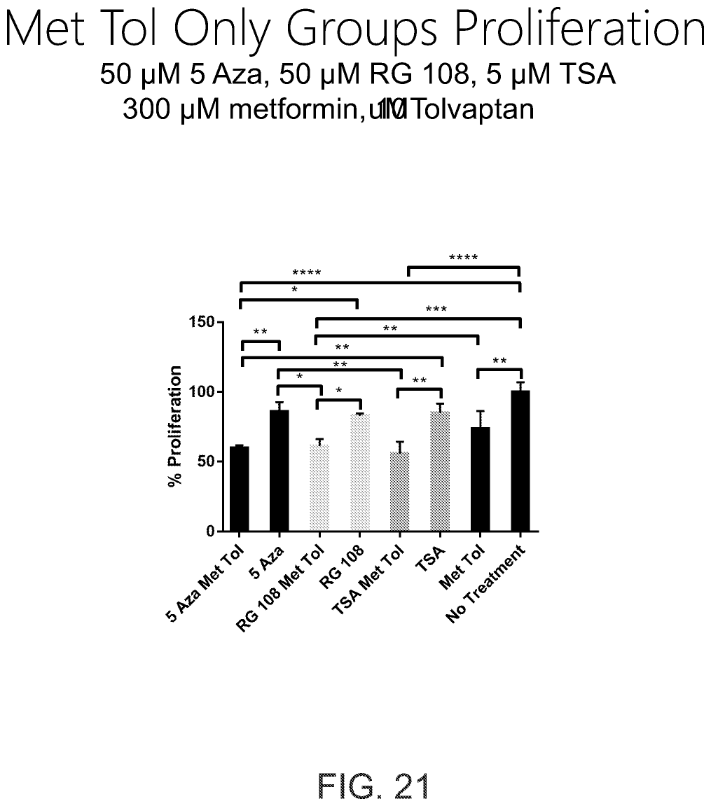

Referring to FIG. 21, biocompatibility was assessed on PKD1 heterozygous cells with an MTS assay. A decrease in proliferation was demonstrated when an ADPKD drug and an epigenetic modifier is combined. PKD1 Het were incubated with either free 50 μM 5 Aza, 50 μM RG 108, or 5 μM TSA (dissolved DMSO, and added to complete media at 1% final vol/vol), as well as either 300 μM metformin, 10 μM Tolvaptan, or both 300 μM metformin with 10 μM Tolvaptan in micelle formulations for 24 hours, on a 96-well plate before the addition of MTS reagent.

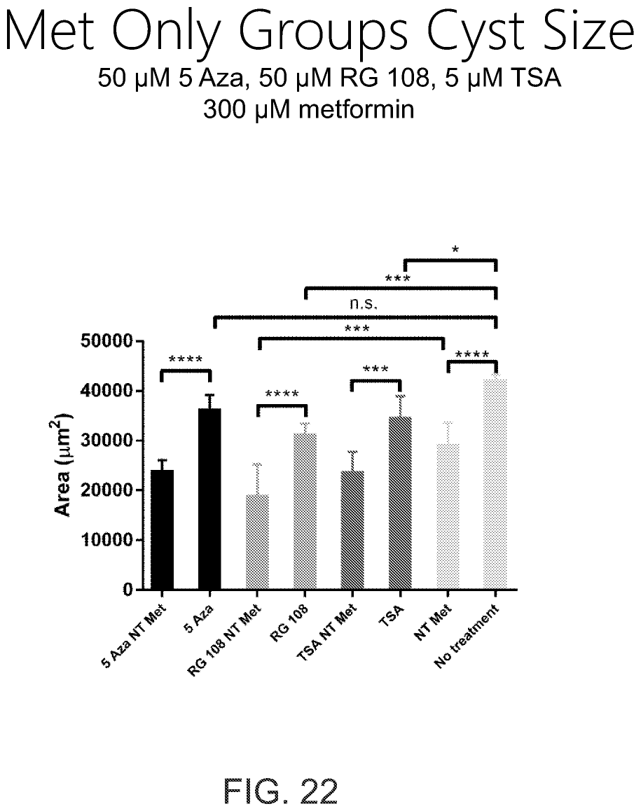

FIG. 22 provides a bar graph showing that cyst area in PKD1 heterozygous cells is reduced when an ADPKD drug and an epigenetic modifier is combined. Cells seeded in 2% Matrigel were incubated with either free 50 μM 5 Aza, 50 μM RG 108, or 5 μM TSA (dissolved DMSO, and added to complete media at 1% final vol/vol), as well as 300 μM metformin, or 300 μM metformin in micelle formulations for up to 10 days hours, on a 96-well plate.

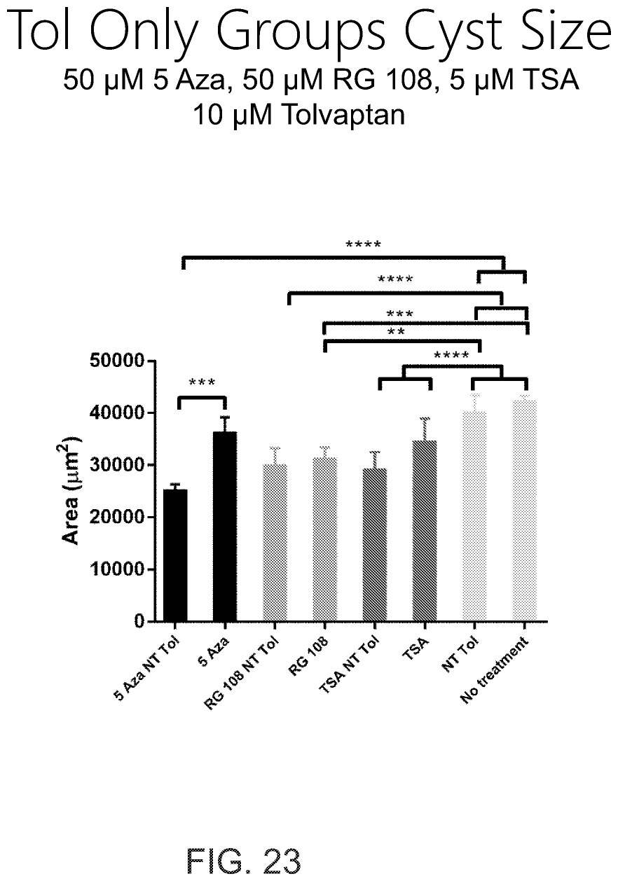

FIG. 23 provides a bar chart showing that cyst area in PKD1 het cells is reduced when an ADPKD drug and an epigenetic modifier is combined. Cells seeded in 2% Matrigel were incubated with either free 50 μM 5 Aza, 50 μM RG 108, or 5 μM TSA (dissolved DMSO, and added to complete media at 1% final vol/vol), as well as 10 μM Tolvaptan, or 10 μM Tolvaptan in micelle formulations for up to 10 days hours, on a 96-well plate.

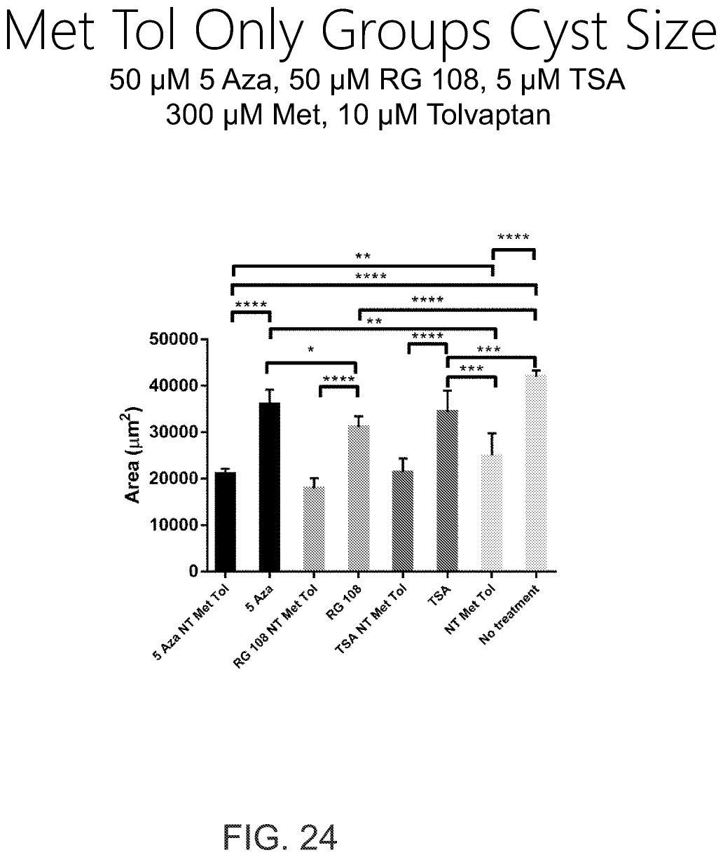

FIG. 24 provides a bar graph showing that cyst area in PKD1 heterozygous cells is reduced when multiple ADPKD drugs and an epigenetic modifier is combined. Cells seeded in 2% Matrigel were incubated with either free 50 μM 5 Aza, 50 μM RG 108, or 5 μM TSA (dissolved DMSO, and added to complete media at 1% final vol/vol), as well as either 300 μM metformin, 10 μM Tolvaptan, or both 300 μM metformin with 10 μM Tolvaptan in micelle formulations for up to 10 days hours, on a 96-well plate.

4. Additional therapeutic payloads.
4.1 Micelle Formation with Everolimus or Rapamycin Payloads

Everolimus and rapamycin, hydrophobic small molecule drugs that act as mTOR activation inhibitors were loaded into micelles for drug delivery for autosomal dominant polycystic kidney disease (ADPKD) therapy.

Micelles were assembled by dissolving DSPE-PEG(2000)-methoxy in methanol at 100 μM with either everolimus or rapamycin at different lipid:drug molar ratios (2:1, 5:1, 10:1, 20:1), mixing the components, and evaporating the organic solvent under nitrogen. The resulting film was dried under vacuum over night, and then hydrated at 80° C. for 30 min in water and allowed to cool to room temperature overnight before DLS and TEM analysis. DLS measurements were determined at 90° and 637 nm using a Malvern Zetasizer. Free drug was separated by syringe filtering micelles using PTFE syringe filter, pore size 0.1 μm. To calculate encapsulation efficiency and drug loading, micelles were disrupted using DMSO and encapsulated drug was quantified by reading absorbance at 278 nm in a UV Greiner 96-well plate.

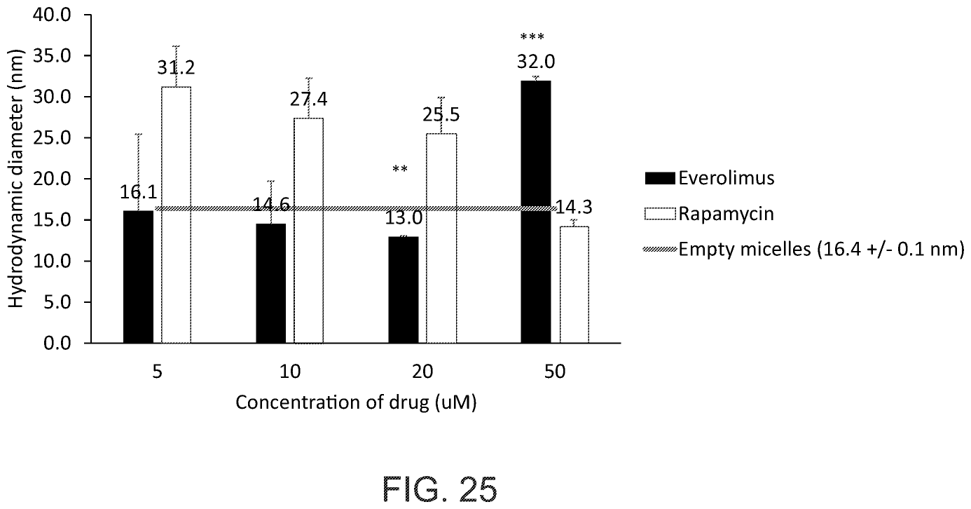

FIG. 25 provides a bar graph of the hydrodynamic diameter of micelles containing everolimus or rapamycin at different concentrations, n=3. The solid line indicates the size of empty micelles. The size of drug-containing micelles was compared to that of empty micelles using Student's t test (two-tailed), **p<0.01, ***p>0.001.

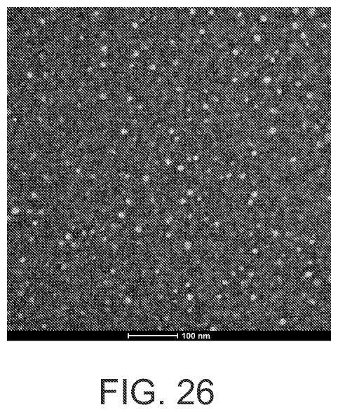

FIG. 26 provide transmission electron microscopy (TEM) images of representative micelles encapsulated with 10 μM everolimus or 10 μM rapamycin. All micelle concentrations are at 100 μM. The TEM images confirm the DLS results of FIG. 25 and indicate the presence of spherical, small, monodisperse micelles when encapsulated with these drugs.

Table 4.2 provides a summary of encapsulation study results for the lipid:drug ratio 2:1, which showed the highest encapsulation efficiency. **p=0.004, Students t test comparing size to blank micelles (6.4±0.3 nm). These results show that micelles can efficiently encapsulate rapamycin and everolimus for drug delivery.

TABLE 4.2
Encapsulation study results.

| | | | | | | Moles | |
|---|---|---|---|---|---|---|---|
| | | | | DSPE- | | drug | |
| | Solubility | Molar | | PEG(2000)- | Loading | per | |
| | in MQ | Mass | Encapsulation | methoxy:drug | capacity | mole | Size |
| | (mg/ml) | (g/mol) | efficiency (%) | molar ratio | (wt/wt %) | micelle | (nm) |
| Everolimus | <0.1 | 958.224 | 87.1 ± 2.0 | 2:1 | 13.0 ± 0.3 | 0.1 ± 0.01 | 21.0 ± 1.3 ** |
| Rapamycin | 0.005-0.02 | 914.172 | 88.6 ± 5.9 | 2:1 | 12.6 ± 0.7 | 0.1 ± 0.001 | 9.4 ± 0.4 |

where:

$$Encapulation efficiency = \frac{Weight of drug entrapped within nanoparticle}{Total Drug added} \times 100 \%$$

$$Encapulation Capacity = \frac{Weight of drug entrapped within nanoparticle}{Total Weight of nanoparticles} \times 100 \%$$

### 4.2 Novel Targeting Peptides MGSHIEPGG (SEQ ID NO: 15) and KMGGTNHPE (SEQ ID NO: 16)

Table 4.3 provides a summary of cortical collecting duct (CCD)-targeting peptides identified in the literature. These peptides could be incorporated in micelles to promote targeted delivery to the CCD, where cyst formation begins in ADPKD. PCT: proximal convoluted tubule, MW: molecular weight.

TABLE 4.3
Summary of cortical collecting duct (CCD)-targeting
peptides identified in the literature

| Targeting | Targeting | | Water | |
|---|---|---|---|---|
| peptide | location | Identified by | solubility | MW |
| MGSHIEPGG-C | PCT and CCD | Ex vivo phage | Good | 1029 |
| (AC1) (SEQ | | display (rat) | | |
| ID NO: 52) | | | | |
| KMGGTNHPE-C | PCT and CCD | Ex vivo phage | Good | 1115 |
| (AC2) (SEQ | to a lesser | display (rat) | | |
| ID NO: 19) | extent | | | |

### 4.2.1 Peptide Synthesis MGSHIEPGGC (SEQ ID NO: 52) and KMGGTNHPEC (SEQ ID NO: 53)

In these experiments the peptides MGSHIEPGG-C(SEQ ID NO: 52) and KMGGTNHPE-C(SEQ ID NO: 53) were synthesized. A cysteine was added the end of each sequence to facilitate conjugation to malemide on the hydrophobic phospholipid. The peptides were synthesized using standard Fmoc-mediated solid phase peptide synthesis methods on rink amide resin (Anaspec, Fremont, CA) using an automated PS3 Benchtop Peptide Synthesizer (Protein Technologies, Tucson, AZ). The N-terminus was acetylated using 10× molar excess of acetic anhydride in DMF. Peptides were cleaved and deprotected with 94:2.5:2.5:1 by volume trifluoroacetic acid: 1,2-ethanedithiol: H₂O:triisopropylsilane and were precipitated and washed several times with cold diethyl ether, dissolved in water, lyophilized, and stored as lyophilized powders at −20° C. Crude peptide mixtures were purified by reverse-phase HPLC (Prominence, Shimadzu, Columbia, MD, USA) on a C8 column (Waters, Milford, MA, USA) at 55° C. using 0.1% trifluoroacetic acid in acetonitrile/water mixtures and characterized by MALDI-TOF mass spectral analysis (Biflex III, Bruker, Billerica, MA, USA).

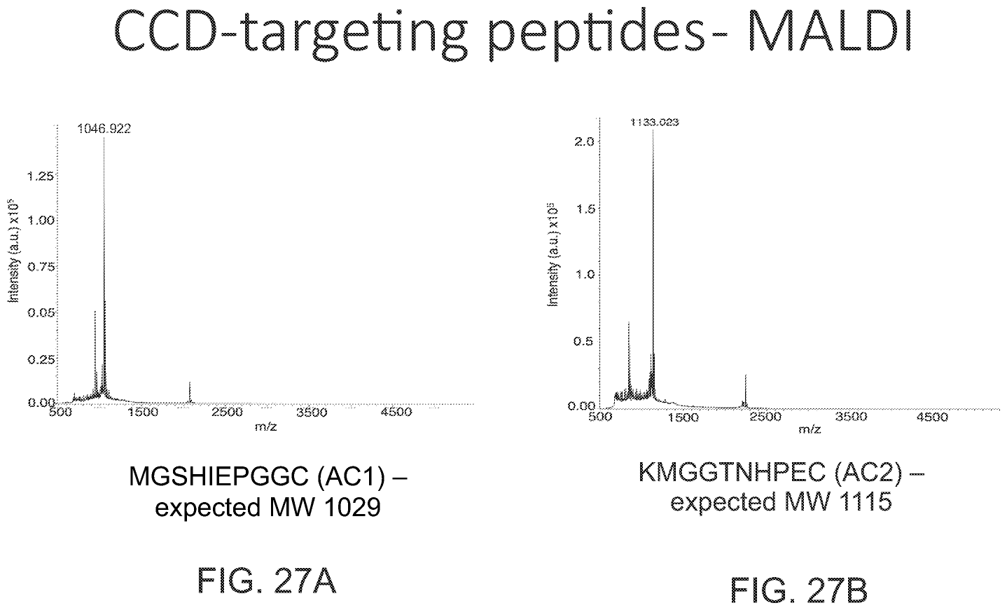

FIGS. 27A and 27B provide MALDI-TOF spectra indicating the molecular weight of CCD-targeting peptides MGSHIEPGG-C(SEQ ID NO: 52) and KMGGTNHPE-C(SEQ ID NO: 53). The spectra indicated that peptides were successfully synthesized. Advantageously, these peptides can be incorporated in micelles to promote targeted delivery to the CCD, where cyst formation begins in ADPKD.

### 4.2.2 Conjugation of MGSHIEPGGC (SEQ ID NO: 52) and KMGGTNHPEC (SEQ ID NO: 53) to Lipid for PAM Formation

Cysteine-containing peptides were conjugated via a thioether linkage to 1,2-distearoyl-sn-glycero-3-phosphoethanolamine-N— [maleimide(polyethylene glycol)-2000], or DSPE-PEG(2000)-maleimide (Avanti Polar Lipids, Alabaster, AL, USA) by adding peptide to lipid at a 1:1 molar ratio in water. After reaction at room temperature shaking for 24 h, the resulting product was purified on a C4 column and characterized by MALDI-TOF.

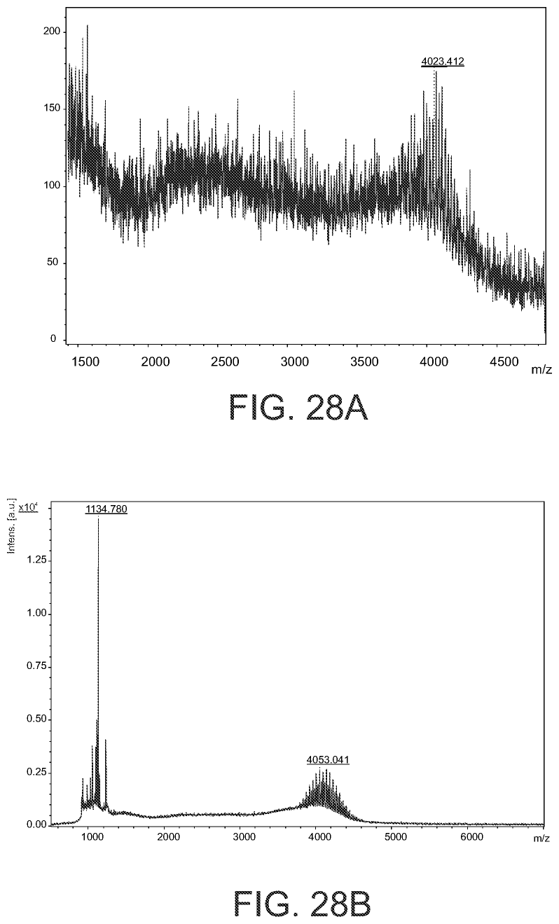

FIGS. 28A and 28B provide MALDI-TOF spectra indicating the successful conjugation of CCD-targeting peptides to DSPE-PEG(2000)-maleimide. Advantageously, these conjugates can be used to create micelles that actively target the CCD, where cyst formation begins in ADPKD.

4.3.3 Micelle Formation with MGSHIEPGG-C(SEQ ID NO: 52) and KMGGTNHPE-C(SEQ ID NO: 53) amphiphiles

Micelles were assembled by dissolving the peptide-containing DSPE-PEG(2000) amphiphiles in methanol at 100 μM, mixing the components, and evaporating the organic solvent under nitrogen. The resulting film was dried under vacuum over night, and then hydrated at 80° C. for 30 min in water and allowed to cool to room temperature overnight before DLS or TEM analysis. DLS measurements were determined at 90° and 637 nm using a Malvern zetasizer. Negatively stained samples for TEM were prepared by layering the PAMs on 400 mesh lacey carbon grids (Ted Pella, Redding, CA) for 2 min. Excess liquid was wicked away with filter paper before placing 1 wt. % uranyl acetate solution for 2 min. Excess liquid was wicked away with filter paper and samples were dried overnight before imaging on a JEOL 1230 TEM (JEOL, Ltd., Tokyo, Japan).

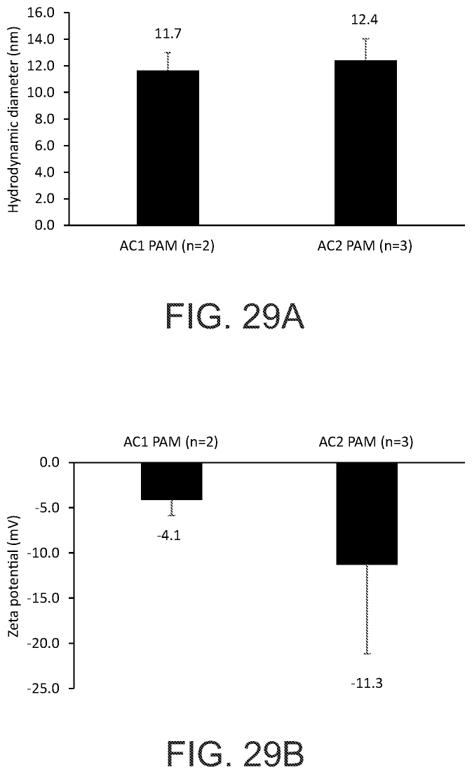

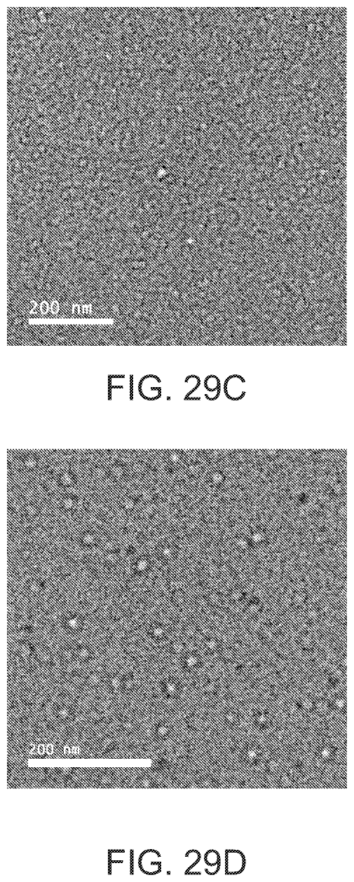

FIGS. 29A, 29B, 29C, and 29D provide experimental results for the characterization of CCD-targeting micelles. Referring to FIG. 29A, hydrodynamic diameter of micelles as measured by DLS. FIG. 29B provide Zeta potential of PAMs. FIGS. 29C and 29D provide TEM images of PAMs. These results confirm that CCD-targeting micelles are small and slightly negatively charged.

### 4.3.4 Alternative Cortical Collecting Duct-Targeting Peptides

Table 4.4 provides a summary of alternative cortical collecting duct (CCD)-targeting peptides identified in the literature. These peptides can be incorporated in micelles to promote targeted delivery to the CCD, where cyst formation begins in ADPKD. MW: molecular weight.

TABLE 4.4

| | Targeting | | Water | |
|---|---|---|---|---|
| Targeting peptide | location | Identified by | solubility | MW |
| CKDSPKSSKSIRFIPVST | Type A | From C- | Good | 2021.34 |
| (CKD) | intercalated | terminal mouse | | |
| (SEQ ID NO: 19) | cells of | V1aR portion | | |
| | collecting duct | | | |
| ELRGDRAKL-C | CCD | Ex vivo phage | Good | 1229.42 |
| (RGD) | | display (rat) | | |
| (SEQ ID NO: 56) | | | | |

### 5. Targeting Peptide GRGDSPC

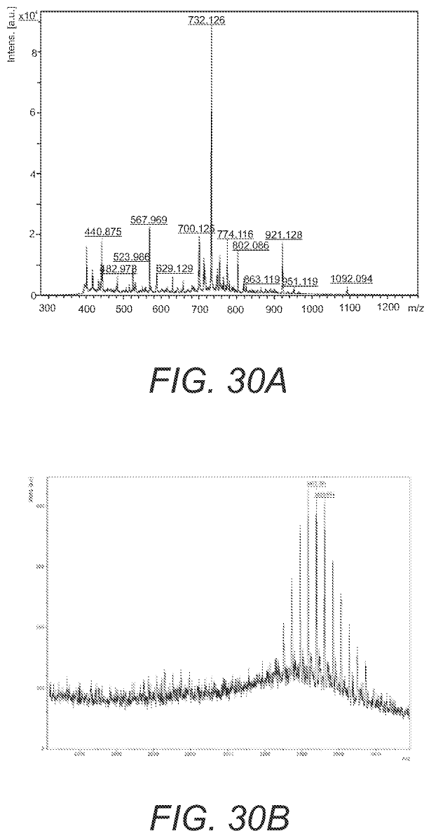

FIGS. 30A and 30B provides A) MALDI characterization of GRGDSPC (expected m/z: 731 g/mol). B) MALDI characterization of DSPE-PEG(2000)-GRGDSP (expected m/z: 3531 g/mol).

### 5.1 Synthesis of Targeting Peptide GRGDSPC

Peptides were synthesized using standard Fmoc-mediated solid phase peptide synthesis on an automatic PS3 peptide synthesizer (Protein Technologies, Tucson, AZ, USA) with rink Amide resin (Protein Technologies, Tucson, AZ, USA). A cysteine was added to the N-terminus of all the peptide sequences in order to make a thioether linkage reaction. The peptides were then cleaved from the resin with 94:2.5:2.5:1 volume ratios of trifluoroacetic acid: 1,2-ethanedithiol: H₂O:triisopropylsilane. Cleaved peptides were precipitated and washed several times with ice cold diethyl ether, dissolved in Milli-Q water, lyophilized, and stored at −20° C. The crude peptides were purified by reverse-phase high performance liquid chromatography (HPLC) (Shimadzu, Kyoto, Japan) on a C18 column (Phenomenex, Torrance, CA, USA) at 55° C. with 0.1% formic acid in acetonitrile/water mixture. The purified peptides were characterized using matrix-assisted laser desorption ionization time-of-flight mass spectral analysis (MALDI-TOF) (Autoflex speed, Bruker, Billerica, MA, USA) and conjugated to 1,2 distearoyl-sn-glycero-3-phosphoethanolamineN-[maleimide(polyethylene glycol)-2000], or DSPE-PEG(2000)-maleimide (Avanti Polar Lipids, Alabaster, AL, USA) via a thioether linkage by mixing an equimolar amount of the lipid and pure peptide in Milli-Q water (pH 7.2) at room temperature for over 24 hours with gentle agitation. The mixture was further purified by a C4 column (Phenomenex, Torrance, CA, USA) and characterized by MALDI-TOF as described above.

DSPE-PEG(2000)-GRGDSP monomers were dissolved in methanol or chloroform and evaporated with nitrogen to form thin films. Thin films were dried overnight under vacuum and hydrated at 80° C. for 30 min. Micelles prepared for DLS and zeta potential measurements were hydrated in Milli-Q water

Micelles containing 100% DSPE-PEG(2000)-GRGDSP were synthesized and assessed via DLS and zeta potential measurements. Micelle diameter and zeta potential was measured using Zetasizer Ultra (Malvern Instruments, Malvern, UK) at a micelle concentration of 100 μM in Milli-Q water (n=4) immediately after micelles were hydrated from thin films.

5.1 Micelles Loaded with Various Payloads

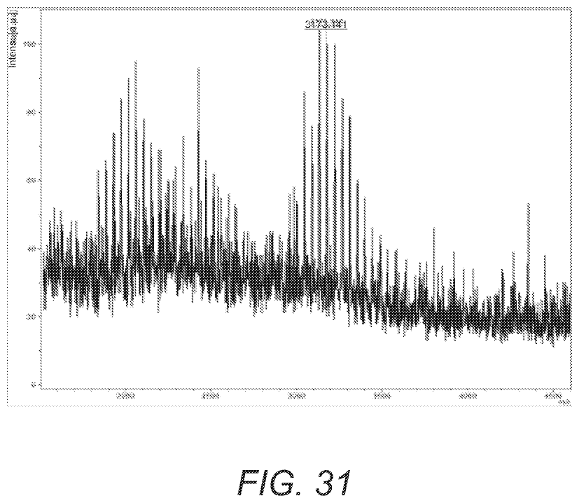

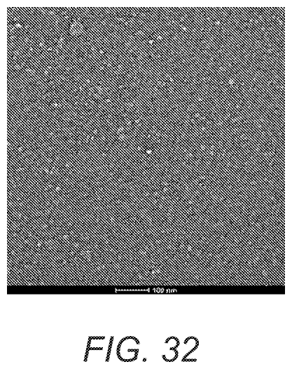

FIG. 31 provides MALDI characterization of DSPE-PEG(2000)-pravastatin (expected m/z: 3200 g/mol). FIG. 32 provides TEM image of DSPE-PEG(2000)-pravastatin micelles.

FIG. 32 provide a TEM image of pravastatin-loaded micelles.

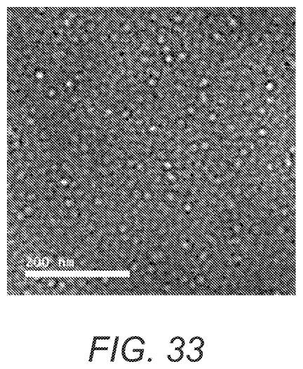

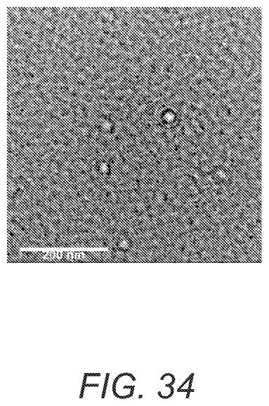

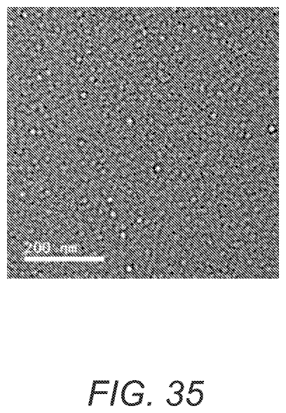

FIG. 33 provide a TEM image of octreotide-loaded micelles.

FIG. 36 provides a TEM image of bardoxolone methyl-loaded micelles.

FIG. 38 provide a TEM image of salsalate-loaded micelles.

4.3.5 mRNA Payload

Synthesis and characterization of micelles loaded with mRNA. Micelles will be incubated and adsorbed with mRNA (e.g., PKD2 transcript Accession NM_000297.4) in PBS at RT for 1 h. Free mRNA will be removed from solution using ultrafiltration and DLS and TEM will be used to evaluate size, ζ potential and polydispersity of the resulting mRNA-micelles. Adsorbed mRNA will be quantified using RT-PCR (Qiagen). Multiple weight ratios of mRNA:micelles (1:10, 1:20, 1:30) will be used to determine the maximum amount of mRNA that can be bound. Stability of mRNA-micelles from 1-30 days will be evaluated by measuring size, polydispersity, and ζ potential with DLS.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

[1] C. L. Ventola, Progress in Nanomedicine: approved and investigational nanodrugs, P & T: Peer-rev. J. Formul. Manag. 42 (12) (2017) 742-755.
[2] S. M. Rafiyath, et al., Comparison of safety and toxicity of liposomal doxorubicin vs. conventional anthracyclines: a meta-analysis, Exp. Hematol. Oncol. 1 (1) (2012) 10.
[3] T. M. Allen, P. R. Cullis, Drug delivery systems: entering the mainstream, Science 303 (5665) (2004) 1818.
[4] D. D. Chin, et al., Collagenase-cleavable peptide amphiphile micelles as a novel theranostic strategy in atherosclerosis, Adv. Therapeutics (2020) 1900196, n/a (n/a).
[5] D. D. Chin, et al., Hydroxyapatite-binding micelles for the detection of vascular calcification in atherosclerosis, J. Mater. Chem. B 7 (41) (2019) 6449-6457.
[6] C. Zavaleta, D. Ho, E. J. Chung, Theranostic nanoparticles for tracking and monitoring disease state, SLAS Technol.: Transl. Life Sci. Innov. 23 (3) (2017) 281-293.
[7] S. Shen, et al., Folate-conjugated nanobubbles selectively target and kill cancer cells via ultrasound-triggered intracellular explosion, Biomaterials 181 (2018) 293-306.
[8] F. A. U. Rahman, S. Ali, M. W. Saif, Update on the role of nanoliposomal irinotecan in the treatment of metastatic pancreatic cancer, Ther. Adv. Gastroenterol. 10 (7) (2017) 563-572.
[9] A. Kolb-Maurer, et al., An update on peginterferon beta-la management in multiple sclerosis: results from an interdisciplinary Board of German and Austrian Neurologists and Dermatologists, BMC Neurol. 19 (1) (2019) 130.
[10] P. Curley, N. J. Liptrott, A. Owen, Advances in nanomedicine drug delivery applications for HIV therapy, Future Sci. OA 4 (1) (2017) FSO230.
[11] M. Levy, J. Feingold, Estimating prevalence in single-gene kidney diseases progressing to renal failure, Kidney Int. 58 (3) (2000) 925-943.
[12] J. Wang, J. J. Masehi-Lano, E. J. Chung, Peptide and antibody ligands for renal targeting: nanomedicine strategies for kidney disease, Biomater. Sci. 5 (8) (2017) 1450-1459.
[13] J. Wang, et al., Design and in vivo characterization of kidney-targeting multimodal micelles for renal drug delivery, Nano Res. 11 (10) (2018) 5584-5595.
[14] A. Masoumi, et al., Developments in the management of autosomal dominant polycystic kidney disease, Ther. Clin. Risk Manag. 4 (2) (2008) 393-407.
[15] A. B. Chapman, et al., Autosomal dominant polycystic kidney disease (ADPKD): executive summary from a kidney disease: improving global outcomes (KDIGO) controversies conference, Kidney Int. 88 (1) (2015) 17-27.
[16] R.-U. Müller, T. Benzing, Management of autosomal-dominant polycystic kidney disease-State-of-the-art, Clin. Kidney J. 11 (Suppl_1) (2018) i2-i13.
[17] F. A. Belibi, C. L. Edelstein, Novel targets for the treatment of autosomal dominant polycystic kidney disease, Expert Opin. Investig. Drugs 19 (3) (2010) 315-328.
[18] M. V. Irazabal, V. E. Torres, Experimental therapies and ongoing clinical trials to slow down progression of ADPKD, Curr. Hypertens. Rev. 9 (1) (2013) 44-59.
[19] E. J. Chung, K. R. Hallows, “First do no harm”: kidney drug targeting to avoid toxicity in ADPKD, Am. J. Physiol.-Renal Physiol. 315 (3) (2018) F535-F536.
[20] V. Takiar, et al., Activating AMP-activated protein kinase (AMPK) slows renal cystogenesis, Proc. Natl. Acad. Sci. 108 (6) (2011) 2462.
[21] L. J. McCreight, C. J. Bailey, E. R. Pearson, Metformin and the gastrointestinal tract, Diabetologia 59 (2016) 426-435.
[22] K. Kanto, et al., Effects of dosage and dosing frequency on the efficacy and safety of high-dose metformin in Japanese patients with type 2 diabetes mellitus, J. Diab. Invest. 9 (3) (2017) 587-593.
[23] G. G. Graham, et al., Clinical pharmacokinetics of metformin, Clin. Pharmacokinet. 50 (2) (2011) 81-98.
[24] P. McEwan, et al., A model to predict disease progression in patients with autosomal dominant polycystic kidney disease (ADPKD): the ADPKD outcomes model, BMC Nephrol. 19 (1) (2018) 37.
[25] S. Patra, et al., Short term efficacy and safety of low dose tolvaptan in patients with acute decompensated heart failure with hyponatremia: a prospective observational pilot study from a single center in South India, Heart Views: Off. J. Gulf Heart Assoc. 15 (1) (2014) 1-5.
[26] V. E. Torres, et al., Tolvaptan in later-stage autosomal dominant polycystic kidney disease, N. Engl. J. Med. 377 (20) (2017) 1930-1942.
[27] F. T. Chebib, et al., A practical guide for treatment of rapidly progressive ADPKD with Tolvaptan, J. Am. Soc. Nephrol. 29 (10) (2018) 2458.
[28] E. J. Chung, et al., In vivo biodistribution and clearance of peptide amphiphile micelles, Nanomedicine 11 (2) (2015) 479-487.
[29] M. Schiff, et al., Chronic disease and self-injection: ethnographic investigations into the patient experience during treatment, Rheumatol. Therapy 4 (2) (2017) 445-463.
[30] B. Homayun, X. Lin, H.-J. Choi, Challenges and recent Progress in Oral drug delivery Systems for Biopharmaceuticals, Pharmaceutics 11 (3) (2019) 129.
[31] S. B. Tiwari, A. R. Rajabi-Siahboomi, Extended-release oral drug delivery technologies: monolithic matrix systems, in: K. K. Jain (Ed.), in Drug Delivery Systems, Humana Press, Totowa, N J, 2008, pp. 217-243.
[32] A. Berardi, F. Baldelli Bombelli, Oral delivery of nanoparticles—let's not forget about the protein corona, Expert Opinion Drug Deliv. 16 (6) (2019) 563-566.
[33] J. J. Wang, et al., Recent advances of chitosan nanoparticles as drug carriers, Int. J. Nanomedicine 6 (2011) 765-774.
[34] D. A. Johann, et al., Risk factors for complications in peripheral intravenous catheters in adults: secondary analysis of a randomized controlled trial, Revista latino-americana de enfermagem 24 (2016) e2833.
[35] J. McLenon, M. A. M. Rogers, The fear of needles: a systematic review and metaanalysis, J. Adv. Nurs. 75 (1) (2019) 30-42.
[36] C. M. McMurtry, et al., Interventions for individuals with high levels of needle fear: systematic review of randomized controlled trials and quasi-randomized controlled trials, Clin. J. Pain 31 (10 Suppl) (2015) S109-S123.
[37] A. Taddio, et al., Survey of the prevalence of immunization non-compliance due to needle fears in children and adults, Vaccine 30 (32) (2012) 4807-4812.
[38] C. M. McMurtry, et al., Exposure-based interventions for the management of individuals with high levels of needle fear across the lifespan: a clinical practice guideline and call for further research, Cogn. Behav. Ther. 45 (3) (2016) 217-235.
[39] S. Wright, et al., Fear of needles nature and prevalence in general practice, Aust. Fam. Physician 38 (2009) 172-176.
[40] J. H. Hamman, G. M. Enslin, A. F. Kotz'e, Oral delivery of peptide drugs, BioDrugs 19 (3) (2005) 165-177.
[41] J. Reinholz, K. Landfester, V. Mail″ander, The challenges of oral drug delivery via nanocarriers, Drug Deliv. 25 (1) (2018) 1694-1705.
[42] K. R. Groschwitz, S. P. Hogan, Intestinal barrier function: molecular regulation and disease pathogenesis, J. Allergy Clin. Immunol. 124 (1) (2009) 3-22.
[43] L. M. Ensign, R. Cone, J. Hanes, Oral drug delivery with polymeric nanoparticles: the gastrointestinal mucus barriers, Adv. Drug Deliv. Rev. 64 (6) (2012) 557-570.
[44] S. M. Pond, T. N. Tozer, First-pass elimination basic concepts and clinical consequences, Clin. Pharmacokinet. 9 (1) (1984) 1-25.
[45] R. S. Mayank Sharma, Dinesh Kumar Jain, Nanotechnology based approaches for enhancing oral bioavailability of poorly water soluble antihypertensive drugs, Scientifica (2016) 2016.
[46] M. A. Mohammed, et al., An overview of chitosan nanoparticles and its application in non-parenteral drug delivery, Pharmaceutics 9 (4) (2017) 53.
[47] Y. Kawashima, et al., Mucoadhesive D L-lactide/glycolide copolymer Nanospheres coated with chitosan to improve Oral delivery of Elcatonin, Pharm. Dev. Technol. 5 (1) (2000) 77-85.
[48] D. Elieh-Ali-Komi, M. R. Hamblin, Chitin and chitosan: production and application of versatile biomedical Nanomaterials, Int. J. Adv. Res. 4 (3) (2016) 411-427.
[49] M. D. Lenardon, C. A. Munro, N. A. R. Gow, Chitin synthesis and fungal pathogenesis, Curr. Opin. Microbiol. 13 (4) (2010) 416-423.
[50] A. Tolaimate, et al., Contribution to the preparation of chitins and chitosans with controlled physico-chemical properties, Polymer 44 (26) (2003) 7939-7952.
[51] I. Younes, M. Rinaudo, Chitin and chitosan preparation from marine sources. Structure, properties and applications, Marine Drugs 13 (3) (2015) 1133-1174.
[52] R. C. F. Cheung, et al., Chitosan: an update on potential biomedical and pharmaceutical applications, Marine Drugs 13 (8) (2015) 5156-5186.
[53] B. Bellich, et al., “The good, the bad and the ugly” of chitosans, Marine Drugs 14 (5) (2016) 99.
[54] A. S. Prata, C. R. Grosso, Production of microparticles with gelatin and chitosan, Carbohydr. Polym. 116 (2015) 292-299.
[55] H. B. Tan, et al., Fabrication and evaluation of porous keratin/chitosan (KCS) scaffolds for effectively accelerating wound healing, Biomed. Environ. Sci. 28 (3) (2015) 178-189.
[56] N. Hoshyar, et al., The effect of nanoparticle size on in vivo pharmacokinetics and cellular interaction, Nanomed. (London, England) 11 (6) (2016) 673-692.
[57] N. P. Birch, J. D. Schiffman, Characterization of self-assembled polyelectrolyte complex nanoparticles formed from chitosan and pectin, Langmuir 30 (12) (2014) 3441-3447.
[58] J. Barthelmes, et al., Thiomer nanoparticles: stabilization via covalent crosslinking, Drug Deliv. 18 (8) (2011) 613-619.
[59] K. Bowman, K. W. Leong, Chitosan nanoparticles for oral drug and gene delivery, Int. J. Nanomedicine 1 (2) (2006) 117-128.
[60] K. Chandra Hembram, et al., Advances in preparation and characterization of chitosan nanoparticles for therapeutics, Artif. Cells Nanomed. Biotechnol. 44 (1) (2016) 305-314.
[61] K. Nagpal, S. K. Singh, D. N. Mishra, Chitosan nanoparticles: a promising system in novel drug delivery, Chem. Pharm. Bull. 58 (11) (2010) 1423-1430.
[62] P. Calvo, et al., Novel hydrophilic chitosan-polyethylene oxide nanoparticles as protein carriers, J. Appl. Polym. Sci. 63 (1) (1997) 125-132.
[63] E. N. Koukaras, et al., Insight on the formation of chitosan nanoparticles through Ionotropic gelation with Tripolyphosphate, Mol. Pharm. 9 (10) (2012) 2856-2862.
[64] S. Sreekumar, et al., Parameters influencing the size of chitosan-TPP nano- and microparticles, Sci. Rep. 8 (1) (2018) 4695.
[65] V. Kamat, D. Bodas, K. Paknikar, Chitosan nanoparticles synthesis caught in action using microdroplet reactions, Sci. Rep. 6 (1) (2016) 22260.
[66] A. Pant, J. S. Negi, Novel controlled ionic gelation strategy for chitosan nanoparticles preparation using TPP—B-CD inclusion complex, Eur. J. Pharm. Sci. 112 (2018) 180-185.
[67] Z. He, et al., Scalable fabrication of size-controlled chitosan nanoparticles for oral delivery of insulin, Biomaterials 130 (2017) 28-41.
[68] M. Dur'an-Lobato, Z. Niu, M. J. Alonso, Oral delivery of biologics for precision medicine, Adv. Mater. 32 (13) (2020) 1901935.
[69] M. H. El-Shabouri, Positively charged nanoparticles for improving the oral bioavailability of cyclosporin-A, Int. J. Pharm. 249 (1) (2002) 101-108.
[70] A. P. Bagre, K. Jain, N. K. Jain, Alginate coated chitosan core shell nanoparticles for oral delivery of enoxaparin in vitro and in vivo assessment, Int. J. Pharm. 456 (1) (2013) 31-40.
[71] A. J. Scheen, Clinical pharmacokinetics of metformin, Clin. Pharmacokinet. 30 (5) (1996) 359-371.
[72] H. P. Chhetri, P. Thapa, A. Van Schepdael, Simple HPLC-UV method for the quantification of metformin in human plasma with one step protein precipitation, Saudi Pharmaceutical J.: SPJ: Off. Publ. Saudi Pharmaceutical Soc. 22 (5) (2014) 483-487.
[73] M. S. Arayne, et al., Spectrophotometric quantitation of metformin in bulk drug and pharmaceutical formulations using multivariate technique, Indian J. Pharm. Sci. 71 (3) (2009) 331-335.
[74] R. M. P. da Silva, J. F. Mano, R. L. Reis, Straightforward determination of the degree of N-acetylation of chitosan by means of first-derivative UV spectrophotometry, Macromol. Chem. Phys. 209 (14) (2008) 1463-1472.
[75] S. Klein, The use of biorelevant dissolution media to forecast the in vivo performance of a drug, AAPS J. 12 (3) (2010) 397-406.
[76] M. Natoli, et al., Good Caco-2 cell culture practices, Toxicol. in Vitro 26 (8) (2012) 1243-1246.
[77] M. Bens, et al., Corticosteroid-dependent sodium transport in a novel immortalized mouse collecting duct principal cell line, J. Am. Soc. Nephrol. 10 (5) (1999) 923.
[78] K. Watanabe, et al., Effect of insulin on cephalexin uptake and transepithelial transport in the human intestinal cell line Caco-2, Eur. J. Pharm. Sci. 21 (1) (2004) 87-95.
[79] J. Zhang, et al., Mechanism study of cellular uptake and tight junction opening mediated by goblet cell-specific Trimethyl chitosan nanoparticles, Mol. Pharm. 11 (5) (2014) 1520-1532.
[80] M. Ma, et al., Loss of cilia suppresses cyst growth in genetic models of autosomal dominant polycystic kidney disease, Nat. Genet. 45 (9) (2013) 1004-1012.
[81] J. A. Nieto, et al., An empirical biomarker-based calculator for cystic index in a model of autosomal recessive polycystic kidney disease—the Nieto-Narayan formula, PLOS One 11 (10) (2016) e0163063.
[82] N. Mohammadpourdounighi, et al., Preparation of chitosan nanoparticles containing Naja naja oxiana snake venom, Nanomedicine 6 (1) (2010) 137-143.
[83] A. Bozkir, O. M. Saka, Chitosan nanoparticles for plasmid DNA delivery: effect of chitosan molecular structure on formulation and release characteristics, Drug Deliv. 11 (2) (2004) 107-112.
[84] F. Brunel, et al., A novel synthesis of chitosan nanoparticles in reverse emulsion, Langmuir 24 (20) (2008) 11370-11377.
[85] K. G. Desai, Chitosan nanoparticles prepared by ionotropic gelation: an overview of recent advances, Crit. Rev. Ther. Drug Carrier Syst. 33 (2) (2016) 107-158.
[86] J. P. Qui nones, H. Peniche, C. Peniche, Chitosan based self-assembled nanoparticles in drug delivery, Polymers 10 (3) (2018) 235.
[87] M. Boegh, H. M. Nielsen, Mucus as a barrier to drug delivery—Understanding and mimicking the barrier properties, Basic Clin. Pharmacol. Toxicol. 116 (3) (2015) 179-186.
[88] J. Leal, H. D. C. Smyth, D. Ghosh, Physicochemical properties of mucus and their impact on transmucosal drug delivery, Int. J. Pharm. 532 (1) (2017) 555-572.
[89] T. M. M. Ways, W. M. Lau, V. V. Khutoryanskiy, Chitosan and its derivatives for application in mucoadhesive drug delivery systems, Polymers 10 (3) (2018) 267.
[90] A. L. Golub, et al., Physiologic considerations in drug absorption from the gastrointestinal tract, J. Allergy Clin. Immunol. 78 (4, Part 2) (1986) 689-694.
[91] J. M. Gamboa, K. W. Leong, In vitro and in vivo models for the study of oral delivery of nanoparticles, Adv. Drug Deliv. Rev. 65 (6) (2013) 800-810.
[92] M. Collado-Gonzalez, Y. Gonzalez Espinosa, F. M. Goycoolea, Interaction between chitosan and mucin: fundamentals and applications, Biomimetics (Basel, Switzerland) 4 (2) (2019) 32.
[93] M. M. Silva, et al., Chitosan nanoparticles as a mucoadhesive drug delivery system for ocular administration, Marine Drugs 15 (12) (2017) 370.
[94] H. M. Yildiz, et al., Size selectivity of intestinal mucus to diffusing particulates is dependent on surface chemistry and exposure to lipids, J. Drug Target. 23 (7-8) (2015) 768-774.
[95] S. P. Bandi, Y. S. Kumbhar, V. V. K. Venuganti, Effect of particle size and surface charge of nanoparticles in penetration through intestinal mucus barrier, J. Nanopart. Res. 22 (3) (2020) 62.
[96] M. J. Santander-Ortega, et al., PEGylated Nanoemulsions for Oral delivery: role of the inner Core on the final fate of the formulation, Langmuir 33 (17) (2017) 4269-4279.
[97] A. Muheem, et al., A review on the strategies for oral delivery of proteins and peptides and their clinical perspectives, Saudi Pharmaceutical J.: SPJ: Off. Publ. Saudi Pharmaceutical Soc. 24 (4) (2016) 413-428.
[98] J. Yang, et al., pH-responsive cellulose-chitosan nanocomposite films with slow release of chitosan, Cellulose 26 (6) (2019) 3763-3776.
[99] Z. H. Haliza Katas, Tay Chai Ling, Chitosan nanoparticles as a percutaneous drug delivery system for hydrocortisone, J. Nanomater. (2012) 2012.
[100] N. Islam, I. Dmour, M. O. Taha, Degradability of chitosan micro/nanoparticles for pulmonary drug delivery, Heliyon 5 (5) (2019), e01684.
[101] S. Suzuki, Experimental studies on the presumption of the time after food intake from stomach contents, Forensic Sci. Int. 35 (2) (1987) 83-117.
[102] A. M. Mohammed, et al., An overview of chitosan nanoparticles and its application in non-parenteral drug delivery, Pharmaceutics 9 (4) (2017).
[103] R. M. Saeed, I. Dmour, M. O. Taha, Stable chitosan-based nanoparticles using polyphosphoric acid or hexametaphosphate for tandem ionotropic/covalent crosslinking and subsequent investigation as novel vehicles for drug delivery, Front. Bioeng. Biotechnol. 8 (4) (2020).
[104] G. Sheetal, N. S. S. Roy, Ashish C. Mishra, Sandeep O. Waghulde, Mohan K. Kale, A review on chitosan nanoparticles applications in drug delivery, J. Pharmacog. Phytochem. 7 (6S) (2018) 01-04.
[105] K. Sonaje, et al., Opening of epithelial tight junctions and enhancement of Paracellular permeation by chitosan: microscopic, Ultrastructural, and computed tomographic observations, Mol. Pharm. 9 (5) (2012) 1271-1279.
[106] N. G. Lamson, et al., Anionic nanoparticles enable the oral delivery of proteins by enhancing intestinal permeability, Nat. Biomed. Eng. 4 (1) (2020) 84-96.
[107] G. Ranaldi, et al., The effect of chitosan and other polycations on tight junction permeability in the human intestinal Caco-2 cell line, J. Nutr. Biochem. 13 (3) (2002) 157-167.
[108] Y. Yun, Y. W. Cho, K. Park, Nanoparticles for oral delivery: targeted nanoparticles with peptidic ligands for oral protein delivery, Adv. Drug Deliv. Rev. 65 (6) (2013) 822-832.
[109] T. Dou, et al., Cellular uptake and transport characteristics of chitosan modified nanoparticles in Caco-2 cell monolayers, Int. J. Biol. Macromol. 138 (2019) 791-799.
[110] A. S. Macedo, et al., Novel and revisited approaches in nanoparticle systems for buccal drug delivery, J. Control. Release 320 (2020) 125-141.
[111] X. Liu, et al., Experimental evaluation of the transport mechanisms of PoIFN-α in Caco-2 cells, Front. Pharmacol. 8 (2017) 781.
[112] S. L. Seliger, et al., A randomized clinical trial of metformin to treat autosomal dominant polycystic kidney disease, Am. J. Nephrol. 47 (5) (2018) 352-360.
[113] A. Pisani, et al., Metformin in autosomal dominant polycystic kidney disease: experimental hypothesis or clinical fact? BMC Nephrol. 19 (1) (2018) 282.
[114] S. Meng, et al., Metformin activates AMP-activated protein kinase by promoting formation of the aBy heterotrimeric complex, J. Biol. Chem. 290 (6) (2015) 3793-3802.
[115] R. A. Miller, et al., Biguanides suppress hepatic glucagon signaling by decreasing production of cyclic AMP, Nature 494 (7436) (2013) 256-260.
[116] K. R. Hallows, et al., Inhibition of cystic fibrosis transmembrane conductance regulator by novel interaction with the metabolic sensor AMP-activated protein kinase, J. Clin. Invest. 105 (12) (2000) 1711-1721.
[117] C. J. Davidow, et al., The cystic fibrosis transmembrane conductance regulator mediates transepithelial fluid secretion by human autosomal dominant polycystic kidney disease epithelium in vitro, Kidney Int. 50 (1) (1996) 208-218.
[118] J. J. Reineke, et al., Unique insights into the intestinal absorption, transit, and subsequent biodistribution of polymer-derived microspheres, Proc. Natl. Acad. Sci. U.S.A 110 (34) (2013) 13803-13808.
[119] A. A. Date, J. Hanes, L. M. Ensign, Nanoparticles for oral delivery: design, evaluation and state-of-the-art, J. Control. Release: Off. J. Control. Release Soc. 240 (2016) 504-526.
[120] M.-H. Zou, et al., Activation of the AMP-activated protein kinase by the antidiabetic drug metformin in vivo: role of mitochondrial reactive nitrogen species, J. Biol. Chem. 279 (42) (2004) 43940-43951.
[121] L. A. Smith, et al., Development of polycystic kidney disease in juvenile cystic kidney mice: insights into pathogenesis, ciliary abnormalities, and common features with human disease, J. Am. Soc. Nephrol. 17 (10) (2006) 2821.
[122] Janzer M, Larbig G, Kübelbeck A, Wischnjow A, Haberkorn U, Mier W. Drug conjugation affects pharmacokinetics and specificity of kidney targeted peptide carriers. Bioconjug Chem. 2016; 27(10):2441-2449.
[123] 24. Du B J, Yu M X, Zheng J. Transport and interactions of nanoparticles in the kidneys. Nat Rev Mater. 2018; 3(10):358-374.quant
[124] 34. Kastantin M, Ananthanarayanan B, Karmali P, Ruoslahti E, Tirrell M. Effect of the lipid chain melting transition on the stability of DSPEPEG(2000) micelles. Langmuir. 2009; 25(13): 7279-7286.
[125] 35. Chung E J, Mlinar L B, Nord K, et al. Monocyte-targeting supramolecular micellar assemblies: a molecular diagnostic tool for atherosclerosis. Adv Healthc Mater. 2015; 4(3):367-376.
[126] Zuckerman J E, Choi C H J, Han H, Davis M E. Polycation-siRNA nanoparticles can disassemble at the kidney glomerular basement membrane. Proc Natl Acad Sci USA. 2012; 109(8):3137-3142.
[127] Pombo Garcia K, Zarschler K, Barbaro L, et al. Zwitterionic-coated “stealth” nanoparticles for biomedical applications: recent advances in countering biomolecular corona formation and uptake by the mononuclear phagocyte system. Small. 2014; 10(13):2516-2529.
[128] Murthy A K, Stover R J, Hardin W G, et al. Charged gold nanoparticles with essentially zero serum protein adsorption in undiluted fetal bovine serum. J Am Chem Soc. 2013; 135(21): 7799-7802.
[129] Liu W, Choi H S, Zimmer J P, Tanaka E, Frangioni J V, Bawendi M. Compact cysteine-coated CdSe(ZnCdS) quantum dots for in vivo applications. J Am Chem Soc. 2007; 129(47): 14530-14531.
[130] Kane R S, Deschatelets P, Whitesides G M. Kosmotropes form the basis of protein-resistant surfaces. Langmuir. 2003; 19(6):2388-2391.
[131] Piatkovsky M, Acar H, Marciel A B, Tirrell M, Herzberg M. A zwitterionic block-copolymer, based on glutamic acid and lysine, reduces the biofouling of UF and RO membranes. J Membr Sci. 2018; 549:507-514.
[132] Owens D E 3rd, Peppas N A. Opsonization, biodistribution, and pharmacokinetics of polymeric nanoparticles. Int J Pharm. 2006; 307(1): 93-102.
[133] Estephan Z G, Jaber J A, Schlenoff J B. Zwitterion-stabilized silica nanoparticles: toward nonstick nano. Langmuir. 2010; 26(22): 16884-16889.
[134] Holmlin R E, Chen X, Chapman R G, Takayama S, Whitesides G M. Zwitterionic S AMs that resist nonspecific adsorption of protein from aqueous buffer. Langmuir. 2001; 17(9):2841-2850.
[135] Liang X, Wang H, Zhu Y, et al. Short- and long-term tracking of anionic ultrasmall nanoparticles in kidney. ACS Nano. 2016; 10(1): 387-395.
[136] Balogh L, Nigavekar S S, Nair B M, et al. Significant effect of size on the in vivo biodistribution of gold composite nanodevices in mouse tumor models. Nanomedicine. 2007; 3(4):281-296.
[137] Comper W D, Glasgow E F. Charge selectivity in kidney ultrafiltration. Kidney Int. 1995; 47(5): 1242-1251.
[138] Perry M A, Benoit J N, Kvietys P R, Granger D N. Restricted transport. 1983; 245(4): G568-G572.
[139] Xiao K, Li Y, Luo J, et al. The effect of surface charge on in vivo biodistribution of PEG-oligocholic acid based micellar nanoparticles. Biomaterials. 2011; 32(13):3435-3446.
[140] LaConte L E, Nitin N, Zurkiya O, et al. Coating thickness of magnetic iron oxide nanoparticles affects R2 relaxivity. J Magn Reson Imaging. 2007; 26(6): 1634-1641.
[141] Tauris J, Christensen E I, Nykjær A, Jacobsen C, Petersen C M, Ovesen T. Cubilin and megalin co-localize in the neonatal inner ear. Audiol Neurootol. 2009; 14(4):267-278.
[142] Christensen E I, Birn H. Megalin and cubilin: multifunctional endocytic receptors. Nat Rev Mol Cell Biol. 2002; 3(4):256-266.
[143] Christensen E I, Birn H, Verroust P, Moestrup S K. Megalin-mediated endocytosis in renal proximal tubule. Ren Fail. 1998; 20(2): 191-199.
[144] Vegt E, Melis M, Eek A, et al. Renal uptake of different radiolabeled peptides is mediated by megalin: SPECT and biodistribution studies in megalin-deficient mice. Eur J Nucl Med Mol Imaging. 2011; 38(4): 623-632.
[145] Poon C, Chowdhuri S, Kuo C-H, et al. Protein mimetic and anticancer properties of monocyte-targeting peptide Amphiphile micelles. ACS Biomater Sci Eng. 2017; 3(12):3273-3282.
[146] Poon C, Gallo J, Joo J, Chang T, Bañobre-López M, Chung E J. Hybrid, metal oxide-peptide amphiphile micelles for molecular magnetic resonance imaging of atherosclerosis. J Nanobiotechnol. 2018; 16:1-11.
[147] Rodrigues W F, Miguel C B, Napimoga M H, Oliveira C J F, Lazo-Chica J E. Establishing standards for studying renal function in mice through measurements of body size-adjusted creatinine and urea levels. Biomed Res Int. 2014; 2014:872827.
[148] Dunn S R, Qi Z, Bottinger E P, Breyer M D, Sharma K. Utility of endogenous creatinine clearance as a measure of renal function in mice. Kidney Int. 2004; 65(5): 1959-1967.
[149] 62. Li M, Corbelli A, Watanabe S, et al. Three-dimensional podocyte endothelial cell co-cultures: assembly, validation, and application to drug testing and intercellular signaling studies. Eur J Pharm Sci. 2016; 86:1-12.

1. A drug delivery system for oral administration comprising a plurality of nanoparticles, each drug delivery system including:
an oral delivery carrier; and
a plurality of micelles attached to or dispersed in the oral delivery carrier, each micelle including a hydrophobic core and a hydrophilic corona targeted to a subject's kidney; and
a pharmaceutical payload carried by the plurality of micelles.

2. The drug delivery system of claim 1 wherein the hydrophilic corona includes a kidney targeting peptide conjugated to the hydrophobic core with a linking compound.

3. The drug delivery system of claim 2 wherein the kidney targeting peptide includes a sequence selected from the group consisting of KKEEE (SEQ ID NO: 1), KKEEEK (SEQ ID NO: 2), KKEEEKKEEE (SEQ ID NO: 3), KKEEEKKEEEK (SEQ ID NO: 4), KKEEEKKEEEKKEEE (SEQ ID NO: 5), and KKEEEKKEEEKKEEEK (SEQ ID NO: 6).

4. The drug delivery system of claim 2 wherein the kidney targeting peptide includes a sequence selected from the group consisting of EEKKK (SEQ ID NO: 7), EEKKKE (SEQ ID NO: 8), EEKKKEEKKK (SEQ ID NO: 9), EEKKKEEKKKE (SEQ ID NO: 10), and EEKKKEEKKKEEE (SEQ ID NO: 11), and EEKKKEEKKKEEEK (SEQ ID NO: 12).

5. The drug delivery system of claim 2 wherein the kidney targeting peptide includes a sequence selected from the group consisting of EEEEE (SEQ ID NO: 13), KKKKK (SEQ ID NO: 14), MGSHIEPGG (SEQ ID NO: 15), KMGGTNHPE (SEQ ID NO: 16), GRGDSP (SEQ ID NO: 17), ELRGDRAKL (SEQ ID NO: 18), and CKDSPKSSKSIRFIPVST (SEQ ID NO: 19).

6. The drug delivery system of claim 2 including one or more targeting peptides.

7. The drug delivery system of claim 2 wherein the kidney targeting peptide is selected from the group consisting of SEQ ID Nos: 1-19 with a cysteine added to the N-terminus or C-terminus thereof.

8. The drug delivery system of claim 2 wherein the kidney targeting peptide is connected to the micelles by reaction with a functional group attached to an end of a polyethylene glycol linking group.

9. The drug delivery system of claim 8 wherein the functional group that can be used for linking includes amines, carboxylic acids, NHS esters, acid anhydrides, or unsaturated imides (e.g., maleimide).

10. The drug delivery system of claim 2 wherein the linking compound is polyethylene glycol having a weight average molecular weight from about 500 Daltons to 10000 Daltons.

11. The drug delivery system of claim 2 wherein the linking compound is polyethylene glycol having a weight average molecular weight less than or equal to 1800 Daltons.

12. The drug delivery system of claim 1 wherein the pharmaceutical payload is located in a micelle surface and/or a micelle middle and/or a micelle core.

13. The drug delivery system of claim 1 wherein the pharmaceutical payload includes one or more pharmaceutical compounds.

14. The drug delivery system of claim 1 wherein the pharmaceutical payload is an mTOR inhibitor.

15. The drug delivery system of claim 1 wherein the pharmaceutical payload is selected from the group consisting of pioglitazone, niacinamide, rapamycin, everolimus, tesevatinib, tolvaptan, metformin, somatostatin, octreotide, pasireotide, lixivaptan, venglustat, bardoxolone methyl, salsalate, curcumin, and combinations thereof.

16. The drug delivery system of claim 1 wherein the pharmaceutical payload is selected from the group consisting of epigenetic modifying drugs including DNA methyltransferase inhibitors (especially Acytidine, Decitabine, RG108) and histone deacetylase inhibitors (especially Trichostatin A).

17. The drug delivery system of claim 1 wherein the pharmaceutical payload is a pravastatin, another statin, or combinations thereof.

18. The drug delivery system of claim 1 wherein the pharmaceutical payload includes nucleic acids such as microRNA, messenger RNA (mRNA), aptamers, antibodies, and/or lectins.

19. The drug delivery system of claim 1 wherein the pharmaceutical payload includes a microRNA-17 inhibitor.

20. The drug delivery system of claim 1 wherein the pharmaceutical payload includes mRNA encoding PKD1, PKD2, and/or PKHD1.

21. The drug delivery system of claim 1 wherein the oral delivery carrier includes an enteric coating and/or gelatin.

22. The drug delivery system of claim 21 wherein the oral delivery carrier is an enteric coating that includes a component selected from the group consisting of cellulose acetate, hydroxypropyl methyl cellulose, methyl acrylate, and combinations thereof.

23. The drug delivery system of claim 1 wherein the oral delivery carrier includes chitosan.

24. The drug delivery system of claim 23 wherein the chitosan includes crosslinked chitosan.

25. The drug delivery system of claim 24 wherein crosslinked chitosan includes chitosan crosslinked with poly-glutamic acid or tripolyphosphate.

26. The drug delivery system of claim 24 wherein crosslinked chitosan includes acetylated chitosan having a degree of acetylation from about 70 to 98 mole percent.

27. The drug delivery system of claim 1 wherein each micelle includes a plurality of targeting peptide-conjugated amphiphiles and a plurality of non-targeted amphiphiles, the plurality of targeting peptide-conjugated amphiphiles including amphiphiles having a targeting peptide selected from the group consisting of SEQ ID NOs 1-19 conjugated to a base amphiphile and SEQ ID NOs 1-19 with a cysteine added to the N-terminus or C-terminus thereof.

28. The drug delivery system of claim 27 wherein the plurality of targeting peptide-conjugated amphiphiles include amphiphiles having a phospholipid conjugated to the targeting peptide with a linking group.

29. The drug delivery system of claim 28 wherein the phospholipid is selected from the group consisting of phosphatidic acids, phosphatidyl inositols, phosphatidyl cholines, phosphatidyl ethanolamines, phosphatidyl serines, phosphatidyl glycerols, and any combinations thereof.

30. The drug delivery system of claim 28 wherein the phospholipid is selected from the group consisting of phosphatidylglycerol, lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine, palmitoyloleoylphosphatidylcholine, di stearoylphosphatidylcholine, dioleoylphosphatidylcholine, dipalmitoylphosphatidylcholine, dioleoylphosphatidylglycerol, dipalmitoylphosphatidylglycerol, -phosphatidylethanolamine, dioleoyl-phosphatidylethanolamine 4-(N-maleimidomethyl)-cyclohexane-1-carboxylate (DOPE-mal), 1-stearoyl-2-oleoyl phosphatidylcholine, 1,2-distearoyl-sn-glycerol-3-phosphoethanolamine, and combinations thereof.

31. The drug delivery system of claim 27 wherein the plurality of non-targeted amphiphiles includes amphiphiles having formula:

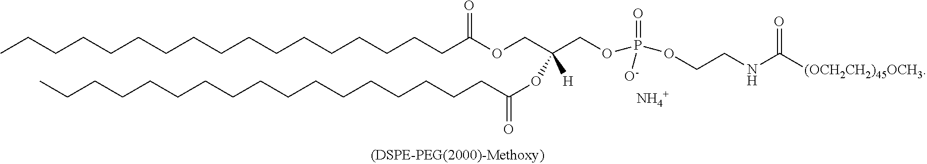

32. A method for treating kidney disease in a subject, the method including a step of administering a therapeutically effective amount of the drug delivery system of claim 1 to the subject.

33. The method of claim 32 wherein the kidney disease is a chronic or acute kidney disease.

34. The method of claim 32 wherein the kidney disease is diabetic kidney disease, a tubulointerstitial disease, glomerulonephritis, Alport Syndrome, or polycystic kidney disease.

35. The method of claim 32 wherein the kidney disease is polycystic kidney disease.

36. A drug delivery system comprising:
a plurality of micelles, each micelle having a kidney targeting peptide conjugated thereto with a polyethylene glycol linking group having a molecular weight less than 1800 Daltons, and
a payload conjugated to or encapsulated by each micelle.

37. The drug delivery system of claim 36 wherein the kidney targeting peptide includes a sequence selected from the group consisting of KKEEE (SEQ ID NO: 1), KKEEEK (SEQ ID NO: 2), KKEEEKKEEE (SEQ ID NO: 3), KKEEEKKEEEK (SEQ ID NO: 4), KKEEEKKEEEKKEEE (SEQ ID NO: 5), and KKEEEKKEEEKKEEEK (SEQ ID NO: 6).

38. The drug delivery system of claim 36 wherein the kidney targeting peptide includes a sequence selected from the group consisting of EEKKK (SEQ ID NO: 7), EEKKKE (SEQ ID NO: 8), EEKKKEEKKK (SEQ ID NO: 9), EEKKKEEKKKE (SEQ ID NO: 10), and EEKKKEEKKKEEE (SEQ ID NO: 11), and EEKKKEEKKKEEEK (SEQ ID NO: 12).

39. The drug delivery system of claim 36 wherein the kidney targeting peptide includes a sequence selected from the group consisting of EEEEE (SEQ ID NO: 11), KKKKK (SEQ ID NO: 12), MGSHIEPGG (SEQ ID NO: 15), KMGGTNHPE (SEQ ID NO: 16), GRGDSP (SEQ ID NO: 17), ELRGDRAKL (SEQ ID NO: 18), and CKDSPKSSKSIRFIPVST (SEQ ID NO: 19).

40. The drug delivery system of claim 36 wherein the kidney targeting peptide is selected from the group consisting of SEQ ID Nos: 1-19 with a cysteine added to the N-terminus or C-terminus thereof.

41-59. (canceled)
